(19) United States
(12) Patent Application Publication
(10) Pub. No.: US 2024/0052374 A1
(43) Pub. Date: Feb. 15, 2024

(54) PRODUCTION OF DITERPENE ALKALOIDS

(71) Applicants: Garret P. Miller, Waltham, MA (US); Björn Hamberger, Okemos, MI (US); Imani Pascoe, East Lansing, MI (US); Kathryn Van Winkle, Medford, MA (US)

(72) Inventors: Garret P. Miller, Waltham, MA (US); Björn Hamberger, Okemos, MI (US); Imani Pascoe, East Lansing, MI (US); Kathryn Van Winkle, Medford, MA (US)

(21) Appl. No.: 18/357,767

(22) Filed: Jul. 24, 2023

Related U.S. Application Data
(60) Provisional application No. 63/369,148, filed on Jul. 22, 2022.

(51) Int. Cl. C12P 5/00; C12N 5/04

(52) U.S. Cl. CPC C12P 5/007; C12N 5/04

(57) ABSTRACT

Enzymes and methods are described herein for manufacturing terpenes, including diterpenoid alkaloids.

### CROSS-REFERENCE TO RELATED APPLICATION

This application claims the priority of U.S. provisional application Ser. No. 63/369,148, filed Jul. 22, 2022, the disclosure of which is incorporated herein by reference in its entirety.

### FEDERAL FUNDING

This invention was made with government support under 1737898 awarded by the National Science Foundation. The government has certain rights in the invention.

### INCORPORATION BY REFERENCE OF SEQUENCE LISTING

A Sequence Listing is provided herewith as an xml file, “2353460.xml” created on Jul. 24, 2023, and having a size of 30,896 bytes. The content of the xml file is incorporated by reference herein in its entirety.

### BACKGROUND

The roots from the Aconitum (Wolf's Bane) and Delphinium (Larkspur) genera have been used in traditional medicine owing to the abundance of bioactive diterpenoid alkaloids that they produce. Many compounds are produced by both genera. However, despite a wealth of studies on different medicinal properties of these metabolites as well as efforts towards total chemical synthesis, very little progress has been made towards elucidation of the biosynthetic pathways for these compounds.

### SUMMARY

Described herein are several of the entry steps in the biosynthesis of diterpenoid alkaloids. Seven enzymes have been identified from Siberian Larkspur (Delphinium grandiflorum). The biosynthetic pathway can include one or more of two terpene synthases described herein, one or more of the four cytochrome P450s described herein, and/or a reductase described herein that has little homology to other characterized enzymes. Three of the newly described cytochrome P450s are the founding members of new subfamilies with one belonging to the poorly characterized CYP729 family. These enzymes and production of a key intermediate in a heterologous host provides biosynthetic production of a group of metabolites such as diterpenoid alkaloids that are useful for medicinal applications.

Described herein are methods and expression systems that can provide diterpenoid alkaloids. For example, expression systems are described herein that include at least one expression cassette having a heterologous promoter operably linked to a nucleic acid segment encoding an enzyme with at least 90% sequence identity to SEQ ID NO:1, 3, 5, 7, 9, 11, or 13. Also described herein are host cells that include such expression systems.

Methods of synthesizing a diterpenoid alkaloids are also described herein. For example, such methods of synthesizing a diterpenoid alkaloid can include incubating a host cell that has such expression system. The host cell can be supplied a precursor for synthesis of the diterpenoid alkaloid such as geranylgeranyl diphosphate (GGPP). In some cases, one or more of the enzyme(s) with at least 90% sequence identity to SEQ ID NO:1, 3, 5, 7, 9, 11, or 13 are incubated inn vitro with at least one precursor for a diterpenoid alkaloid, such as geranylgeranyl diphosphate (GGPP).

### DESCRIPTION OF THE FIGURES

FIG. 1 is a maximum likelihood phylogenetic tree of predicted D. grandiflorum terpene synthase (TPS) sequences. Only eight out of the fifteen predicted sequences are shown, as many resulted in only partial transcripts with low coverage against reference sequences. Labels at branch points indicate percent bootstrap support from 1,000 replicates. Names with an arrow had root-exclusive expression, and DgrTPS1 and DgrTPS7 were functionally characterized.

FIGS. 2A-2C are graphs depicting retention time and mass spectra of a product of DgrTPS1, an ent-CPP synthase. FIG. 2A is a graph of the retention time showing that transient expression of DgrTPS1 in N. benthamiana yields a product with the same retention time and mass spectra as ZmAN2 (ent-CPP synthase) and NmTPS1 ((+)-CPP synthase; (+)-CPP is the enantiomer of the structure drawn for the grayscale region). The absolute stereochemistry of DgrTPS1's product was determined through coexpression of an enantioselective ent-kaurene synthase (NmTPS2), which converts only the ent enantiomer of CPP to ent-kaurene. Each assay has CfDXS and CfGGPPS coexpressed in addition to those listed. FIG. 2B is a mass spectrum of dephosphorylated ent-CPP. FIG. 2C is a mass spectrum of ent-kaurene.

FIGS. 3A-3B are graphs depicting retention time and mass spectra of a product of DgrTPS7a and DgrTPS7b that convert ent-CPP to ent-atiserene. FIG. 3A is a graph of the retention time showing that transient expression of DgrTPS7a and DgrTPS7b yield ent-atiserene when coexpressed with an ent-CPP synthase (ZmAN2 or DgrTPS1). DgrTPS7a and DgrTPS7b are also enantioselective and do not convert (+)-CPP (from NmTPS1) to a new product. Each assay has CfDXS and CfGGPPS coexpressed in addition to those listed. FIG. 3B is a mass spectrum of ent-atiserene.

FIGS. 4A-4D are graphs depicting retention time and mass spectra of products of CYP701A127 and CYP71FH1 that convert ent-atiserene to oxidized products. FIG. 4A is a graph of retention time showing that coexpression of either CYP701A127 and CYP71FH1 with DgrTPS1 and DgrTPS7 result in depletion of ent-atiserene and production of oxidized products, while the remainder of candidates show a similar accumulation of ent-atiserene to DgrTPS1 and DgrTPS7 alone. CYP701A127 likely makes ent-atiserene-19-al (as such is drawn in gray in the second graph from the top). CYP71FH1 makes ent-atiserene-20-al (confirmed by NMR) and another major product (C) with an unsolved structure. Each assay has CfDXS, CfGGPPS, DgrTPS1, and DgrTPS7 coexpressed in addition to those listed. FIG. 4B is a mass spectrum of aldehyde product ent-atiserene-19-al. FIG. 4C is a mass spectrum of aldehyde product ent-atiserene-20-al. FIG. 4D is a mass spectrum of unknown C. Mass spectra for minor products A and B have a molecular ion of 302 m/z and are given in FIGS. 8A-D.

FIGS. 5A-5B are graphs depicting retention time and mass spectra showing that coexpression of CYP701A127 and CYP71FH1 lead to an accumulation of the same products. FIG. 5A is a graph of retention time showing GC-MS (top panel) and LC-MS (bottom panel) analysis of CYP701A127 and CYP71FH1 coexpression. Individual products of either enzyme detectable by GC-MS are no longer present when both are coexpressed. Products detectable by LC-MS for CYP701A127 are depleted upon coexpression of both P450s, however those for CYP71FH1 accumulate. One additional peak is seen upon coexpression of both enzymes (compound J). Each assay has CfDXS, CfGGPPS, DgrTPS1, and DgrTPS7 coexpressed in addition to those listed. FIG. 5B shows mass spectra and predicted chemical formulas for three products: compound G (top panel), compound H (middle panel), and compound I (bottom panel). Mass spectra for products not shown here are given in FIGS. 10A-B.

FIGS. 6A-6B are graphs depicting retention time and mass spectra showing that CYP729G1 and CYP71FK1 have redundant functions. FIG. 6A is a graph of retention time showing GC-MS (top six spectra) and LC-MS (bottom two spectra) analysis of CYP701A127 and CYP71FH1 coexpression. Individual products of either enzyme detectable by GC-MS are no longer present when both are coexpressed. Products detectable by LC-MS for CYP701A127 are depleted upon coexpression, however those for CYP71FH1 accumulate. One additional peak is seen upon coexpression of both enzymes (compound J). Each assay has CfDXS, CfGGPPS, DgrTPS1, and DgrTPS7 coexpressed in addition to those listed. FIG. 6B shows mass spectra and predicted chemical formulas for three products: compound M (top panel), compound 0 (middle panel), and compound N (bottom panel). Mass spectra for products not shown here are given in FIGS. 11A-B.

FIGS. 7A-7B are graphs depicting retention time and mass spectra showing coexpression with SangRed produces an isomer of what is produced upon supplementation with ethylamine. FIG. 7A is an LC-MS analysis of SangRed and AlaDC coexpression with previous steps of the pathway. Products G, H, and I from the first four enzymes are depleted upon coexpression with SangRed, and a new product P is made. Compound P has an identical exact mass to a minor product R, which is made through coexpression of AlaDC. A new compound Q is made through coexpression of SangRed with the first four enzymes and CYP729G1. Compound Q similarly has an identical exact mass to a minor product S, which is made through coexpression of AlaDC. Each assay has CfDXS, CfGGPPS, DgrTPS1, and DgrTPS7 coexpressed in addition to those listed. FIG. 7B shows mass spectra and predicted chemical formulas for compounds made through coexpression with either SangRed or AlaDC and a putative difference of one hydroxylation upon addition of CYP729G1.

FIGS. 8A-8D are mass spectra for the compounds shown in FIG. 4A. FIG. 8A is a section of FIG. 4A showing the second and third spectra. FIG. 8B show mass spectra for compounds made through coexpression of CYP701A127 with previous pathway steps. FIG. 8C show close matches for ent-atiserene-19-al from the NIST database with mass spectra and structures. FIG. 8D show mass spectra for compounds made through coexpression of CYP71FH1 with previous pathway steps.

FIG. 9 is a structure illustrating HMBC correlations for ent-atiserene-20-al which show methyl groups for carbons 18 and 19 are retained following conversion of ent-atiserene by CYP71FH1.

FIGS. 10A-B show mass spectra for compounds shown in FIG. 5A. FIG. 10A shows the bottom three mass spectra shown in FIG. 5A. FIG. 10B shows the mass spectra of the compounds D, E, F, G, H, I, and J shown in FIG. 10A.

FIGS. 11A-B show mass spectra for compounds shown in FIG. 6A. FIG. 11A shows the bottom two mass spectra shown in FIG. 6A. FIG. 11B shows the mass spectra of the compounds K, L, M, N, and O shown in FIG. 11A.

FIG. 12 is a graph depicting retention time showing that CYP729G1 and CYP71FK1 have similar activity when coexpressed with SangRed. Each assay has CfDXS, CfGGPPS, DgrTPS1, and DgrTPS7 coexpressed in addition to those listed.

### DETAILED DESCRIPTION

Alkaloids are a diverse class of compounds broadly defined as nitrogen-containing specialized metabolites. Diterpenoid alkaloids are natural compounds having complex structural features with many stereo-centers originating from the amination of natural tetracyclic diterpenes and produced primarily from plants in the Aconitum, Delphinium, and/or Consolida genera. Diterpene alkaloids are derived from tetracyclic or pentacyclic diterpenes in which carbon atoms 19 and 20 are linked with the nitrogen of a molecule of β-aminoethanol, methylamine, or ethylamine to form a heterocyclic ring. These alkaloids may be divided into two broad categories. The first group comprises the highly toxic ester bases that are heavily substituted by methoxyl and hydroxyl groups. The second group includes a series of comparatively simple and relatively nontoxic alkamines that are modeled on a C₂₀-skeleton. One of the distinguishing chemical features of this group is the formation of phenanthrenes when subjected to selenium or palladium dehydrogenation. A few compounds of this class occur in the plant as monoesters of acetic or benzoic acid.

Many examples of plant alkaloids have received attention for their medicinal applications. Prominent examples include alkaloids such as morphine¹ (analgesic), colchicine² (anti-inflammatory), scopolamine³⁻⁵ (anti-nausea), and vinblastine⁶⁻⁸ (anti-cancer). Much like terpenoids, the entry steps to the biosynthesis of many of these compounds involve an initial scaffold formation and is followed by modifications by enzymes such as P450 enzymes and methyltransferases and acetyltransferases.

Rather than a carbocation-mediated cyclization of a single molecule as in terpenoid biosynthesis, the scaffold-forming step in alkaloid biosynthesis typically involves the accumulation and condensation of an amine and aldehyde precursor, followed by resolution of the resulting iminium cation to form an alkaloid scaffold⁹. Given the unique pathways towards initial scaffold formation, there is little overlap between the terpenoid and alkaloid classes of specialized metabolites.

One notable exception is the monoterpenoid indole alkaloids, derived from tryptophan and geranyl diphosphate (GPP). Decarboxylation of tryptophan into tryptamine leads to the accumulation of a primary amine, and conversion of GPP to secologanin leads to the accumulation of an aldehyde, which condense to form the initial scaffold towards monoterpenoid indole alkaloid metabolites⁸. Another exception are the diterpenoid alkaloids, which are found in at least 4 independent plant lineages¹⁰⁻¹²—most notably within the Ranunculaceae family^{13,14}. The biosynthesis of this class of metabolites has not been elucidated, however it is apparent from their structure that it involves the initial formation of a diterpene scaffold and nitrogen incorporation follows, in contrast to the monoterpenoid indole alkaloids where the terpene precursor is not first cyclized by a terpene synthase and does not make up the majority of the scaffold⁸.

Plants from the Aconitum and Delphinium genera have been used in traditional medicine due to of the bioactivity of these diterpenoid alkaloids. “Fuzi,” the processed lateral root of A. carmichaelii (more commonly known as Wolf's Bane or Aconite), has been used for at least two thousand years¹⁴. The diterpenoid alkaloids have a wide range of applications from antifeedants to anti-cancer, choline esterase inhibitors, and analgesics¹³⁻¹⁶. The therapeutic properties of many of these metabolites has prompted research into total chemical synthesis of specific compounds¹⁷⁻²¹, however the structural complexity of these compounds presents an enormous challenge in chemical synthesis. Aconitine (one such compound which is a potent neurotoxin), for example, contains six interconnected rings and fifteen stereocenters.

Elucidating the biosynthesis of these compounds could ameliorate the challenges involved their production. Such challenges relate to the complexity of their scaffolds and number of required stereospecific oxidations. The lack of current knowledge in their biosynthesis is not for a lack of effort, as many previous attempts have been made to elucidate biosynthetic genes through transcriptomic analysis in various Aconitum species²²⁻²⁶, with only one case published recently which characterized a pair of terpene synthases (TPSs)²⁷.

The following schematic (Scheme 1) illustrates common structural features of diterpenoid alkaloids and the biosynthetic pathway elucidated as described herein. Bonds shaded in gray highlight a common labdane structure likely derived from activity of a class II TPS (shown as a dotted line in aconitine due to a ring expansion proposed to happen further in the pathway). Carbons within shaded circles have common stereochemistry. Bonds with arrows show the same three-carbon bridges that make up either side of a six-membered ring. Carbons within unfilled circles represent methyl groups on ent-atiserene which are likely converted to aldehydes to allow for nitrogen incorporation.

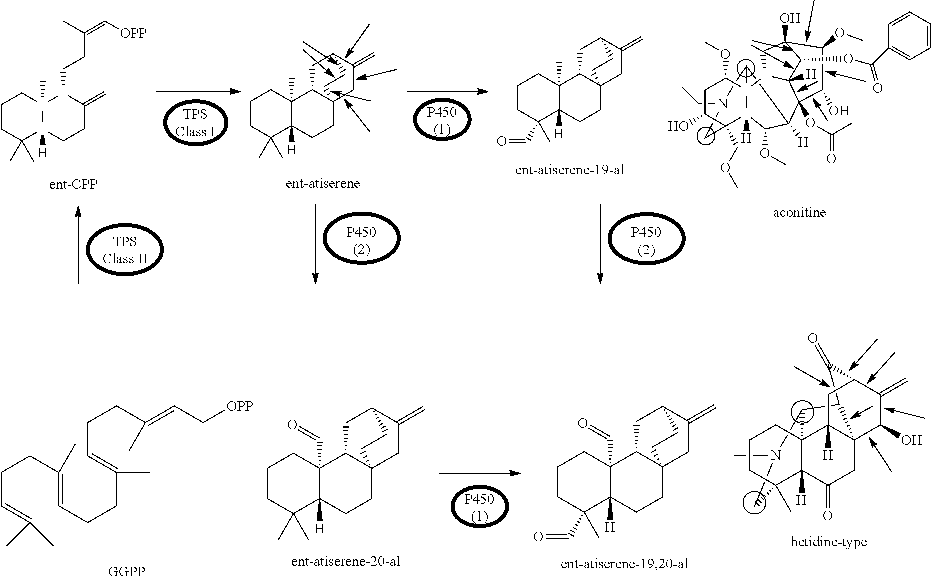

A variety of diterpenoid alkaloids can be made using the expression systems, enzymes, and methods described herein. As illustrated herein, the first committed key steps have been identified, and starting scaffold for the majority of diterpenoid alkaloids in the Ranunculaceae family. These are characterized by a labdanoid starting diterpene and have a 6/6/6/6 or 6/7/5/6 ring structure, as shown in the schematic above. Characteristic diterpenoid alkaloids include aconitine and hetidine-type and it is suggested herein that they are derived from the same starting point, ent-atiserene. Key functionalization steps are described herein that are catalyzed by novel enzymes of the cytochrome P450 class and the incorporation of the nitrogen is shown, yielding the alkaloid structure.

Examples of diterpenoid alkaloids include the following.

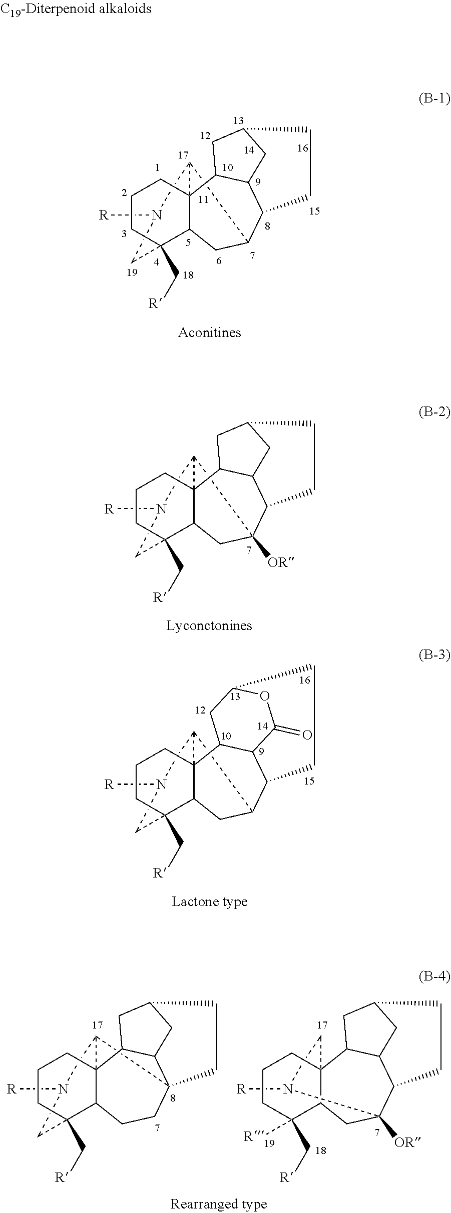

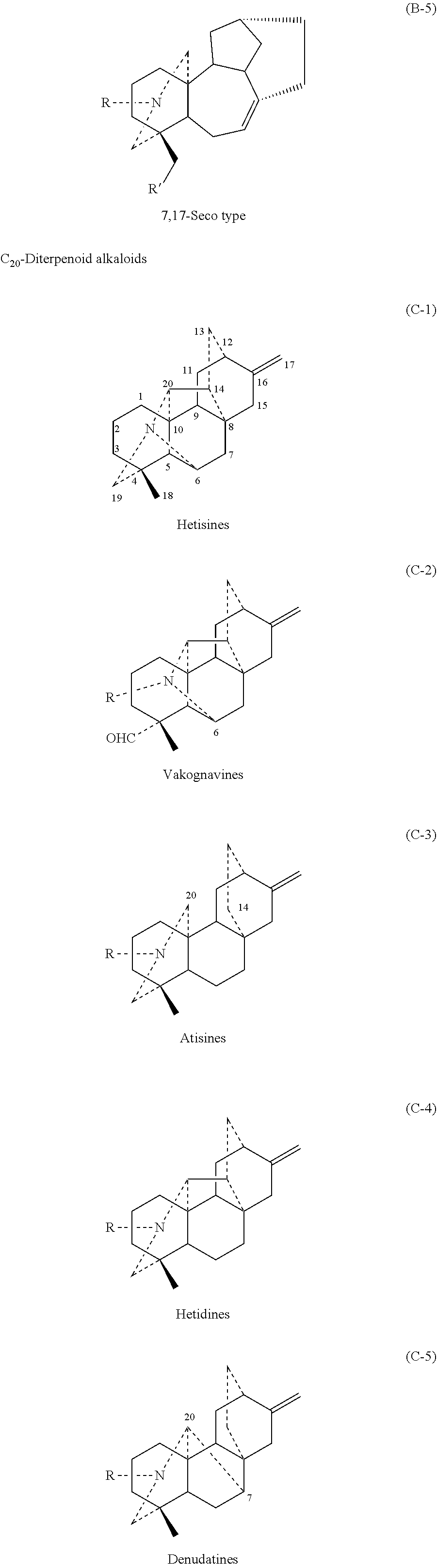

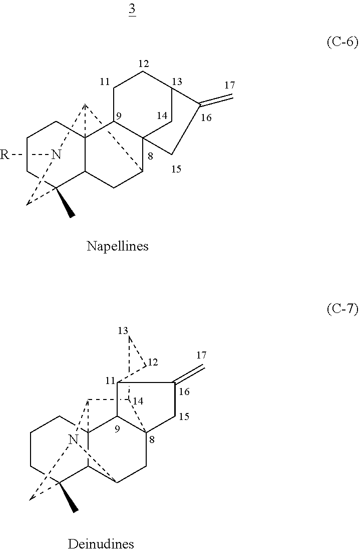

Examples of diterpenoid alkaloids that may be generated are described, for example, by Yin et al., RSC Advances 10 (23): 13669-13686 (2020); Nyirimigabo et al., J Pharm Pharmacol 67 (1): 1-19 (2015); Csupor et al., Journal of Chromatography 1216 (11), 2079-2086 (2009); and Zhou et al. J Ethnopharmacol 160: 173-193 (2015), each of which is incorporated herein by reference in its entirety. The diterpenoid alkaloids generated by the expression systems, enzymes and methods provided herein can have a wide range of applications from antifeedants to anti-cancer agents, choline esterase inhibitors, and analgesics.

### Enzymes

Seven enzymes have been identified from Siberian Larkspur (Delphinium grandiflorum) The biosynthetic pathway includes a pair of terpene synthases, four cytochrome P450s—three of which are the founding members of new subfamilies with one belonging to the poorly characterized CYP729 family—and a reductase with little homology to other characterized enzymes. P450 enzymes (P450s) are widely involved in biosynthetic pathway of plant natural products due to the wide range of their activities including hydroxylation, reduction, decarboxylation, sulfoxidation, N-demethylation and epoxidation, deamination, and dehalogenation. These enzymes and production of a key intermediate in a heterologous host paves the way for biosynthetic production of a group of metabolites such as diterpenoid alkaloids that are useful for medicinal applications.

The enzymes described herein can catalyze the following biosynthetic pathways.

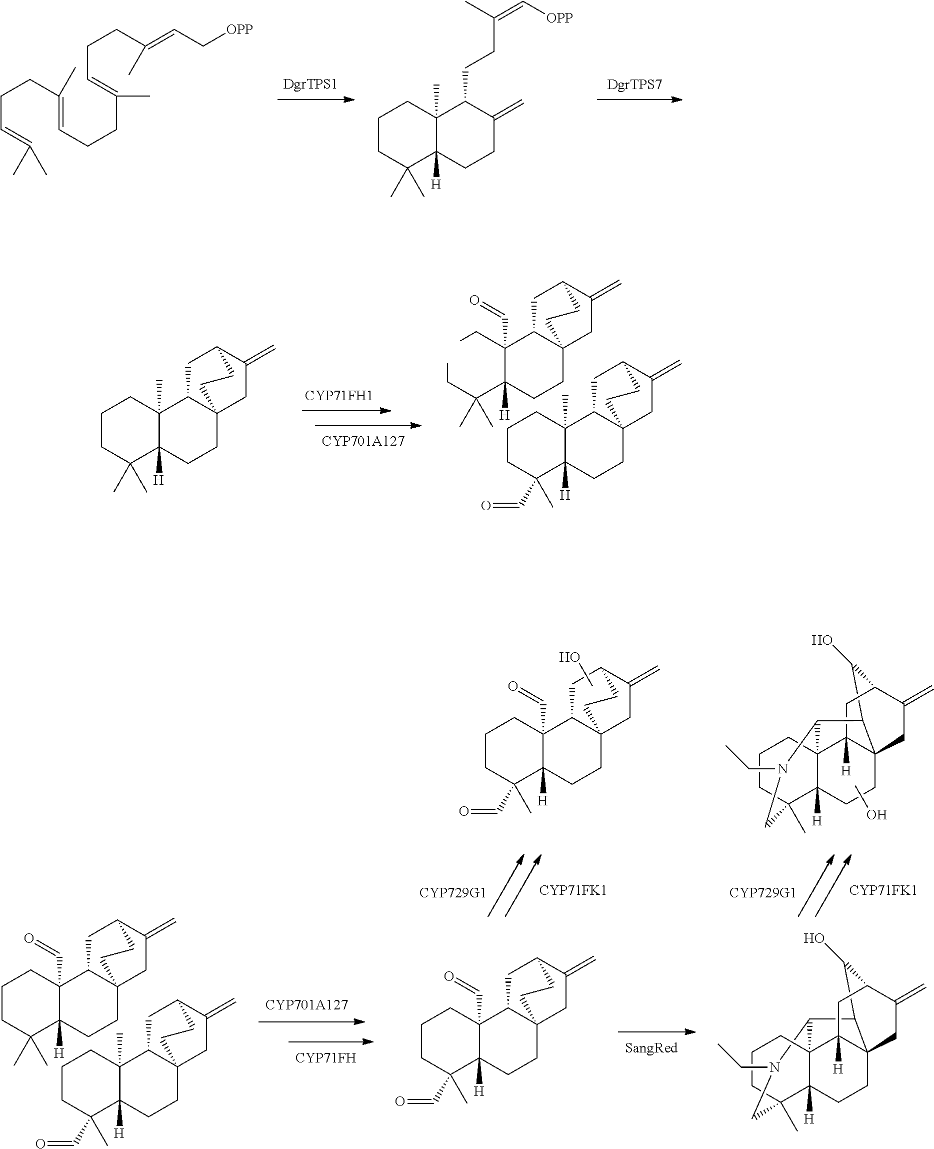

In an early step in the biosynthetic pathway, a first class II TPS can convert geranylgeranyl diphosphate (GGPP) to a copalyl diphosphate (CPP), shown to be an ent-CPP, and second a class I TPS converts ent-CPP to ent-atiserene. For example, GGPP can be converted to ent-CPP by Delphinium grandiflorum TPS1 (DgrTPS1) as illustrated below.

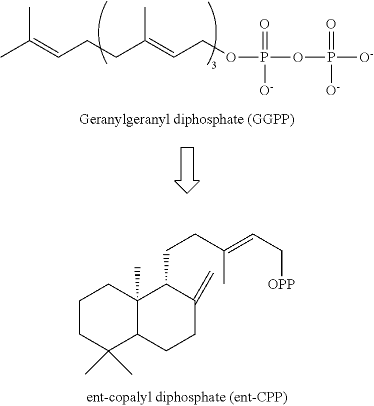

An amino acid sequence for the DgrTPS1 enzyme is shown below as SEQ ID NO:1.

| | |
|---|---|
| 1 | MASLSLHSAS SHLSASPAEV SPPLFSSGFA HSLPVKNKRD |
| 41 | DGHNSRCSAT SKHDGQVYKE VTKQDTIRKW QEITNQDSKN |
| 81 | GAVKVDDINK LAEWIGDIKN MLRSMDDGEI SVSAYDTAWV |
| 121 | ALVENIHGFY GPQFPSSVEW IVNNQLGDGS WGDEPIFSAH |
| 161 | DRILNTLGCV VALKTWSIHP EKCEKGLSYI RQNISRLDDE |
| 201 | STEHMPIGFE IAFPSLIEMA RKLNLDIPYD SAAVLAIYAQ |
| 241 | KDIKLMKIPM EKAHKWPTTL LHSLEGMDGL DWDKLMKLQS |
| 281 | SNGSFLFSPA STAFALMNTK DEKCLEYLKK PVEKENGGVP |
| 321 | NVYPVDLFEH IWVVDRLERL GVSRYFEAEI KDCIDYVAKY |
| 361 | WTKSGIAWAR NSTVCDIDDT AMGFRLLRLH GYNVSPDVFK |
| 401 | NFQNGDEFVC FAGQSNQAVT GMYNLYRAAQ VAFPGETILE |
| 441 | DCKKFSYKFL RNKQATNQLL DKWIITKDLP GEVGYALDFP |
| 461 | WYANLPRIET RLYLEQYGGD EDVWIGKTLY RMSYVNNGTY |
| 521 | LNAAKLDENN CQAVHHVEWD NIQKWYLECN LAEFGVTDAR |
| 561 | LLQTYFVATA SIFEPERSSE RLAWIKIALL LESILSHFKD |
| 601 | ETKEHRKAFI VDFIENKVVS RKLNYSTGKA SNLVHTLVGT |
| 641 | LQDIAITNGS GIQNALLDTF EKWLETWEIR FSSKEVAGLL |
| 681 | ANMINICSGN EVSDEVSSNP EYRSLVDLIN KICFQLGQAS |
| 721 | KVGINGTRVN GLEIPSVELD MEELVKIVVR KDNGIDSKVK |
| 761 | QTFLEVVKSF FYVSQCPKEV MERHIEEVLF NRVA |

A nucleotide sequence that encodes the DgrTPS1 enzyme of SEQ ID NO:1 is shown below as SEQ ID NO:2.

| | |
|---|---|
| 1 | ATGGCCTCTC TCTCCCTCCA CTCTGCTTCT TCCCACCTCT |
| 41 | CAGCATCACC TGCAGAGGTA TCACCTCCAC TGTTTTCATC |
| 81 | AGGATTTGCT CATTCACTTC CTGTTAAGAA TAAACGCGAT |
| 121 | GATGGTCACA ACTCAAGATG CTCTGCAACA TCGAAACATG |
| 161 | ATGGTCAAGT ATATAAAGAG GTTACGAAGC AGGATACGAT |
| 201 | AAGAAAATGG CAAGAAATTA CAAACCAAGA TAGCAAGAAC |
| 241 | GGCGCGGTTA AGGTTGATGA TATCAACAAG CTAGCAGAGT |
| 281 | GGATTGGAGA CATAAAAAAT ATGCTGCGTT CTATGGACGA |
| 521 | TGGGGAGATA AGCGTCTCGG CCTATGACAC GGCTTGGGTT |
| 561 | GCTCTGGTCG AAAACATTCA TGGCTTTTAT GGCCCTCAGT |
| 601 | TTCCGTCGAG TGTTGAATGG ATCGTTAATA ATCAGCTAGG |
| 641 | TGATGGTTCC TGGGGCGATG AGCCTATTTT CTCTGCACAT |
| 681 | GATCGGATAC TAAATACATT GGGCTGTGTG GTTGCGTTAA |
| 721 | AAACATGGAG CATTCATCCC GAGAAATGCG AGAAGGGATT |
| 761 | GTCGTATATC CGTCAGAACA TCAGCAGGCT GGATGATGAA |
| 801 | AGTACTGAAC ACATGCCTAT AGGGTTTGAG ATCGCCTTTC |
| 841 | CTTCTCTTAT CGAAATGGCA CGGAAGTTAA ACTTGGATAT |
| 881 | CCCCTATGAC TCGGCTGCAG TGCTCGCAAT ATACGCCCAA |
| 921 | AAGGATATAA AGCTCATGAA GATACCGATG GAGAAGGCAC |
| 961 | ATAAATGGCC CACTACGCTA CTTCACAGTT TGGAAGGCAT |
| 1001 | GGATGGATTG GATTGGGATA AACTTATGAA GTTGCAAAGC |
| 1041 | TCAAATGGCT CCTTCTTGTT CTCTCCAGCA TCGACGGCCT |
| 1081 | TCGCCCTTAT GAACACTAAA GATGAAAAGT GTCTTGAATA |
| 1121 | TCTCAAGAAA CCGGTTGAAA AATTCAATGG TGGAGTCCCG |
| 1161 | AATGTCTATC CTGTAGACTT GTTTGAACAT ATTTGGGTGG |
| 1201 | TTGATCGTTT GGAACGTCTT GGAGTTTCAC GCTACTTCGA |
| 1241 | GGCAGAAATC AAAGATTGCA TCGACTATGT AGCTAAATAT |
| 1281 | TGGACTAAAT CTGGGATAGC TTGGGCGAGA AACTCGACTG |
| 1321 | TTTGTGACAT AGATGACACG GCCATGGGGT TCAGGCTTCT |
| 1361 | ACGCCTACAT GGATACAACG TCTCCCCTGA TGTGTTTAAG |
| 1401 | AATTTTCAAA ACGGCGATGA GTTTGTTTGT TTTGCTGGAC |
| 1441 | AATCAAACCA GGCCGTTACA GGGATGTACA ATCTTTATAG |
| 1481 | GGCTGCTCAG GTGGCCTTCC CTGGGGAGAC TATCCTGGAA |
| 1521 | GATTGCAAGA AATTTTCCTA CAAATTTCTT CGCAATAAAC |
| 1561 | AAGCTACCAA CCAACTTTTA GATAAATGGA TCATAACAAA |
| 1601 | GGATTTGCCA GGGGAGGTTG GGTACGCCCT AGATTTTCCA |
| 1641 | TGGTATGCAA ACCTACCCCG AATCGAAACA CGCCTTTACT |
| 1681 | TGGAACAATA TGGTGGTGAT GAAGACGTCT GGATAGGGAA |
| 1721 | AACGCTTTAC AGGATGTCGT ATGTTAACAA TGGCACATAT |
| 1761 | CTTAACGCGG CCAAACTAGA CTTCAATAAT TGTCAAGCAG |
| 1801 | TCCATCATGT TGAATGGGAT AATATCCAAA AGTGGTACCT |
| 1841 | TGAGTGCAAT CTAGCTGAGT TCGGAGTGAC CGATGCAAGA |
| 1881 | CTTCTACAAA CTTATTTTGT AGCTACTGCA AGCATATTTG |
| 1921 | AGCCTGAAAG ATCGTCTGAG AGGCTTGCAT GGACCAAGAT |
| 1961 | TGCTTTGCTC CTCGAGTCAA TTTTGTCACA CTTCAAAGAT |
| 2001 | GAAACCAAGG AACACCGAAA GGCGTTTATC GTCGACTTTA |
| 2041 | TTGAGAATAA GGTTGTATCA AGGAAATTGA ACTACTCCAC |
| 2081 | TGGCAAGGCA AGCAATCTTG TGCATACTCT TGTTGGGACC |
| 2121 | TTACAAGATA TCGCAATAAC CAATGGAAGC GGCATTCAGA |
| 2161 | ACGCACTACT TGATACTTTT GAGAAGTGGT TGTTTACTTG |
| 2201 | GGAAATCCGG TTTTCTTCAA AAGAAGTAGC GGGACTTTTG |
| 2241 | GCCAACATGA TAAACATATG CAGTGGAAAT GAAGTTTCTG |
| 2281 | ATGAGGTTTC ATCCAATCCT GAATATCGAA GTCTTGTCGA |
| 2321 | CTTGACCAAT AAAATCTGCT TCCAACTTGG TCAGGCTAGT |
| 2361 | AAGGTTGGGA TAAACGGCAC ACGAGTGAAT GGCTTGGAGA |
| 2401 | TACCATCGGT TGAACTCGAT ATGGAGGAGC TAGTGAAGAT |
| 2441 | TGTTGTTAGG AAGGACAATG GAATCGACAG TAAGGTCAAG |
| 2481 | CAGACGTTCC TCGAAGTTGT GAAAAGCTTC TTCTATGTCT |
| 2521 | CTCAGTGTCC AAAAGAAGTG ATGGAGCGTC ACATCGAAGA |
| 2561 | AGTCCTCTTC AACCGAGTAG CCTAA |

The Delphinium grandiflorum TPS7a and TPS7b (DgrTPS7a and DgrTPS7b) enzymes can both convert ent-CPP to ent-atiserene. This reaction is shown below.

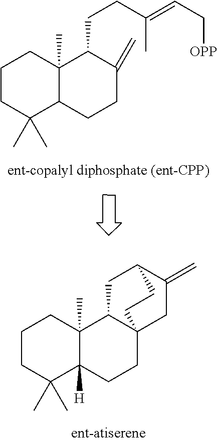

An amino acid sequence for the DgrTPS7a enzyme is shown below as SEQ ID NO:3.

| | |
|---|---|
| 1 | MYLSHPTKSP LVFPNPTTSS PRGSSSTSIS AVSVDHGVKR |
| 41 | LEKSENSLKI SEATKEKISK IFTKVELSKS SYDTAWVAMV |
| 81 | PSLDSSASPY FPECLNWILE NQHTDGSWGL TQQHPLLLKD |
| 121 | TLSSTLASIL ALKRWNVGED HVNKGLHFIS SNFASATDEK |
| 161 | QRCPIGFDII FPGMIERAQE IGVNFHLDPT SLNSILSKRD |
| 201 | TELHRVSTSN SEGSKLYRAY FAEGLRKSQN WEEVMKYQRK |
| 241 | NGSLENSPST TAVAAAHVQD PNCFKYLHSI LEEFGNAVPT |
| 281 | SYPLDIYTQL CMIDALEKLG ISRHFKNEVG NVLDKTYSSW |
| 321 | LTKDEEIFLD VSTSAMAFRI LRVHGYDVSP DVLAQFGQEG |
| 361 | FSNILGGYLN DSGAVLEIYR ASQIVLPNEV FLEEQKSWSS |
| 401 | AYLKNELSKG SMHADRMHEW ISKEVETALT YPYKPNLPRL |
| 441 | EHRRIVEHYN VDNLRVLKSA YRPLGIDNKD LLHLAMEDEN |
| 481 | ICQSIYQNEF KELERWVKDN RIDKLKFARQ KQVYTLFSSA |
| 521 | STLFPPELSD ARLSWAKFSI LITIIDDCYD LGGSRDELIN |
| 561 | LNQVFDKWDG VTAGDFISEP VEILYYAYKN TIDDLARKAF |
| 601 | KYQHRDITKH LVENCVEMVK SMWIEAEWME HNVVPSLEEY |
| 641 | NENGYVSFAL GPIVLTTLYF VGPQLSEEVV RSSEYHDLER |
| 681 | LMSTICRNLN DLRIVQKELS EGTINGVSIL MIHDPEVKTE |
| 721 | EDSVKKIREA IEICEKELIK LVLRRKDCVV PRACKELFWN |
| 761 | MIRINNLFYA SIDGYTSETQ MMNEVKAVMR IPLTRPDLIE |
| 801 | G |

A nucleotide sequence that encodes the DgrTPS7a enzyme of SEQ ID NO:3 is shown below as SEQ ID NO:4.

| | |
|---|---|
| 1 | ATGTATCTCT CCCATCCAAC CAAGTCGCCT CTCGTCTTTC |
| 41 | CGAACCCAAC AACATCATCG CCGAGGGGAT CCTCCTCCAC |
| 81 | ATCCATCTCA GCTGTTTCTG TGGATCATGG TGTTAAGAGG |
| 121 | TTGGAAAAAT CTGAAAATTC TCTTAAGATT TCCGAGGCGA |
| 161 | CCAAGGAGAA AATAAGCAAA ATCTTCACCA AGGTTGAGCT |
| 201 | TTCGAAATCT TCATACGACA CCGCTTGGGT TGCAATGGTC |
| 241 | CCTTCTCTTG ACTCCTCTGC ATCGCCCTAC TTTCCCGAAT |
| 281 | GTCTCAACTG GATCTTGGAG AATCAACACA CGGACGGCTC |
| 321 | ATGGGGCCTT ACTCAGCAAC ACCCTTTATT GTTAAAGGAC |
| 361 | ACGCTGTCGT CGACATTAGC CTCTATACTT GCACTCAAAA |
| 401 | GATGGAATGT CGGCGAAGAC CATGTTAACA AGGGTCTCCA |
| 441 | TTTCATTAGT TCTAATTTTG CTTCCGCCAC AGACGAGAAG |
| 481 | CAACGTTGTC CAATTGGGTT TGACATCATA TTCCCCGGTA |
| 521 | TGATCGAGCG TGCTCAGGAG ATAGGAGTAA ACTTCCATTT |
| 561 | AGACCCAACG AGTTTAAATT CTATTCTTAG TAAGAGAGAC |
| 601 | ACGGAATTAC ATAGGGTATC TACAAGCAAC TCAGAGGGAA |
| 641 | GCAAACTCTA CCGAGCCTAC TTTGCGGAGG GACTGAGGAA |
| 681 | ATCGCAAAAT TGGGAGGAAG TAATGAAATA TCAGAGAAAG |
| 721 | AATGGATCGT TGTTTAACTC TCCTTCCACC ACTGCGGTCG |
| 761 | CGGCGGCTCA CGTTCAAGAC CCGAATTGCT TCAAGTACTT |
| 801 | GCACTCGATC TTGGAGGAAT TCGGCAATGC AGTCCCGACT |
| 841 | AGTTATCCAC TAGACATATA CACCCAGCTC TGTATGATTG |
| 861 | ACGCTCTAGA GAAACTGGGA ATCTCCCGAC ACTTCAAGAA |
| 921 | TGAGGTAGGA AATGTTTTGG ATAAAACCTA CAGTTCCTGG |
| 961 | CTGACCAAGG ATGAGGAAAT CTTTTTAGAC GTTTCAACAT |
| 1001 | CGGCCATGGC ATTTAGGATA TTACGTGTAC ATGGATACGA |
| 1041 | CGTCTCCCCA GACGTACTAG CTCAATTCGG CCAAGAAGGT |
| 1081 | TTCTCAAATA CACTTGGAGG ATACCTAAAC GACTCAGGGG |
| 1121 | CTGTCCTTGA GATATATCGG GCGTCCCAAA TTGTGCTCCC |
| 1161 | CAATGAGGTA TTTCTGGAGG AACAAAAATC TTGGTCAAGT |
| 1201 | GCTTATCTTA AGAATGAACT ATCCAAGGGT TCGATGCACG |
| 1241 | CCGATAGAAT GCATGAATGG ATTAGCAAAG AGGTCGAAAC |
| 1281 | GGCGCTTACC TATCCCTACA AACCCAATTT GCCGCGCTTA |
| 1321 | GAGCACAGGA GAACCGTGGA ACATTACAAT GTCGATAACT |
| 1361 | TGAGAGTTCT GAAATCAGCA TATAGGCCTC TTGGTATTGA |
| 1401 | CAACAAGGAT TTACTGCATT TGGCGATGGA AGATTTTAAT |
| 1441 | ATTTGTCAAT CGATATATCA AAATGAATTC AAGGAGCTCG |
| 1481 | AGAGGTGGGT GAAAGACAAC AGGATAGATA AGCTAAAGTT |
| 1521 | CGCAAGGCAA AAGCAGGTGT ACACGCTCTT CTCTTCCGCA |
| 1561 | TCAACTCTAT TTCCTCCAGA ATTAAGTGAC GCGCGTCTCT |
| 1601 | CGTGGGCAAA GTTCAGTATC CTCACAACTA TAATTGACGA |
| 1641 | TTGCTACGAT TTAGGCGGCT CTAGAGACGA ACTAATTAAC |
| 1681 | CTAAACCAAG TGTTTGACAA GTGGGATGGA GTTACAGCCG |
| 1721 | GTGACTTCAT TTCCGAGCCA GTTGAAATAC TATATTATGC |
| 1761 | ATACAAAAAT ACGATTGATG ATCTTGCAAG AAAGGCTTTC |
| 1801 | AAATATCAGC ATCGGGATAT CACAAAGCAT TTAGTGGAGA |
| 1841 | ACTGTGTTGA AATGGTTAAG TCTATGTGGA TCGAGGCAGA |
| 1881 | GTGGATGGAG CACAATGTAG TACCATCACT GGAAGAATAC |
| 1921 | AATGAAAATG GATACGTATC GTTTGCTCTG GGGCCTATAG |
| 2001 | TTCTTACAAC TTTATATTTT GTTGGGCCCC AACTTTCCGA |
| 2041 | GGAAGTCGTA AGGAGTTCTG AGTACCATGA CCTATTTCGA |
| 2081 | CTCATGAGCA CAATATGTCG TAACCTCAAT GATCTTCGAA |
| 2121 | CAGTTCAGAA GGAACTAAGC GAAGGGACGA TAAACGGTGT |
| 2161 | GTCCATTCTG ATGATACACG ACCCTGAAGT CAAGACGGAG |
| 2201 | GAAGACTCGG TGAAAAAGAT TAGAGAAGCG ATTGAGATTT |
| 2241 | GCGAGAAGGA ACTGATAAAA CTAGTGTTGC GGAGGAAGGA |
| 2281 | CTGCGTGGTA CCTAGAGCTT GCAAAGAGTT GTTTTGGAAT |
| 2321 | ATGATCAGAA TAAACAACCT GTTTTACGCG AGCATTGATG |
| 2361 | GCTACACGTC TGAAACCCAA ATGATGAATG AGGTGAAGGC |
| 2401 | TGTCATGCGC ATTCCCCTCA CTAGACCAGA CTTAATTGAA |
| 2441 | GGTTAG |

An amino acid sequence for the DgrTPS7b enzyme is shown below as SEQ ID NO:5.

| | |
|---|---|
| 1 | MYLSHPTKSP LVFPNPTTSS PRRSSSTSIS AVSVDHGVKR |
| 41 | LEKSENSLKI SEESKEKISK IFTKVELSKS SYDTAWVAMV |
| 81 | PSLDSSVSPY FPECLNWILE NQHADGSWGL TQQHPLLLKD |
| 121 | TLSSTLASIL ALKRWNVGED HVNKGLHFIS SNFASATDEK |
| 161 | QRSPIGFDII FPGMIEHAQE IGVNFHLDPT SLNSIISKRD |
| 201 | MELHRVSTSN SEGSKLYRAY FAEGLRKSQN WEEVMKYQRK |
| 241 | NGSLENSPST TAVAAAHVQD PNCLKYLHSI LEEFGNAVPT |
| 281 | SYPLDIYTQL CMIDALEKLG ISRHFKNEII NVLDKTYGSW |
| 321 | LTKDEEIFLD VSTSAMAFRI LRVHGYDVSP DVLAQFDQQG |
| 361 | FSNTLGGYLN DSGAVLEIYR ASQIVLPDEV FLEEQKTWSS |
| 401 | AYLKNELSKG SMHADRMHEW ISKEVETALT YPYKPNLPRL |
| 441 | EHRRTVEHYN VDNLRVLKSA YRPLGIDNKD LLHLAMEDEN |
| 481 | LCQSIYQNEF KELERWVKDN RIDKLKFARQ KQVYTLFSSA |
| 521 | STLFPPELSD ARLSWAKFSI LTTIIDDCYD LGGSRDELIN |
| 561 | LNQVFDKWDG VIAGDFISEP VEILYYAYKN TIDDLARKAF |
| 601 | KYQHRDITKH LVENCVEMVK SMWIEAEWME HNVVPSLEEY |
| 641 | NENGYVSFAL GPIVLITLYF VGPQLSEEVV RSSEYHDLFR |
| 681 | LMSTICRNLN DLRTVQKELS EGTINGVSIL MIHDPEVKTE |
| 721 | EDSVKKIREA IEICEKELIK LVLPRKDCVV PRACKELFWN |
| 761 | MIRINNLFYA SIDGYTSETQ MMNEVKAVMR IPLTRPDLIE |
| 801 | G |

A nucleotide sequence that encodes the DgrTPS7b enzyme of SEQ ID NO:5 is shown below as SEQ ID NO:6.

| | |
|---|---|
| 1 | ATGTATCTCT CCCATCCAAC CAAGTCGCCT CTCGTCTTTC |
| 41 | CGAACCCAAC AACATCATCG CCGAGGAGAT CCTCCTCCAC |
| 81 | ATCCATCTCA GCTGTTTCTG TGGATCATGG TGTTAAGAGG |
| 121 | TTGGAAAAAT CTGAAAATTC TCTTAAGATT TCCGAGGAGA |
| 161 | GCAAGGAGAA AATAAGCAAA ATCTTCACCA AGGTTGAACT |
| 201 | TTCGAAATCT TCATACGACA CCGCTTGGGT TGCAATGGTC |
| 241 | CCTTCTCTTG ACTCCTCTGT ATCACCCTAC TTTCCCGAAT |
| 281 | GTCTCAACTG GATCTTGGAG AATCAACACG CGGACGGCTC |
| 321 | ATGGGGCCTT ACTCAGCAAC ACCCTTTATT GTTAAAGGAC |
| 361 | ACGCTGTCGT CGACATTGGC CTCTATACTC GCACTCAAAA |
| 401 | GATGGAATGT CGGCGAAGAC CATGTGAACA AGGGTCTCCA |
| 441 | TTTCATTAGT TCTAATTTTG CTTCCGCCAC GGACGAGAAG |
| 481 | CAACGTAGTC CAATTGGGTT TGACATCATA TTCCCCGGTA |
| 521 | TGATCGAGCA TGCCCAGGAG ATAGGAGTAA ACTTCCATTT |
| 561 | AGACCCAACG AGTTTAAATT CTATTATTAG TAAGAGAGAC |
| 601 | ATGGAATTAC ATAGGGTATC TACAAGCAAC TCAGAGGGGA |
| 641 | GCAAACTCTA CCGAGCCTAC TTTGCGGAGG GACTGAGGAA |
| 681 | GTCGCAAAAT TGGGAGGAAG TAATGAAATA TCAGAGAAAG |
| 721 | AATGGATCGT TGTTTAATTC TCCTTCCACC ACTGCGGTTG |
| 761 | CGGCCGCTCA CGTCCAAGAC CCGAATTGCT TGAAGTACTT |
| 801 | GCACTCGATC TTGGAGGAAT TCGGCAATGC AGTCCCGACT |
| 881 | AGTTATCCAC TAGACATATA CACCCAGCTC TGTATGATTG |
| 921 | ACGCTCTAGA GAAACTGGGA ATCTCCCGAC ACTTCAAGAA |
| 961 | TGAGATAATA AATGTTTTGG ATAAAACCTA CGGTTCCTGG |
| 1001 | TTGACCAAGG ACGAGGAAAT CTTTTTAGAC GTTTCGACAT |
| 1041 | CTGCCATGGC ATTTAGGATA TTACGTGTAC ATGGATATGA |
| 1081 | CGTCTCCCCA GACGTACTAG CTCAATTCGA CCAACAAGGT |
| 1121 | TTCTCAAATA CACTTGGAGG ATATCTAAAC GACTCAGGGG |
| 1161 | CTGTCCTTGA GATATATCGG GCGTCCCAAA TTGTGCTCCC |
| 1201 | CGATGAGGTA TTTCTGGAGG AACAAAAAAC TTGGTCAAGT |
| 1241 | GCTTATCTTA AGAATGAACT ATCCAAGGGT TCGATGCACG |
| 1281 | CCGATAGAAT GCATGAATGG ATTAGCAAAG AGGTCGAAAC |
| 1321 | GGCGCTAACC TATCCCTACA AACCCAATTT GCCGCGCTTA |
| 1361 | GAGCACAGGA GAACCGTGGA ACATTACAAT GTCGATAACT |
| 1401 | TGAGAGTTCT GAAATCAGCA TATAGGCCTC TTGGTATTGA |
| 1481 | CAACAAGGAT TTACTGCATT TGGCGATGGA AGACTTTAAT |
| 1521 | CTTTGTCAAT CGATATATCA AAATGAATTC AAGGAGCTCG |
| 1561 | AGAGGTGGGT GAAAGACAAC AGGATAGATA AGCTAAAGTT |
| 1601 | CGCAAGGCAA AAGCAGGTGT ACACGCTCTT CTCTTCCGCA |
| 1641 | TCAACTCTAT TTCCTCCAGA ATTAAGTGAC GCGCGTCTCT |
| 1681 | CGTGGGCAAA GTTCAGTATC CTCACAACTA TAATTGACGA |
| 1721 | TTGCTACGAT TTAGGCGGCT CTAGAGACGA ACTAATTAAC |
| 1761 | CTAAACCAAG TGTTTGACAA GTGGGATGGA GTTACAGCCG |
| 1801 | GTGACTTCAT TTCCGAGCCA GTTGAAATAC TATATTATGC |
| 1841 | ATACAAAAAT ACGATTGATG ATCTTGCAAG AAAGGCTTTC |
| 1881 | AAATATCAGC ATCGGGATAT CACAAAGCAT TTAGTGGAGA |
| 1921 | ACTGTGTTGA AATGGTTAAG TCTATGTGGA TCGAGGCAGA |
| 1961 | GTGGATGGAG CACAATGTAG TACCATCACT GGAAGAATAC |
| 2001 | AATGAAAATG GATACGTATC GTTTGCTCTG GGGCCTATAG |
| 2041 | TTCTTACAAC TTTATATTTT GTTGGGCCCC AACTTTCCGA |
| 2081 | GGAAGTCGTA AGGAGTTCTG AGTACCATGA CCTATTTCGA |
| 2121 | CTCATGAGCA CAATATGTCG TAACCTCAAT GATCTTCGAA |
| 2161 | CAGTTCAGAA GGAACTAAGC GAAGGGACGA TAAACGGTGT |
| 2201 | GTCCATTCTG ATGATACACG ACCCTGAAGT CAAGACGGAG |
| 2241 | GAAGACTCGG TGAAAAAGAT TAGAGAAGCG ATTGAGATTT |
| 2281 | GCGAGAAGGA ACTGATAAAA CTAGTGTTGC CGAGGAAGGA |
| 2321 | CTGCGTGGTA CCTAGAGCTT GCAAAGAGTT GTTTTGGAAT |
| 2361 | ATGATCAGAA TAAACAACCT GTTTTACGCG AGCATTGATG |
| 2401 | GCTACACGTC TGAAACCCAA ATGATGAATG AGGTGAAGGC |
| 2441 | TGTCATGCGC ATTCCCCTCA CTAGACCAGA CTTAATTGAA |
| 2481 | GGTTAG |

As illustrated herein, the Delphinium grandiflorum CYP701A127 and CYP71FH1 enzymes both showed oxidizing activity, for example in oxidizing the ent-atiserene backbone to generate one or more types of aldehydes. For example, the oxidation of ent-atiserene to ent-atiserene-19-al can be catalyzed by Delphinium grandiflorum CYP701A127 and/or Delphinium grandiflorum CYP71FH1 as shown below.

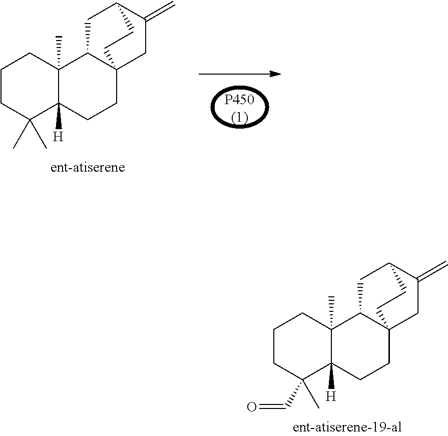

An amino acid sequence for the Delphinium grandiflorum CYP701A127 enzyme is shown below as SEQ ID NO:7.

| | |
|---|---|
| 1 | MAITKEILQQ LTPQTITITV VLGLFVLILL RIKKSPINSA |
| 41 | LPSLPVVPGL PLIGNLHQLS DKKPHQTFTK WAEKYGPIYS |
| 81 | IKTGSSTLVV LNSNDVAKEA MVTRESSIST RKLSNALTIL |
| 121 | TLDKKIVAIS DYGDFHKITK KYLISGMLGA NAQKRYRGHR |
| 161 | ETMMSNMLSK LCAHIKEKPL ESVNLRSIFQ YELFGLALKQ |
| 201 | AYGRDLDAPF YIEGLGTKLS RYEIFEALVV DPMMGAIAVD |
| 241 | WRDFFPYLRW IPNKGLEARI ERMAFRRKAV CKALIDAQKR |
| 281 | RRATGEILDS YVDYLLAPDL KQFSEDELIM LMWEVVIETS |
| 321 | DTTLVTTEWA MYEIAKNRRV QELLYRELKE VCGSEKVTED |
| 361 | HLPRLPYLNA VFHETLRRHS PAPMIPLRYV HEDTELGGYH |
| 401 | IPAGTQISIN IFGCNMDKKQ WDEPEAWKPE RFLDPKEDPT |
| 441 | DMFKSMAFGG GKRICAGAQQ AMTIACMAIA TYVQEFDWKL |
| 481 | DEGQKEDVNT LGLISYRLYP LQVHIKPRTA |

A nucleotide sequence that encodes the Delphinium grandiflorum CYP701A127 enzyme of SEQ ID NO:7 is shown below as SEQ ID NO:8.

| | |
|---|---|
| 1 | ATGGCCATTA CCAAAGAGAT CCTTCAACAG TTAACCCCTC |
| 41 | AAACTATTAC CATCACTGTA GTTTTGGGCC TCTTTGTACT |
| 81 | CATCTTGCTC AGAATCAAGA AATCTCCTAC AAACTCAGCT |
| 121 | CTACCTTCTC TACCTGTTGT TCCTGGGCTC CCTTTGATTG |
| 161 | GGAATTTGCA CCAACTGAGT GATAAGAAGC CACACCAGAC |
| 201 | TTTCACAAAG TGGGCAGAGA AATATGGACC TATTTATTCC |
| 241 | ATTAAGACTG GTTCTTCTAC TCTTGTTGTC CTCAACTCAA |
| 281 | ATGATGTGGC TAAAGAGGCT ATGGTGACTA GATTCTCATC |
| 321 | TATCTCCACA AGGAAGCTCT CCAATGCTTT GACGATACTC |
| 361 | ACACTCGATA AAAAGATTGT TGCCATAAGT GACTACGGGG |
| 401 | ATTTCCACAA GATCACTAAG AAGTATCTGA TTTCGGGCAT |
| 441 | GCTAGGTGCC AACGCGCAGA AGCGATATCG AGGTCATAGA |
| 481 | GAAACCATGA TGAGTAATAT GTTGAGTAAG TTATGTGCTC |
| 521 | ACATCAAGGA AAAGCCTCTT GAATCTGTAA ACTTAAGAAG |
| 561 | TATATTTCAG TATGAACTCT TTGGATTAGC TCTGAAACAA |
| 601 | GCTTATGGTA GAGATTTAGA CGCCCCGTTT TATATTGAAG |
| 641 | GTCTTGGTAC AAAATTGTCA AGATATGAGA TATTTGAGGC |
| 681 | GTTAGTCGTC GATCCAATGA TGGGAGCAAT TGCTGTGGAC |
| 721 | TGGAGAGACT TTTTCCCATA TTTGAGATGG ATTCCAAACA |
| 761 | AAGGGCTGGA AGCAAGGATT GAGCGAATGG CTTTCCGGAG |
| 801 | AAAAGCTGTG TGTAAAGCGC TCATAGATGC ACAAAAGAGA |
| 841 | CGAAGAGCTA CTGGAGAGAT ATTAGACAGT TATGTGGATT |
| 881 | ACTTGTTAGC CCCGGACCTA AAGCAGTTCT CAGAGGATGA |
| 921 | ACTGATCATG TTAATGTGGG AAGTGGTTAT TGAGACCTCA |
| 961 | GACACCACTT TGGTCACTAC AGAATGGGCT ATGTATGAAA |
| 1001 | TCGCAAAGAA CAGGAGAGTT CAGGAACTCC TCTACCGGGA |
| 1041 | GCTTAAAGAG GTTTGTGGAT CTGAGAAGGT TACTGAGGAT |
| 1081 | CATTTGCCAA GGCTACCATA CTTGAACGCC GTCTTCCATG |
| 1121 | AAACTTTGAG AAGACATTCT CCAGCTCCAA TGATCCCACT |
| 1161 | AAGATACGTA CATGAAGATA CCGAATTGGG AGGCTACCAC |
| 1201 | ATCCCAGCTG GAACTCAGAT CTCCATAAAC ATCTTTGGAT |
| 1241 | GCAACATGGA CAAGAAGCAA TGGGACGAAC CGGAAGCTTG |
| 1281 | GAAGCCCGAG AGGTTCCTAG ACCCCAAATT TGATCCAACT |
| 1321 | GATATGTTCA AGTCAATGGC TTTCGGGGGA GGCAAGAGAA |
| 1361 | TATGTGCAGG AGCGCAACAG GCCATGACGA TTGCTTGCAT |
| 1401 | GGCGATTGCT ACGTACGTGC AGGAGTTTGA TTGGAAGTTG |
| 1441 | GATGAAGGAC AGAAAGAGGA TGTTAATACT CTTGGACTGA |
| 1481 | CCAGTTACAG ACTCTATCCT CTCCAGGTGC ACATAAAACC |
| 1521 | AAGAACAGCT TAA |

An amino acid sequence for the Delphinium grandiflorum CYP71FH1 enzyme is shown below as SEQ ID NO:9.

| | |
|---|---|
| 1 | MAQLQPLLQW LETQQETLER HPAALILVSI FTTLLLVRLM |
| 41 | SGFWSKKSNM YLLPSPPTLP IIGNFHQLTT LPHRGLFKLS |
| 81 | NKYGHLMLLH LGRAPAVIVS SAEMAREIKK THDVAFANRP |
| 121 | YSIASEILFY GRSNMAFAPY GEYWRQVRKI CNLELLSLKR |
| 161 | VQTFKYVREE EVAILIKTVK EASKTKLPMN LTENLLGLIN |
| 201 | NIVSRCALGK KSRGEGSNMK LGVLSRQFIQ MLEAFSFKDH |
| 241 | FPILGFLDHV TGLYRKMKYV SGELDAFLEE TIDEHEAQKT |
| 281 | QDYHEDREDF VDLLLRVKRD NTLDMDFTRK HIKALVLDMY |
| 321 | LGGTDTSSTT IEWTMTELLR HPFAMKKAQE EIRRVVGNKP |
| 361 | QVEEDDVNHM DYLKCALKET LRLHAPVPLI YLESSVNTDI |
| 401 | KGVKVPAKTK VIVNIWAIQR DGKSWDNPEE FIPERFMNNP |
| 441 | VDFRGQDYEY IPFGSGRRGC PGMTFGLSMV EYILANILYC |
| 481 | FDWNLPAGMT IADIDMDESF GSTVSKKDPL MLIPTLKPTN |

A nucleotide sequence that encodes the Delphinium grandiflorum CYP71FH1 enzyme of SEQ ID NO:9 is shown below as SEQ ID NO:10.

| | |
|---|---|
| 1 | ATGGCTCAGT TGCAACCATT GCTGCAATGG CTAGAAACCC |
| 41 | AGCAAGAAAC CCTGTTTCGC CATCCCGCGG CTCTCATTCT |
| 81 | TGTCTCCATC TTCACCACTC TCCTTCTAGT GAGGCTTATG |
| 121 | AGTGGCTTTT GGTCTAAAAA GTCCAATATG TACCTCCTTC |
| 161 | CATCACCTCC AACTCTCCCG ATCATCGGAA ATTTCCACCA |
| 201 | ACTCACCACA CTTCCTCACC GTGGTCTGTT TAAACTCTCC |
| 241 | AACAAGTACG GTCACCTGAT GCTTCTTCAT TTGGGGCGTG |
| 281 | CGCCCGCCGT GATAGTCTCC TCGGCCGAGA TGGCCAGAGA |
| 321 | GATCAAGAAA ACCCACGACG TGGCGTTTGC CAACAGGCCT |
| 361 | TACTCCATAG CCAGTGAGAT TCTCTTCTAC GGGCGCAGCA |
| 401 | ACATGGCGTT TGCCCCGTAC GGGGAATACT GGAGGCAGGT |
| 441 | CAGAAAGATA TGTAACTTGG AACTCTTGAG TTTGAAGAGA |
| 481 | GTTCAGACTT TTAAGTACGT AAGGGAGGAA GAGGTGGCGA |
| 521 | TTCTGATCAA GACTGTAAAA GAGGCTTCGA AGACAAAACT |
| 561 | CCCGATGAAC CTAACCGAGA ATCTACTCGG ACTCACCAAC |
| 601 | AACATAGTGT CGAGGTGCGC TCTTGGGAAG AAAAGCCGGG |
| 641 | GAGAAGGCAG TAACATGAAA TTAGGGGTGT TGTCAAGACA |
| 681 | GTTCATCCAG ATGTTGGAAG CTTTCAGCTT CAAAGACCAT |
| 721 | TTTCCAATCT TGGGGTTTTT GGATCACGTG ACCGGGTTGT |
| 761 | ACCGAAAGAT GAAATATGTT TCTGGAGAGC TGGACGCTTT |
| 801 | TCTCGAGGAA ACTATCGACG AACACGAAGC GCAGAAGACG |
| 841 | CAAGATTATC ACGAGGATAG AGAAGACTTT GTTGATCTCC |
| 881 | TACTGAGGGT GAAAAGAGAC AACACCCTAG ACATGGATTT |
| 921 | CACTAGGAAA CACATCAAAG CTCTAGTTCT GGACATGTAT |
| 961 | CTTGGGGGAA CAGACACTTC ATCAACCACC ATAGAATGGA |
| 1001 | CTATGACGGA GCTGCTGAGG CATCCGTTTG CGATGAAAAA |
| 1041 | AGCCCAAGAA GAGATCAGAA GAGTGGTTGG GAACAAGCCC |
| 1081 | CAGGTGGAAG AGGACGACGT CAATCATATG GACTACCTAA |
| 1121 | AATGCGCCCT CAAAGAAACC CTTCGCCTAC ATGCACCCGT |
| 1161 | GCCCTTGATC TACCTCGAGT CCTCGGTCAA TACCGATATA |
| 1201 | AAGGGAGTTA AAGTCCCAGC CAAAACAAAA GTGATAGTGA |
| 1241 | ACATATGGGC AATTCAAAGG GACGGAAAAT CGTGGGACAA |
| 1281 | TCCGGAAGAA TTCATCCCAG AAAGGTTTAT GAACAATCCG |
| 1321 | GTTGATTTCA GAGGGCAGGA TTATGAGTAC ATCCCGTTCG |
| 1361 | GGTCGGGACG AAGAGGCTGC CCGGGTATGA CATTCGGTCT |
| 1401 | GTCTATGGTA GAGTATATTT TGGCAAATAT ACTCTACTGT |
| 1441 | TTCGACTGGA ATCTGCCTGC TGGGATGACC ATAGCCGATA |
| 1481 | TCGACATGGA TGAAAGTTTC GGTAGCACTG TCAGTAAAAA |
| 1521 | AGATCCTCTC ATGCTCATTC CAACCCTCAA ACCTACCAAT |
| 1561 | TAG |

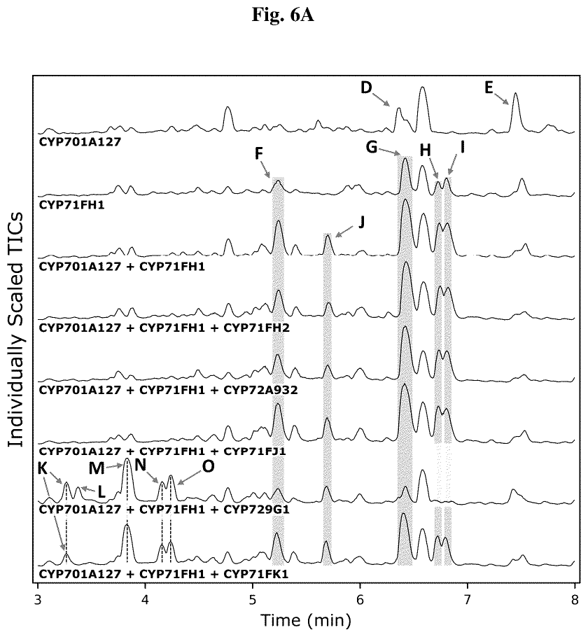

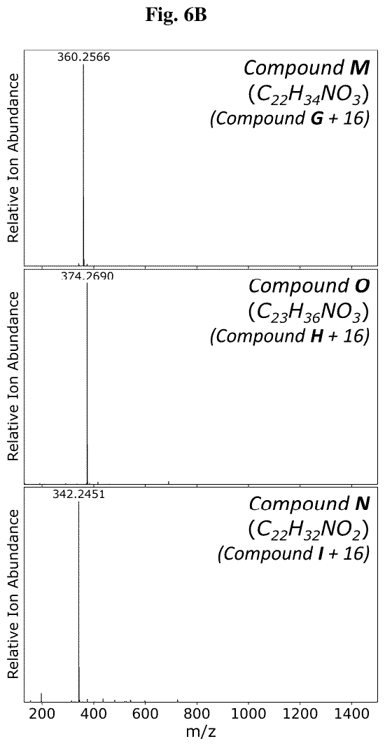

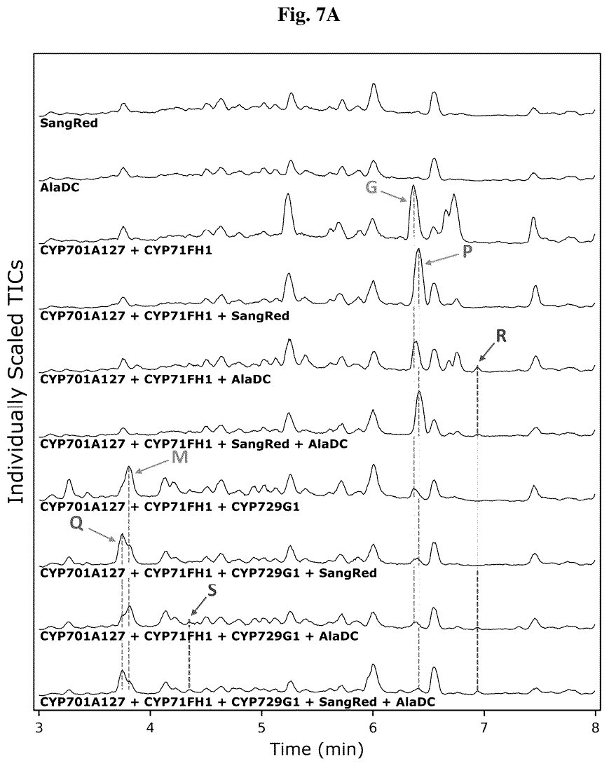

The Delphinium grandiflorum CYP729G1 and Delphinium grandiflorum CYP71FK1 enzymes can act on the products produced by the DgrTPS1, DgrTPS7, DgrCYP701A127, and DgrCYP71FH1. Results described herein show that DgrCYP729G1 and Dgr CYP71FK1 enzymes have similar functions but the Delphinium grandiflorum CYP729G1 enzyme generates compound L, as shown in FIG. 6A. Compound L has a molecular weight of 376.2501.

An amino acid sequence for the Delphinium grandiflorum CYP729G1 enzyme is shown below as SEQ ID NO:11.

| | |
|---|---|
| 1 | MELTQAQAWW SALVETILPF LVWLVESWNE LRYVKTQSSD |
| 41 | GGKLPPGHLG LPVIGQLLSF IWYFRIRRNP DDFVHSMRKR |
| 81 | YGDADGIYRS YLFGSPAIIG CSPDFNKFVL QSSNLFQATR |
| 121 | RQKDIFGHNS VAVVNGKAHY RLRGYINNTI STPDALKKIT |
| 161 | ICIQPNIVSS LQSWAEKGKI KGVYDIKKVF FETICIIITS |
| 201 | FKPGPAIDML DQHFHAILDG LGEKGTKFHL AVQSKKTLTE |
| 241 | VFKKEIDKRT QHGIPSEDQN DLMERLMRMR DEDGEPLSDD |
| 281 | EVIDNIVTCI MGGYESPFQL AIWALYFLAK NNDVLQKLRE |
| 321 | ENLAIDKKGE LLTSEDLAHL KYTKKVVEET LRMANIGTFF |
| 361 | VRTAEKDVTY RGNKIPKNWL ILLWTRYLHN NTENFEDPMK |
| 401 | FNPDRWDETP KPGTFQPFGL GPRICPANML SKTQLVIFIH |
| 441 | HVVVGYKWEL TNPNVKISYV PQPMPSDGLE INFSKL |

A nucleotide sequence that encodes the Delphinium grandiflorum CYP729G1 enzyme of SEQ ID NO:11 is shown below as SEQ ID NO:12.

| | |
|---|---|
| 1 | ATGGAGCTCA CACAAGCACA GGCATGGGG TCTGCTCTTG |
| 41 | TCTTTACTAT CTTACCTTTT CTTGTGTGGC TCGTCTTCTC |
| 81 | ATGGAATGAG CTCAGATATG TGAAAACTCA GTCCAGTGAT |
| 121 | GGAGGCAAGC TTCCACCAGG GCATCTTGGT TTGCCAGTTA |
| 161 | TCGGCCAACT CCTCAGCTTC ATTTGGTATT TCAGAATTCG |
| 201 | CCGGAACCCC GATGATTTCG TCCATTCAAT GAGAAAAAGA |
| 241 | TACGGAGATG CTGATGGAAT ATATCGAAGC TACCTCTTTG |
| 281 | GATCTCCGGC AATCATCGGC TGCTCCCCAG ATTTCAACAA |
| 321 | GTTTGTCCTA CAATCAAGCA ATTTGTTTCA AGCTACCCGA |
| 361 | CGTCAAAAGG ATATTTTTGG CCATAATTCT GTTGCAGTAG |
| 401 | TTAATGGTAA AGCACATTAC AGACTTAGGG GTTACATCAA |
| 441 | CAATACAATC AGTACTCCTG ATGCTCTAAA GAAGATCACA |
| 481 | ATTTGTATAC AACCCAATAT AGTCTCCTCC CTCCAGTCAT |
| 521 | GGGCAGAGAA AGGTAAAATC AAAGGGGTAT ATGACATCAA |
| 561 | GAAGGTATTC TTTGAAACCA TCTGTATTAT AATCACTAGC |
| 601 | TTCAAACCTG GCCCCGCAAT AGATATGCTT GATCAACACT |
| 641 | TTCATGCCAT TCTTGACGGA CTTGGAGAAA AAGGGACAAA |
| 681 | GTTTCACCTA GCAGTTCAGA GTAAAAAGAC ATTGACTGAA |
| 721 | GTTTTCAAGA AAGAAATTGA TAAAAGAACG CAACATGGTA |
| 761 | TTCCATCAGA GGACCAAAAT GATCTGATGG AAAGATTGAT |
| 801 | GAGAATGAGA GATGAGGATG GAGAACCATT AAGTGATGAT |
| 841 | GAGGTGATTG ATAATATTGT GACTTGTATC ATGGGTGGCT |
| 881 | ATGAATCACC TTTCCAACTT GCGATATGGG CTCTTTACTT |
| 921 | TCTAGCCAAG AACAATGATG TGCTTCAAAA ACTCCGGGAA |
| 961 | GAAAATCTAG CCATAGATAA GAAAGGAGAA TTGTTAACAA |
| 1001 | GTGAAGATCT TGCACACTTG AAGTACACGA AGAAGGTGGT |
| 1041 | GGAAGAAACT CTAAGAATGG CAAACATTGG AACTTTCTTT |
| 1081 | GTTAGGACAG CAGAAAAGGA TGTTACTTAT CGAGGTAATA |
| 1121 | AAATACCAAA GAATTGGCTT ATACTTCTAT GGACGCGCTA |
| 1161 | TCTTCATAAT AATACAGAAA ATTTTGAAGA CCCCATGAAG |
| 1201 | TTCAATCCTG ATAGATGGGA TGAAACTCCA AAGCCCGGCA |
| 1241 | CATTTCAACC ATTTGGTTTG GGTCCAAGGA TTTGTCCAGC |
| 1281 | AAACATGCTT TCTAAAACTC AACTTGTTAT TTTTATTCAT |
| 1321 | CATGTGGTGG TCGGATACAA GTGGGAACTG ACAAATCCAA |
| 1361 | ATGTGAAAAT AAGCTATGTT CCACAACCAA TGCCATCAGA |
| 1401 | TGGATTGGAG ATTAATTTCA GTAAATTATA G |

An amino acid sequence for the Delphinium grandiflorum CYP71FK1 enzyme is shown below as SEQ ID NO:13.

| | |
|---|---|
| 1 | MENVVQQVAT SNNPFFLLFL SLVFLLLVLK FKFTINTINP |
| 41 | KFPPSPRKLP FIGNAHQLVG GALHHVLHSL SQKHGPLMFL |
| 81 | HLVSRPTLVV SDANTAREVM KTYDHIFSSR PQLGIPNRLL |
| 121 | YGKDVAFAPY GEYWRQVKKI CVTQLLSAKK VQSFRVVREE |
| 161 | EVALAMDQMD QIEAASSGIN LSELFAGILG SVVCRVALGR |
| 201 | KYDTQGGGGR KFKKIVTEMT NLLGVINIAD LVPSLGWLNH |
| 241 | FNGLNARVEK NERDIDSELD GVIEEHLAKK RGGEVEEEDI |
| 281 | VDIMLRNEED STLGIPITRE ATKGVVLDMF AAGIETSSIV |
| 321 | LQWAMSELMK HPEIMLEVQK EVRDVAKGKH ILTENDINEM |
| 361 | HQLKSVIKET MRLHPPFPLL ILRESVKDVN IEGYHVPAKT |
| 401 | TVIINAVAIG KDQMWWEEPE RFLPKRFMNG RSTMVDFKGQ |
| 441 | DFQLIPFGAG RRICPGMLFA TSITELTFAN LLNRFDWIMP |
| 481 | NGVASDELDM KEGSGITIHR KFDLVLIAKP YHEICVE |

A nucleotide sequence that encodes the Delphinium grandiflorum CYP71FK1 enzyme of SEQ ID NO:13 is shown below as SEQ ID NO:14.

| | |
|---|---|
| 1 | ATGGAGAATG TAGTACAGCA AGTAGCTACT TCAAATAATC |
| 41 | CCTTCTTCCT CCTCTTCCTC TCTCTTGTCT TTCTTCTTCT |
| 81 | AGTGCTCAAG TTTAAGTTTA CTACAAACAC AACTAACCCC |
| 121 | AAATTCCCTC CTTCCCCACG GAAGCTTCCC TTCATAGGAA |
| 161 | ACGCACACCA ACTCGTCGGG GGTGCTCTTC ACCATGTTCT |
| 201 | CCACTCGCTA TCCCAAAAGC ATGGCCCCTT GATGTTCTTG |
| 241 | CACCTTGTTT CCAGACCAAC CCTAGTTGTA TCGGATGCTA |
| 281 | ATACCGCCCG AGAAGTTATG AAGACTTACG ATCATATCTT |
| 321 | TTCAAGTAGG CCTCAACTTG GGATTCCTAA CCGACTGCTA |
| 361 | TACGGTAAGG ATGTTGCCTT TGCACCCTAC GGGGAGTACT |
| 401 | GGAGGCAAGT GAAGAAGATA TGCGTCACAC AGCTTTTAAG |
| 441 | TGCTAAGAAG GTCCAGTCGT TTCGGGTTGT TAGAGAAGAA |
| 481 | GAAGTAGCTC TTGCCATGGA TCAAATGGAT CAAATAGAGG |
| 521 | CTGCCTCTTC GGGGATTAAT TTGAGCGAAT TATTTGCTGG |
| 561 | TATTTTGGGT AGTGTAGTTT GTAGGGTTGC CTTGGGGAGA |
| 601 | AAGTATGATA CACAAGGAGG AGGTGGTAGG AAGTTTAAGA |
| 641 | AGATTGTAAC TGAAATGACA AATTTGTTGG GAGTTACAAA |
| 681 | TATAGCCGAC CTAGTACCCT CACTTGGTTG GTTAAATCAT |
| 721 | TTTAATGGGT TGAATGCGCG GGTTGAGAAG AATTTCCGCG |
| 761 | ACATTGATTC TTTCTTAGAT GGAGTAATTG AAGAACATTT |
| 801 | GGCCAAGAAG AGAGGTGGTG AAGTAGAAGA AGAAGATATA |
| 841 | GTAGACATTA TGCTCAGGAA TGAAGAAGAC TCTACTCTTG |
| 881 | GAATTCCCAT AACAAGAGAA GCCACTAAAG GAGTCGTACT |
| 921 | GGATATGTTT GCAGCTGGGA TCGAAACTTC GTCAATAGTT |
| 961 | TTACAGTGGG CAATGTCCGA GCTGATGAAA CATCCTGAAA |
| 1001 | TCATGTTAGA AGTACAAAAG GAGGTCAGAG ATGTTGCTAA |
| 1041 | AGGAAAGCAC ATATTAACTG AAAATGATAT AAACGAAATG |
| 1081 | CACCAATTGA AATCAGTTAT TAAAGAGACT ATGAGATTGC |
| 1121 | ATCCTCCATT TCCTTTGTTG ATTCTTCGTG AATCGGTAAA |
| 1161 | AGATGTAAAC ATTGAGGGCT ATCACGTTCC TGCAAAAACA |
| 1201 | ACTGTCATAA TCAATGCAGT TGCAATCGGT AAAGATCAAA |
| 1241 | TGTGGTGGGA AGAGCCTGAG AGATTTTTGC CAAAGAGATT |
| 1281 | TATGAACGGT AGGAGTACAA TGGTTGATTT TAAAGGACAA |
| 1321 | GATTTTCAAC TAATTCCATT TGGAGCGGGT AGGAGAATAT |
| 1361 | GCCCTGGAAT GCTTTTTGCA ACATCCATAA CTGAACTTAC |
| 1401 | TTTTGCGAAT CTTCTTAACA GATTTGATTG GATCATGCCA |
| 1441 | AATGGAGTGG CCAGTGATGA ATTAGATATG AAAGAAGGTT |
| 1481 | CTGGGATTAC AATTCATAGG AAATTTGATC TCGTTCTTAT |
| 1521 | TGCAAAGCCA TATCATGAAA TATGTGTTGA ATAA |

Variants in sequences can occur amongst members of a species. In many cases such sequence variants still retain good enzyme activity. Enzymes described herein can have one or more deletions, insertions, replacements, or substitutions in a part of the enzyme. The enzyme(s) described herein can have, for example, at least 60%, or at least 70%, or at least 80%, or at least 90%, or at least 93%, or at least 95%, or at least 96%, or at least 97%, or at least 98%, or at least 99% sequence identity to a sequence described herein.

In some cases, enzymes can have conservative changes such as one or more deletions, insertions, replacements, or substitutions that have no significant effect on the activities of the enzymes. Examples of conservative substitutions are provided below in Table 1A.

TABLE 1A
Conservative Substitutions

| Type of | |
|---|---|
| Amino Acid | Substitutable Amino Acids |
| Hydrophilic | Ala, Pro, Gly, Glu, Asp, Gln, Asn, Ser, Thr |
| Sulfhydryl | Cys |
| Aliphatic | Val, Ile, Leu, Met |
| Basic | Lys, Arg, His |
| Aromatic | Phe, Tyr, Trp |

Nucleic acids encoding the enzymes can have also have sequence variations. For example, nucleic acid sequences described herein can be modified to express enzymes that do not have modifications. Most amino acids can be encoded by more than one codon. When an amino acid is encoded by more than one codon, the codons are referred to as degenerate codons. A listing of degenerate codons is provided in Table 1B below.

TABLE 1B
Degenerate Amino Acid Codons

| Amino | |
|---|---|
| Acid | Three Nucleotide Codon |
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |
| START | ATG |
| STOP | TAG, TGA, TAA |

Different organisms may translate different codons more or less efficiently (e.g., because they have different ratios of tRNAs) than other organisms. Hence, when some amino acids can be encoded by several codons, a nucleic acid segment can be designed to optimize the efficiency of expression of an enzyme by using codons that are preferred by an organism of interest. For example, the nucleotide coding regions of the enzymes described herein can be codon optimized for expression in various plant species.

An optimized nucleic acid can have less than 98%, less than 97%, less than 96%, less than 95%, or less than 94%, or less than 93%, or less than 92%, or less than 91%, or less than 90%, or less than 89%, or less than 88%, or less than 85%, or less than 83%, or less than 80%, or less than 75% nucleic acid sequence identity to a corresponding non-optimized (e.g., a non-optimized parental or wild type enzyme nucleic acid) sequence.

The enzymes described herein can be expressed from an expression cassette and/or an expression vector. Such an expression cassette can include a nucleic acid segment that encodes an enzyme operably linked to a promoter to drive expression of the enzyme. Convenient vectors, or expression systems can be used to express such enzymes. In some instances, the nucleic acid segment encoding an enzyme is operably linked to a promoter and/or a transcription termination sequence. The promoter and/or the termination sequence can be heterologous to the nucleic acid segment that encodes an enzyme. Expression cassettes can have a promoter operably linked to a heterologous open reading frame encoding an enzyme. The invention therefore provides expression cassettes or vectors useful for expressing one or more enzyme(s).

Constructs, e.g., expression cassettes, and vectors comprising the isolated nucleic acid molecule, e.g., with optimized nucleic acid sequence, as well as kits comprising the isolated nucleic acid molecule, construct or vector are also provided.

The nucleic acids described herein can also be modified to improve or alter the functional properties of the encoded enzymes. Deletions, insertions, or substitutions can be generated by a variety of methods such as, but not limited to, random mutagenesis and/or site-specific recombination-mediated methods. The mutations can range in size from one or two nucleotides to hundreds of nucleotides (or any value there between). Deletions, insertions, and/or substitutions are created at a desired location in a nucleic acid encoding the enzyme(s).

Nucleic acids encoding one or more enzyme(s) can have one or more nucleotide deletions, insertions, replacements, or substitutions. For example, the nucleic acids encoding one or more enzyme(s) can, for example, have less than 95%, or less than 94.8%, or less than 94.5%, or less than 94%, or less than 93.8%, or less than 94.50% nucleic acid sequence identity to a corresponding parental or wild-type sequence. In some cases, the nucleic acids encoding one or more enzyme(s) can have, for example, at least 50%, or at least 55%, or at least 60%, or at least 65%, or at least 70%, or at least 75%, or at least 80%, or at least 85%, or at 90% sequence identity to a corresponding parental or wild-type sequence. Examples of parental or wild type nucleic acid sequences for unmodified enzyme(s) with amino acid sequences SEQ ID NOs:1, 3, 5, 7, 9, 11, or 13, include nucleic acid sequences SEQ ID NOs:2, 4, 6, 8, 10, 12, or 14, respectively. Any of these nucleic acid or amino acid sequences can, for example, encode or have enzyme sequences with less than 100%, less than 99%, less than 98%, less than 97%, less than 96%, less than 95%, less than 94.8%, less than 94.5%, less than 94%, less than 93.8%, less than 93.5%, less than 93%, less than 92%, less than 91%, or less than 90% sequence identity to a corresponding parental or wild-type sequence.

Also provided are nucleic acid molecules (polynucleotide molecules) that can include a nucleic acid segment encoding an enzyme with a sequence that is optimized for expression in at least one selected host organism or host cell. Optimized sequences include sequences which are codon optimized, i.e., codons which are employed more frequently in one organism relative to another organism. In some cases, the balance of codon usage is such that the most frequently used codon is not used to exhaustion. Other modifications can include addition or modification of Kozak sequences and/or introns, and/or to remove undesirable sequences, for instance, potential transcription factor binding sites.

An enzyme useful for synthesis of terpenes, diterpenes, diterpenoid alkaloids, and terpenoids may be expressed on the surface of, or within, a prokaryotic or eukaryotic cell. In some cases, expressed enzyme(s) can be secreted by that cell.

Techniques of molecular biology, microbiology, and recombinant DNA technology which are within the skill of the art can be employed to make and use the enzymes, expression systems, and terpene products described herein. Such techniques available in the literature. See, e.g., Sambrook, Fritsch & Maniatis, Molecular Cloning: A Laboratory Manual, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series Methods In Enzymology (S. Colowick and N. Kaplan eds., Academic Press, Inc.); Current Protocols In Molecular Biology (John Wiley & Sons, Inc), Current Protocols In Protein Science (John Wiley & Sons, Inc), Current Protocols In Microbiology (John Wiley & Sons, Inc), Current Protocols In Nucleic Acid Chemistry (John Wiley & Sons, Inc), and Handbook of Experimental Immunology, Vols. I-IV (D. M. Weir and C. C. Blackwell eds., 1986, Blackwell Scientific Publications).

Modified plants that contain nucleic acids encoding enzymes within their somatic and/or germ cells are described herein. Such genetic modification can be accomplished by available procedures. For example, one of skill in the art can prepare an expression cassette or expression vector that can express one or more encoded enzymes. Plant cells can be transformed by the expression cassette or expression vector, and whole plants (and their seeds) can be generated from the plant cells that were successfully transformed with the enzyme nucleic acids. Some procedures for making such genetically modified plants and their seeds are described below.

Promoters: The nucleic acids encoding enzymes can be operably linked to a promoter, which provides for expression of mRNA from the nucleic acids encoding the enzymes. The promoter is typically a promoter functional in plants and can be a promoter functional during plant growth and development. A nucleic acid segment encoding an enzyme is operably linked to the promoter when it is located downstream from the promoter. The combination of a coding region for an enzyme operably linked to a promoter forms an expression cassette, which can optionally include other elements as well.

Promoter regions are typically found in the flanking DNA upstream from the coding sequence in both the prokaryotic and eukaryotic cells. A promoter sequence provides for regulation of transcription of the downstream gene sequence and typically includes from about 50 to about 2,000 nucleotide base pairs. Promoter sequences also contain regulatory sequences such as enhancer sequences that can influence the level of gene expression. Some isolated promoter sequences can provide for gene expression of heterologous DNAs, that is a DNA different from the native or homologous DNA.

Promoter sequences are also known to be strong or weak, or inducible. A strong promoter provides for a high level of gene expression, whereas a weak promoter provides for a very low level of gene expression. An inducible promoter is a promoter that provides for the turning gene expression on and off in response to an exogenously added agent, or to an environmental or developmental stimulus. For example, a bacterial promoter such as the P_{tac} promoter can be induced to varying levels of gene expression depending on the level of isopropyl-beta-D-thiogaiactoside added to the transformed cells. Promoters can also provide for tissue specific or developmental regulation. An isolated promoter sequence that is a strong promoter for heterologous DNAs is advantageous because it provides for a sufficient level of gene expression for easy detection and selection of transformed cells and provides for a high level of gene expression when desired.

Expression cassettes generally include, but are not limited to, examples of plant promoters such as the CaMV 35S promoter (Odell et al., Nature. 313:810-812 (1985)), or others such as CaMV 19S (Lawton et al., Plant Molecular Biology. 9:315-324 (1987)), nos (Ebert et al., Proc. Natl. Acad. Sci. USA. 84:5745-5749 (1987)), Adh1 (Walker et al., Proc. Natl. Acad. Sci. USA. 84:6624-6628 (1987)), sucrose synthase (Yang et al., Proc. Natl. Acad. Sci. USA. 87:4144-4148 (1990)), α-tubulin, ubiquitin, actin (Wang et al., Mol. Cell. Biol. 12:3399 (1992)), cab (Sullivan et al., Mol. Gen. Genet. 215:431 (1989)), PEPCase (Hudspeth et al., Plant Molecular Biology. 12:579-589 (1989)) or those associated with the R gene complex (Chandler et al., The Plant Cell. 1:1175-1183 (1989)). Further suitable promoters include a CYP71D16 trichome-specific promoter and the CBTS (cembratrienol synthase) promotor, cauliflower mosaic virus promoter, the Z10 promoter from a gene encoding a 10 kD zein protein, a Z27 promoter from a gene encoding a 27 kD zein protein, the plastid rRNA-operon (rrn) promoter, inducible promoters, such as the light inducible promoter derived from the pea rbcS gene (Coruzzi et al., EMBO J. 3:1671 (1971)), RUBISCO-SSU light inducible promoter (SSU) from tobacco and the actin promoter from rice (McElroy et al., The Plant Cell. 2:163-171 (1990)). Other promoters that are useful can also be employed.

Alternatively, novel tissue specific promoter sequences may be employed. cDNA clones from a particular tissue can be isolated and those clones which are expressed specifically in that tissue can be identified, for example, using Northern blotting. Preferably, the gene isolated is not present in a high copy number but is relatively abundant in specific tissues. The promoter and control elements of corresponding genomic clones can then be localized using techniques well known to those of skill in the art.

A nucleic acid encoding an enzyme can be combined with the promoter by standard methods to yield an expression cassette, for example, as described in Sambrook et al. (MOLECULAR CLONING: A LABORATORY MANUAL. Second Edition (Cold Spring Harbor, NY: Cold Spring Harbor Press (1989); MOLECULAR CLONING: A LABORATORY MANUAL. Third Edition (Cold Spring Harbor, NY: Cold Spring Harbor Press (2000)). Briefly, a plasmid containing a promoter such as the 35S CaMV promoter or the CYP71D16 trichome-specific promoter can be constructed as described in Jefferson (Plant Molecular Biology Reporter 5:387-405 (1987)) or obtained from Clontech Lab in Palo Alto, California (e.g., pBI121 or pBI221). Typically, these plasmids are constructed to have multiple cloning sites having specificity for different restriction enzymes downstream from the promoter.

The nucleic acid sequence encoding for the enzyme(s) can be subcloned downstream from the promoter using restriction enzymes and positioned to ensure that the DNA is inserted in proper orientation with respect to the promoter so that the DNA can be expressed as sense RNA. Once the nucleic acid segment encoding the enzyme is operably linked to a promoter, the expression cassette so formed can be subcloned into a plasmid or other vector (e.g., an expression vector).

In some embodiments, a cDNA clone encoding an enzyme is isolated from Delphinium grandiflorum, for example, from leaf, trichome, or root tissue. In other embodiments, cDNA clones from other species (that encode an enzyme) are isolated from selected plant tissues, or a nucleic acid encoding a wild type, mutant or modified enzyme is prepared by available methods or as described herein. For example, the nucleic acid encoding the enzyme can be any nucleic acid with a coding region that hybridizes to SEQ ID NOs: 2, 4, 6, 8, 10, 12, or 14 and that has enzyme activity. Using restriction endonucleases, the entire coding sequence for the enzyme is subcloned downstream of the promoter in a 5′ to 3′ sense orientation.

Targeting Sequences: Additionally, expression cassettes can be constructed and employed to target the nucleic acids encoding an enzyme to an intracellular compartment within plant cells or to direct an encoded protein to the extracellular environment. This can generally be achieved by joining a DNA sequence encoding a transit or signal peptide sequence to the coding sequence of the nucleic acid encoding the enzyme. The resultant transit, or signal, peptide can transport the protein to a particular intracellular, or extracellular, destination and can then be co-translationally or post-translationally removed. Transit peptides act by facilitating the transport of proteins through intracellular membranes, e.g., vacuole, vesicle, plastid and mitochondrial membranes, whereas signal peptides direct proteins through the extracellular membrane. By facilitating transport of the protein into compartments inside or outside the cell, these sequences can increase the accumulation of a particular gene product within a particular location. For example, see U.S. Pat. No. 5,258,300.

For example, in some cases it may be desirable to localize the enzymes to the plastidic compartment and/or within plant cell trichomes. The best compliment of transit peptides/secretion peptide/signal peptides can be empirically ascertained. The choices can range from using the native secretion signals akin to the enzyme candidates to be transgenically expressed, to transit peptides from proteins known to be localized into plant organelles such as trichome plastids in general. For example, transit peptides can be selected from proteins that have a relative high titer in the trichomes. Examples include, but not limited to, transit peptides form a terpenoid cyclase (e.g. cembratrieneol cyclase), the LTP1 protein, the Chlorophyll a-b binding protein 40, Phylloplanin, Glycine-rich Protein (GRP), Cytochrome P450 (CYP71D16); all from Nicotiana sp. alongside RUBISCO (Ribulose bisphosphate carboxylase) small unit protein from both Arabidopsis and Nicotiana sp.

3′ Sequences: When the expression cassette is to be introduced into a plant cell, the expression cassette can also optionally include 3′ untranslated plant regulatory DNA sequences that act as a signal to terminate transcription and allow for the polyadenylation of the resultant mRNA. The 3′ untranslated regulatory DNA sequence can include from about 300 to 1,000 nucleotide base pairs and can contain plant transcriptional and translational termination sequences. For example, 3′ elements that can be used include those derived from the nopaline synthase gene of Agrobacterium tumefaciens (Bevan et al., Nucleic Acid Research. 11:369-385 (1983)), or the terminator sequences for the T7 transcript from the octopine synthase gene of Agrobacterium tumefaciens, and/or the 3′ end of the protease inhibitor I or II genes from potato or tomato. Other 3′ elements known to those of skill in the art can also be employed. These 3′ untranslated regulatory sequences can be obtained as described in An (Methods in Enzymology. 153:292 (1987)). Many such 3′ untranslated regulatory sequences are already present in plasmids available from commercial sources such as Clontech, Palo Alto, California. The 3′ untranslated regulatory sequences can be operably linked to the 3′ terminus of the nucleic acids encoding the enzyme.

Selectable and Screenable Marker Sequences: To improve identification of transformants, a selectable or screenable marker gene can be employed with the expressible nucleic acids encoding the enzyme(s). “Marker genes” are genes that impart a distinct phenotype to cells expressing the marker gene and thus allow such transformed cells to be distinguished from cells that do not have the marker. Such genes may encode either a selectable or a screenable marker, depending on whether the marker confers a trait which one can ‘select’ for by chemical means, i.e., through the use of a selective agent (e.g., a herbicide, antibiotic, or the like), or whether it is simply a trait that one can identify through observation or testing, i.e., by ‘screening’ (e.g., the R-locus trait). Of course, many examples of suitable marker genes are available can be employed in the practice of the invention.

Included within the terms ‘selectable or screenable marker genes’ are also genes which encode a “secretable marker” whose secretion can be detected as a means of identifying or selecting for transformed cells. Examples include markers which encode a secretable antigen that can be identified by antibody interaction, or secretable enzymes that can be detected by their catalytic activity. Secretable proteins fall into a number of classes, including small, diffusible proteins detectable, e.g., by ELISA; and proteins that are inserted or trapped in the cell wall (e.g., proteins that include a leader sequence such as that found in the expression unit of extensin or tobacco PR-S).

With regard to selectable secretable markers, the use of an expression system that encodes a polypeptide that becomes sequestered in the cell wall, where the polypeptide includes a unique epitope may be advantageous. Such a cell wall antigen can employ an epitope sequence that would provide low background in plant tissue, a promoter-leader sequence that imparts efficient expression and targeting across the plasma membrane, and that can produce protein that is bound in the cell wall and yet is accessible to antibodies. A normally secreted cell wall protein modified to include a unique epitope would satisfy such requirements.

Example of protein markers suitable for modification in this manner include extensin or hydroxyproline rich glycoprotein (HPRG). For example, the maize HPRG (Stiefel et al., The Plant Cell. 2:785-793 (1990)) is well characterized in terms of molecular biology, expression, and protein structure and therefore can readily be employed. However, any one of a variety of extensins and/or glycine-rich cell wall proteins (Keller et al., EMBO J. 8:1309-1314 (1989)) could be modified by the addition of an antigenic site to create a screenable marker.

Selectable markers for use in connection with the present invention can include, but are not limited to, a neo gene (Potrykus et al., Mol. Gen. Genet. 199:183-188 (1985)) which codes for kanamycin resistance and can be selected for using kanamycin, G418; a bar gene which codes for bialaphos resistance; a gene which encodes an altered EPSP synthase protein (Hinchee et al., Bio/Technology. 6:915-922 (1988)) thus conferring glyphosate resistance; a nitrilase gene such as bxn from Klebsiella ozaenae which confers resistance to bromoxynil (Stalker et al., Science. 242:419-423 (1988)); a mutant acetolactate synthase gene (ALS) which confers resistance to imidazolinone, sulfonylurea or other ALS-inhibiting chemicals (European Patent Application 154,204 (1985)); a methotrexate-resistant DHFR gene (Thillet et al., J. Biol. Chem. 263:12500-12508 (1988)); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (European Patent Application 0 218 571 (1987)).

An illustrative embodiment of a selectable marker gene capable of being used in systems to select transformants is the gene that encode the enzyme phosphinothricin acetyltransferase, such as the bar gene from Streptomyces hygroscopicus or the pat gene from Streptomyces viridochromogenes (U.S. Pat. No. 5,550,318). The enzyme phosphinothricin acetyl transferase (PAT) inactivates the active ingredient in the herbicide bialaphos, phosphinothricin (PPT). PPT inhibits glutamine synthetase, (Murakami et al., Mol. Gen. Genet. 205:42-50 (1986); Twell et al., Plant Physiol. 91:1270-1274 (1989)) causing rapid accumulation of ammonia and cell death. Screenable markers that may be employed include, but are not limited to, a β-glucuronidase or uidA gene (GUS) that encodes an enzyme for which various chromogenic substrates are known; an R-locus gene, which encodes a product that regulates the production of anthocyanin pigments (red color) in plant tissues (Dellaporta et al., In: Chromosome Structure and Function: Impact of New Concepts, 18^{th} Stadler Genetics Symposium, J. P. Gustafson and R. Appels, eds. (New York: Plenum Press) pp. 263-282 (1988)); a β-lactamase gene (Sutcliffe, Proc. Natl. Acad. Sci. USA. 75:3737-3741(1978)), which encodes an enzyme for which various chromogenic substrates are known (e.g., PADAC, a chromogenic cephalosporin); a xylE gene (Zukowsky et al., Proc. Natl. Acad. Sci. USA. 80:1101 (1983)) which encodes a catechol dioxygenase that can convert chromogenic catechols; an α-amylase gene (Ikuta et al., Bio/technology 8:241-242 (1990)); a tyrosinase gene (Katz et al., J. Gen. Microbiol. 129:2703-2714 (1983)) which encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., Science. 234:856-859.1986), which allows for bioluminescence detection; or an aequorin gene (Prasher et al., Biochem. Biophys. Res. Comm. 126:1259-1268 (1985)), which may be employed in calcium-sensitive bioluminescence detection, or a green or yellow fluorescent protein gene (Niedz et al., Plant Cell Reports. 14:403 (1995)).

Another screenable marker contemplated for use is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for population screening for bioluminescence, such as on tissue culture plates, or even for whole plant screening.

Other Optional Sequences: An expression cassette of the invention can also include plasmid DNA. Plasmid vectors include additional DNA sequences that provide for easy selection, amplification, and transformation of the expression cassette in prokaryotic and eukaryotic cells, e.g., pUC-derived vectors such as pUC8, pUC9, pUC18, pUC19, pUC23, pUC119, and pUC120, pSK-derived vectors, pGEM-derived vectors, pSP-derived vectors, or pBS-derived vectors. The additional DNA sequences can include origins of replication to provide for autonomous replication of the vector, additional selectable marker genes, for example, encoding antibiotic or herbicide resistance, unique multiple cloning sites providing for multiple sites to insert DNA sequences or genes encoded in the expression cassette and sequences that enhance transformation of prokaryotic and eukaryotic cells.

Another vector that is useful for expression in both plant and prokaryotic cells is the binary Ti plasmid (as disclosed in Schilperoort et al., U.S. Pat. No. 4,940,838) as exemplified by vector pGA582. This binary Ti plasmid vector has been previously characterized by An (Methods in Enzymology. 153:292 (1987)) and is available from Dr. An. This binary Ti vector can be replicated in prokaryotic bacteria such as E. coli and Agrobacterium. The Agrobacterium plasmid vectors can be used to transfer the expression cassette to dicot plant cells, and under certain conditions to monocot cells, such as rice cells. The binary Ti vectors can include the nopaline T DNA right and left borders to provide for efficient plant cell transformation, a selectable marker gene, unique multiple cloning sites in the T border regions, the colE1 replication of origin and a wide host range replicon. The binary Ti vectors carrying an expression cassette of the invention can be used to transform both prokaryotic and eukaryotic cells but is usually used to transform dicot plant cells.

DNA Delivery of the DNA Molecules into Host Cells: Methods described herein can include introducing nucleic acids encoding enzymes, such as a preselected cDNA encoding the selected enzyme, into a recipient cell to create a transformed cell. In some instances, the frequency of occurrence of cells taking up exogenous (foreign) DNA may be low. Moreover, it is most likely that not all recipient cells receiving DNA segments or sequences will result in a transformed cell wherein the DNA is stably integrated into the plant genome and/or expressed. Some recipient cells may show only initial and transient gene expression. However, certain cells from virtually any dicot or monocot species may be stably transformed, and these cells regenerated into transgenic plants, through the application of the techniques disclosed herein.

Another aspect of the invention is a plant that can produce terpenes, diterpenes, diterpenoid alkaloids, and terpenoids, wherein the plant has introduced nucleic acid sequence(s) encoding one or more enzymes. The plant can be a monocotyledon or a dicotyledon. Another aspect of the invention includes plant cells (e.g., embryonic cells or other cell lines) that can regenerate fertile transgenic plants and/or seeds. The cells can be derived from either monocotyledons or dicotyledons. In some embodiments, the plant or cell is a monocotyledon plant or cell. In some embodiments, the plant or cell is a dicotyledon plant or cell. For example, the plant or cell can be a tobacco plant or cell. The cell(s) may be in a suspension cell culture or may be in an intact plant part, such as an immature embryo, or in a specialized plant tissue, such as callus, such as Type I or Type II callus.

Transformation of plant cells can be conducted by any one of a number of methods available in the art. Examples are: Transformation by direct DNA transfer into plant cells by electroporation (U.S. Pat. Nos. 5,384,253 and 5,472,869, Dekeyser et al., The Plant Cell. 2:591-602 (1990)); direct DNA transfer to plant cells by PEG precipitation (Hayashimoto et al., Plant Physiol. 93:857-863 (1990)); direct DNA transfer to plant cells by microprojectile bombardment (McCabe et al., Bio/Technology. 6:923-926 (1988); Gordon-Kamm et al., The Plant Cell. 2:603-618 (1990); U.S. Pat. Nos. 5,489,520; 5,538,877; and 5,538,880) and DNA transfer to plant cells via infection with Agrobacterium. Methods such as microprojectile bombardment or electroporation can be carried out with “naked” DNA where the expression cassette may be simply carried on any E. coli-derived plasmid cloning vector. In the case of viral vectors, it is desirable that the system retain replication functions, but lack the functions for disease induction.

One method for dicot transformation, for example, involves infection of plant cells with Agrobacterium tumefaciens using the leaf-disk protocol (Horsch et al., Science 227:1229-1231 (1985). Methods for transformation of monocotyledonous plants utilizing Agrobacterium tumefaciens have been described by Hiei et al. (European Patent 0 604 662, 1994) and Saito et al. (European Patent 0 672 752, 1995).

Monocot cells such as various grasses or dicot cells such as tobacco can be transformed via microprojectile bombardment of embryogenic callus tissue or immature embryos, or by electroporation following partial enzymatic degradation of the cell wall with a pectinase-containing enzyme (U.S. Pat. Nos. 5,384,253; and 5,472,869). For example, embryogenic cell lines derived from immature embryos can be transformed by accelerated particle treatment as described by Gordon-Kamm et al. (The Plant Cell. 2:603-618 (1990)) or U.S. Pat. Nos. 5,489,520; 5,538,877 and U.S. Pat. No. 5,538,880, cited above. Excised immature embryos can also be used as the target for transformation prior to tissue culture induction, selection and regeneration as described in U.S. application Ser. No. 08/112,245 and PCT publication WO 95/06128.

The choice of plant tissue source for transformation may depend on the nature of the host plant and the transformation protocol. Useful tissue sources include callus, suspensions culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells.

The transformation is carried out under conditions directed to the plant tissue of choice. The plant cells or tissue are exposed to the DNA or RNA encoding enzymes for an effective period of time. This may range from a less than one second pulse of electricity for electroporation to a 2-day to 3-day co-cultivation in the presence of plasmid-bearing Agrobacterium cells. Buffers and media used will also vary with the plant tissue source and transformation protocol. Many transformation protocols employ a feeder layer of suspended culture cells (tobacco, for example) on the surface of solid media plates, separated by a sterile filter paper disk from the plant cells or tissues being transformed.

Electroporation: Where one wishes to introduce DNA by means of electroporation, it is contemplated that the method of Krzyzek et al. (U.S. Pat. No. 5,384,253) may be advantageous. In this method, certain cell wall-degrading enzymes, such as pectin-degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin-degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

Microprojectile Bombardment: A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. In an illustrative embodiment, non-embryogenic BMS cells were bombarded with intact cells of the bacteria E. coli or Agrobacterium tumefaciens containing plasmids with either the β-glucoronidase or bar gene engineered for expression in selected plant cells. Bacteria were inactivated by ethanol dehydration prior to bombardment. A low level of transient expression of the β-glucoronidase gene was observed 24-48 hours following DNA delivery. In addition, stable transformants containing the bar gene were recovered following bombardment with either E. coli or Agrobacterium tumefaciens cells. It is contemplated that particles may contain DNA rather than be coated with DNA. Hence it is proposed that particles may increase the level of DNA delivery but are not, in and of themselves, necessary to introduce DNA into plant cells.

An advantage of microprojectile bombardment, in addition to being an effective means of reproducibly stably transforming monocots, microprojectile bombardment does not require the isolation of protoplasts (Christou et al., PNAS 84:3962-3966 (1987)), the formation of partially degraded cells, and no susceptibility to Agrobacterium infection is required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with maize cells cultured in suspension (Gordon-Kamm et al., The Plant Cell. 2:603-618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing the damage inflicted on recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein, one may obtain up to 1000 or more foci of cells transiently expressing a marker gene. The number of cells in a focus which express the exogenous gene product 48 hours post-bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macro- or microprojectiles. Biological factors include all steps involved in manipulation of cells before and immediately after bombardment, the osmotic adjustment of target cells to help alleviate the trauma associated with the bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore, influence transformation and integration efficiencies. For example, the osmotic state, tissue hydration and the subculture stage or cell cycle of the recipient cells may be adjusted for optimum transformation. Execution of such routine adjustments will be known to those of skill in the art.

Selection: An exemplary embodiment of methods for identifying transformed cells involves exposing the bombarded cultures to a selective agent, such as a metabolic inhibitor, an antibiotic, or the like. Cells which have been transformed and have stably integrated a marker gene conferring resistance to the selective agent used, will grow and divide in culture. Sensitive cells will not be amenable to further culturing.

To use the bar-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for identification of transformed cells. For example, selection with a growth inhibiting compound, such as bialaphos or glyphosate at concentrations that provide 100% inhibition followed by screening of growing tissue for expression of a screenable marker gene such as luciferase would allow one to recover transformants from cell or tissue types that are not amenable to selection alone.

Regeneration and Seed Production: Cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA+2,4-D or perhaps even picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO₂, and at about 25-250 microeinsteins/sec·m² of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to express the trait. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

Regenerated plants can be repeatedly crossed to inbred plants to introgress the nucleic acids encoding an enzyme into the genome of the inbred plants. This process is referred to as backcross conversion. When a sufficient number of crosses to the recurrent inbred parent have been completed in order to produce a product of the backcross conversion process that is substantially isogenic with the recurrent inbred parent except for the presence of the introduced nucleic acids, the plant is self-pollinated at least once in order to produce a homozygous backcross converted inbred containing the nucleic acids encoding the enzyme(s). Progeny of these plants are true breeding.

Alternatively, seed from transformed plants regenerated from transformed tissue cultures is grown in the field and self-pollinated to generate true breeding plants.

Seed from the fertile transgenic plants can then be evaluated for the presence and/or expression of the enzyme(s). Transgenic plant and/or seed tissue can be analyzed for enzyme expression using methods such as SDS polyacrylamide gel electrophoresis, Western blot, liquid chromatography (e.g., HPLC) or other means of detecting an enzyme product (e.g., a terpene, diterpene, terpenoid, diterpenoid alkaloid, or a combination thereof).

Once a transgenic seed expressing the enzyme(s) and producing one or more terpenes, diterpenes, diterpenoid alkaloids, and/or terpenoids in the plant is identified, the seed can be used to develop true breeding plants. The true breeding plants are used to develop a line of plants expressing terpenes, diterpenes, diterpenoid alkaloids, and/or terpenoids in various plant tissues (e.g., in leaves, bracts, and/or trichomes) while still maintaining other desirable functional agronomic traits. Adding the trait of terpene, diterpene, diterpenoid alkaloid, and/or terpenoid production can be accomplished by back-crossing with selected desirable functional agronomic trait(s) and with plants that do not exhibit such traits and studying the pattern of inheritance in segregating generations. Those plants expressing the target trait(s) in a dominant fashion are preferably selected. Back-crossing is carried out by crossing the original fertile transgenic plants with a plant from an inbred line exhibiting desirable functional agronomic characteristics while not necessarily expressing the trait of terpene, diterpene, diterpenoid alkaloid, and/or terpenoid production in the plant. The resulting progeny can then be crossed back to the parent that expresses the terpenes, diterpenes, diterpenoid alkaloids, and/or terpenoids. The progeny from this cross will also segregate so that some of the progeny carry the trait and some do not. This back-crossing is repeated until the goal of acquiring an inbred line with the desirable functional agronomic traits, and with production of terpenes, diterpenes, diterpenoid alkaloids, and/or terpenoids within various tissues of the plant is achieved. The enzymes can be expressed in a dominant fashion.

Subsequent to back-crossing, the new transgenic plants can be evaluated for synthesis of terpenes, diterpenes, diterpenoid alkaloids, and/or terpenoids in selected plant lines. This can be done, for example, by gas chromatography, mass spectroscopy, or NMR analysis of whole plant cell walls (Kim, H., and Ralph, J. Solution-state 2D NMR of ball-milled plant cell wall gels in DMSO-d₆/pyridine-ds. (2010) Org. Biomol. Chem. 8(3), 576-591; Yelle, D. J., Ralph, J., and Frihart, C. R. Characterization of non-derivatized plant cell walls using high-resolution solution-state NMR spectroscopy. (2008) Magn. Reson. Chem. 46(6), 508-517; Kim, H., Ralph, J., and Akiyama, T. Solution-state 2D NMR of Ball-milled Plant Cell Wall Gels in DMSO-d₆. (2008) BioEnergy Research 1(1), 56-66; Lu, F., and Ralph, J. Non-degradative dissolution and acetylation of ball-milled plant cell walls; high-resolution solution-state NMR. (2003) Plant J. 35(4), 535-544). The new transgenic plants can also be evaluated for a battery of functional agronomic characteristics such as lodging, yield, resistance to disease, resistance to insect pests, drought resistance, and/or herbicide resistance.

Determination of Stably Transformed Plant Tissues: To confirm the presence of the nucleic acids encoding terpene synthesizing enzymes in the regenerating plants, or seeds or progeny derived from the regenerated plant, a variety of assays may be performed. Such assays include, for example, molecular biological assays, such as Southern and Northern blotting and PCR; biochemical assays, such as detecting the presence of enzyme products, for example, by enzyme assays, by immunological assays (ELISAs and Western blots). Various plant parts can be assayed, such as trichomes, leaves, bracts, seeds or roots. In some cases, the phenotype of the whole regenerated plant can be analyzed.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from introduced nucleic acids. PCR can also be used to reverse transcribe RNA into DNA, using enzymes such as reverse transcriptase, and then this DNA can be amplified through the use of conventional PCR techniques. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and also demonstrate the presence or absence of an RNA species.

While Southern blotting may be used to detect the nucleic acid encoding the enzyme(s) in question, it may not provide information as to whether the preselected DNA segment is being expressed. Expression may be evaluated by specifically identifying the protein products of the introduced nucleic acids or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as, native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the enzyme such as evaluation by amino acid sequencing following purification. Other procedures may be additionally used.

The expression of a gene product can also be determined by evaluating the phenotypic results of its expression. These assays also may take many forms including but not limited to analyzing changes in the chemical composition, morphology, or physiological properties of the plant. Chemical composition may be altered by expression of preselected DNA segments encoding storage proteins which change amino acid composition and may be detected by amino acid analysis.

### Hosts

Terpenes, including diterpenes, diterpenoid alkaloids, and terpenoids, can be made in a variety of host organisms either in vitro or in vivo. In some cases, the enzymes described herein can be made in host cells, and those enzymes can be extracted from the host cells for use in vitro. As used herein, a “host” means a cell, tissue or organism capable of replication. The host can have an expression cassette or expression vector that can include a nucleic acid segment encoding an enzyme that is involved in the biosynthesis of terpenes.

The term “host cell”, as used herein, refers to any prokaryotic or eukaryotic cell that can be transformed with an expression cassettes or vector carrying the nucleic acid segment encoding an enzyme that is involved in the biosynthesis of one or more terpenes. The host cells can, for example, be a plant, bacterial, insect, or yeast cell. Expression cassettes encoding biosynthetic enzymes can be incorporated or transferred into a host cell to facilitate manufacture of the enzymes described herein or the terpene, diterpene, diterpenoid alkaloid, or terpenoid products of those enzymes. The host cells can be present in an organism. For example, the host cells can be present in a host such as a plant.

For example, the enzymes, terpenes, diterpenes, diterpenoid alkaloids, and terpenoids can be made in a variety of plants or plant cells. Although some of the enzymes described herein are from species of the mint family, the enzymes, terpenes, diterpenes, diterpenoid alkaloids, and terpenoids can be made in species other than in mint plants or mint plant cells. The terpenes, diterpenes, diterpenoid alkaloids, and terpenoids can, for example, be made and extracted from whole plants, plant parts, plant cells, or a combination thereof. Enzymes can conveniently, for example, be produced in bacterial, insect, plant, or fungal (e.g., yeast) cells.

Examples of host cells, host tissues, host seeds and plants that may be used for producing terpenes and terpenoids (e.g., by incorporation of nucleic acids and expression systems described herein) include but are not limited to those useful for production of oils such as oilseeds, camelina, canola, castor bean, corn, flax, lupins, peanut, potatoes, safflower, soybean, sunflower, cottonseed, oil firewood trees, rapeseed, rutabaga, sorghum, walnut, and various nut species. Other types host cells, host tissues, host seeds and plants that can be used include fiber-containing plants, trees, flax, grains (maize, wheat, barley, oats, rice, sorghum, millet and rye), grasses (switchgrass, prairie grass, wheat grass, sudangrass, sorghum, straw-producing plants), softwood, hardwood and other woody plants (e.g., poplar, pine, and eucalyptus), oil (oilseeds, camelina, canola, castor bean, lupins, potatoes, soybean, sunflower, cottonseed, oil firewood trees, rapeseed, rutabaga, sorghum), starch plants (wheat, potatoes, lupins, sunflower and cottonseed), and forage plants (alfalfa, clover and fescue). In some embodiments the plant is a gymnosperm.

Examples of plants useful for pulp and paper production include most pine species such as loblolly pine, Jack pine, Southern pine, Radiata pine, spruce, Douglas fir and others. Hardwoods that can be modified as described herein include aspen, poplar, eucalyptus, and others. Plants useful for making biofuels and ethanol include corn, grasses (e.g., miscanthus, switchgrass, and the like), as well as trees such as poplar, aspen, pine, oak, maple, walnut, rubber tree, willow, and the like. Plants useful for generating forage include legumes such as alfalfa, as well as forage grasses such as bromegrass, and bluestem. In some cases, the plant is a Brassicaceae or other Solanaceae species. In some embodiments, the plant is not a species of Arabidopsis, for example, in some embodiments, the plant is not Arabidopsis thaliana.

Additional examples of hosts cells and host organisms include, without limitation, tobacco cells such as Nicotiana benthamiana, Nicotiana tabacum, Nicotiana rustica, Nicotiana excelsior, and Nicotiana excelsiana cells; cells of the genus Escherichia such as the species Escherichia coli; cells of the genus Clostridium such as the species Clostridium ljungdahlii, Clostridium autoethanogenum or Clostridium kluyveri; cells of the genus Corynebacterium such as the species Corynebacterium glutamicum; cells of the genus Cupriavidus such as the species Cupriavidus necator or Cupriavidus metallidurans; cells of the genus Pseudomonas such as the species Pseudomonas fluorescens, Pseudomonas putida or Pseudomonas oleavorans; cells of the genus Delftia such as the species Delftia acidovorans; cells of the genus Bacillus such as the species Bacillus subtilis; cells of the genus Lactobacillus such as the species Lactobacillus delbrueckii; or cells of the genus Lactococcus such as the species Lactococcus lactis.

“Host cells” can further include, without limitation, those from yeast and other fungi, as well as, for example, insect cells. Examples of suitable eukaryotic host cells include yeasts and fungi from the genus Aspergillus such as Aspergillus niger; from the genus Saccharomyces such as Saccharomyces cerevisiae; from the genus Candida such as C. tropicalis, C. albicans, C. cloacae, C. guillermondii, C. intermedia, C. maltosa, C. parapsilosis, and C. zeylenoides; from the genus Pichia (or Komagataella) such as Pichia pastoris; from the genus Yarrowia such as Yarrowia lipolytica; from the genus Issatchenkia such as Issathenkia orientalis; from the genus Debaryomyces such as Debaryomyces hansenii; from the genus Arxula such as Arxula adenoinivorans; or from the genus Kluyveromyces such as Kluyveromyces lactis or from the genera Exophiala, Mucor, Trichoderma, Cladosporium, Phanerochaete, Cladophialophora, Paecilomyces, Scedosporium, and Ophiostoma.

In some cases, the host cells can have organelles that facilitate manufacture or storage of the terpenes, diterpenes, diterpenoid alkaloids, and terpenoids. Such organelles can include lipid droplets, smooth endoplasmic reticulum, plastids, trichomes, vacuoles, vesicles, plastids, and cellular membranes. During and after production of the terpenes, diterpenes, diterpenoid alkaloids, and terpenoids these organelles can be isolated as a semi-pure source of the of the terpenes, diterpenes, diterpenoid alkaloids, and terpenoids.

### Definitions

As used herein, the singular forms “a,” “an,” and “the” are intended to include the plural forms as well, unless the context clearly indicates otherwise. Also, as used herein, “and/or” refers to, and encompasses, any and all possible combinations of one or more of the associated listed items. Unless otherwise defined, all terms, including technical and scientific terms used in the description, have the same meaning as commonly understood by one of ordinary skill in the art to which this invention pertains.

The term “about”, as used herein, can allow for a degree of variability in a value or range, for example, within 10%, within 5%, or within 1% of a stated value or of a stated limit of a range.

The term “enzyme” or “enzymes”, as used herein, refers to a protein catalyst capable of catalyzing a reaction. Herein, the term does not mean only an isolated enzyme, but also includes a host cell expressing that enzyme. Accordingly, the conversion of A to B by enzyme C should also be construed to encompass the conversion of A to B by a host cell expressing enzyme C.

The term “heterologous” when used in reference to a nucleic acid refers to a nucleic acid that has been manipulated in some way. For example, a heterologous nucleic acid includes a nucleic acid from one species introduced into another species. A heterologous nucleic acid also includes a nucleic acid native to an organism that has been altered in some way (e.g., mutated, added in multiple copies, linked to a non-native promoter or enhancer sequence, etc.). Heterologous nucleic acids can include cDNA forms of a nucleic acid; the cDNA may be expressed in either a sense (to produce mRNA) or anti-sense orientation (to produce an anti-sense RNA transcript that is complementary to the mRNA transcript). For example, heterologous nucleic acids can be distinguished from endogenous plant nucleic acids in that the heterologous nucleic acids are typically joined to nucleic acids comprising regulatory elements such as promoters that are not found naturally associated with the natural gene for the protein encoded by the heterologous gene. Heterologous nucleic acids can also be distinguished from endogenous plant nucleic acids in that the heterologous nucleic acids are in an unnatural chromosomal location or are associated with portions of the chromosome not found in nature (e.g., the heterologous nucleic acids are expressed in tissues where the gene is not normally expressed).

The terms “identical” or percent “identity”, as used herein, in the context of two or more nucleic acids or polypeptide sequences, refer to two or more sequences or subsequences that are the same or have a specified percentage of amino acid residues or nucleotides that are the same (e.g., at least 75% identity, 80% identity, 85% identity, 90% identity, 95% identity, 96% identity, 97% identity, 98% identity, 99% identity, or 100% identity in pairwise comparison). Sequence identity can be determined by comparison and/or alignment of sequences for maximum correspondence over a comparison window, or over a designated region as measured using a sequence comparison algorithm, or by manual alignment and visual inspection. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. A “reference sequence” is a defined sequence used as a basis for a sequence comparison; a reference sequence may be a subset of a larger sequence.

As used herein, a “native” nucleic acid or polypeptide means a DNA, RNA or amino acid sequence or segment that has not been manipulated in vitro, i.e., has not been isolated, purified, amplified and/or modified.

As used herein, the term “plant” is used in its broadest sense. It includes, but is not limited to, any species of grass (fodder, ornamental or decorative), crop or cereal, fodder or forage, fruit or vegetable, fruit plant or vegetable plant, herb plant, woody plant, flower plant or tree. It is not meant to limit a plant to any particular structure. It also refers to a unicellular plant (e.g. microalga) and a plurality of plant cells that are largely differentiated into a colony (e.g. volvox) or a structure that is present at any stage of a plant's development. Such structures include, but are not limited to, a seed, a tiller, a sprig, a stolen, a plug, a rhizome, a shoot, a stem, a leaf, a flower petal, a fruit, et cetera.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture.

As used herein, the term “plant part” as used herein refers to a plant structure or a plant tissue, for example, pollen, an ovule, a tissue, a pod, a seed, a leaf and a cell. Plant parts may comprise one or more of a tiller, plug, rhizome, sprig, stolen, meristem, crown, and the like. In some instances, the plant part can include vegetative tissues of the plant.

The terms “in operable combination,” “in operable order,” and “operably linked” refer to the linkage of nucleic acid sequences in such a manner that a nucleic acid molecule capable of directing the transcription of a coding region (e.g., gene) and/or the synthesis of a desired protein molecule is produced. The term also refers to the linkage of amino acid sequences in such a manner so that a functional protein is produced.

As used herein the term “terpene” includes any type of terpene or terpenoid, including for example any monoterpene, diterpene, sesquiterpene, sesterterpene, triterpene, tetraterpene, polyterpene, diterpenoid alkaloid, and any mixture thereof. In some cases the terpene is a diterpenoid alkaloid.

The term “transgenic” when used in reference to a plant or leaf or vegetative tissue or seed for example a “transgenic plant,” transgenic leaf,” “transgenic vegetative tissue,” “transgenic seed,” or a “transgenic host cell” refers to a plant or leaf or tissue or seed that contains at least one heterologous or foreign gene in one or more of its cells. The term “transgenic plant material” refers broadly to a plant, a plant structure, a plant tissue, a plant seed or a plant cell that contains at least one heterologous gene in one or more of its cells.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene.

The following non-limiting Examples describe some procedures that can be performed to facilitate making and using the invention.

### Example: Delphinium grandiflorum Enzymes for Diterpenoid Alkaloid Synthesis

Transcriptome sequencing was carried out on Delphinium grandiflorum, a plant from a neighboring genus to Aconitum. Transcriptome assembly both for D. grandiflorum and for three other Aconitum species (A. carmichaelii, A. japonicum, and A. vilmorinianum) allowed for comparative transcriptomics across tissue types and genera, leading to the identification of six enzymes active in this pathway. Furthermore, the public data for A. vilmorinianum—a root tissue time course study²²—allowed for coexpression analysis, where top hits were simply searched back against our own D. grandiflorum transcriptome for cloning and characterization. This resulted in the identification of a seventh enzyme active in the pathway which has little homology to previously characterized enzymes.

This work demonstrates the utility of analyzing public data to augment the analysis of a single transcriptome, as the availability of these data were involved in the identification of five out of the seven enzymes discovered.

### A. Materials and Methods

1. Plant Material, RNA Isolation, and cDNA Synthesis

D. grandiflorum plants were grown in a greenhouse under ambient photoperiod and 24° C. day/17° C. night temperatures. RNA isolation from flowers, leaves, and roots, quality assessment, RNA sequencing, and cDNA synthesis was carried out as described in Miller et al. 2020²⁸ (in parallel with samples prepped for L. frutescens; see Miller et al. Chapter 2).

2. D. Grandiflorum and Aconitum Genera De Novo Transcriptome Assembly and Analysis

RNA-seq data were obtained through RNA sequencing on an Illumina HiSeq 4000 for D. grandiflorum and the NCBI Sequence Read Archive (see website ncbi.nlm.nih.gov/sra) for A. carmichaelii (PRJNA415989)²⁴, A. japonicum (PRJDB4889), and A. vilmorinianum (PRJNA667080)²². Transcriptome assembly and analysis was carried out exactly as described in Miller et al. 2020²⁸ (see Chapter 2), with the exception of adaptor trimming, which was done with TrimGalore (v0.6.5; see webpage: github.com/FelixKrueger/TrimGalore). CD-HIT (v4.8.1)^{50,51} was used for clustering of D. grandiflorum P450 sequences. Sequence similarity networks were made with BLAST (v2.7.1+) and visualized with Cytoscape 52.

Initial assembly of the D. grandiflorum transcriptome resulted in incomplete transcripts for DgrTPS1 and DgrTPS7 (only ˜75% coverage of reference sequences), and although this was prior to our characterization of these enzymes, we noted that these transcripts were most likely misassembled given their high expression and likelihood of being involved in the pathway. Reassembly of the D. grandiflorum transcriptome was therefore done with only data acquired from root tissue, with reads from each tissue type mapped to this assembly. Transcripts for both of these genes in the new assembly aligned to the entire length of reference sequences, and so this assembly was used for further analysis.

3. Coexpression Analysis

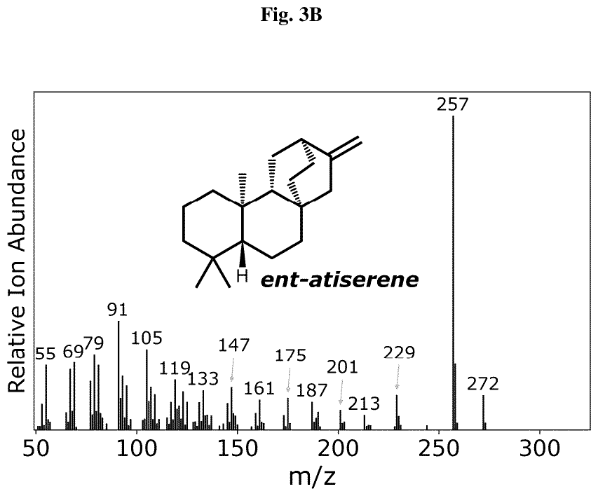

Our assembly for A. vilmorinianum was used for coexpression analysis. To minimize the computational burden, we reduced the analysis through clustering by 99% identity with CD-HIT (v4.8.1)^{50,51}, calculated expression levels through mapping reads to this clustered transcriptome, and eliminated any transcript with no samples that had at least 20% the expression level (in TPM) as any sample for either TPS. Coexpression analysis was carried out as described by Wisecaver et al. 2017⁴³ (pipeline at: see website github.itap.purdue.edu/jwisecav/mr2mods). The resulting coexpression network shown in FIG. 3.10 shows only genes with one or two degrees of separation from any of the first four genes in the pathway (DgrTPS1, DgrTPS7, CYP701A127, and CYP71FH1) based on a mutual rank (MR) cutoff of e\hat{ }(−(MR-1)/5)>0.01. Orthologs from each transcriptome were found with BLAST (v2.7.1+) and visualized with Cytoscape 52.

4. Cloning

PCR amplification from cDNA, cloning, and constructs used for transient expression in N. benthamiana were carried out as described in Miller et al. 2020²⁸ for plastidial tests with GGPP (see Chapter 2). Constructs for ZmAN2, NmTPS1, and NmTPS2 in pEAQ (used as positive controls for ent-CPP, (+)-CPP, and ent-kaurene biosynthesis, respectively) were made by Johnson et al. 2019⁵³.

5. Transient Expression in N. benthamiana, Product Scale-Up, and NMR Analysis

Transient expression in N. benthamiana for screening assays was carried out exactly as described in Miller et al. 2020²⁸ (see Chapter 2), with the exception of solvents used to extract each set of assays as described in the main text. For ent-atiserene and ent-atiserene-20-al scaleup, three whole plants were infiltrated with a syringe, and approximately 15/30 g of fresh weight were extracted with hexane/ethyl acetate (respectively). Products were purified through silica chromatography with 10% ethyl acetate: 90% hexane as the mobile phase. NMR analysis was carried out on a Bruker 800 MHz spectrometer equipped with a TCl cryoprobe using CDCl₃ as the solvent. CDCl₃ peaks were referenced to 7.26 and 77.00 ppm for ¹H and ¹³C spectra, respectively.

6. GC-MS Analysis

All GC-MS analyses were performed on hexane or ethyl acetate extracts (described for each case in the text) with an Agilent 7890A GC with an Agilent VF-5 ms column (30 m×250 μm×0.25 μm, with 10 m EZ-Guard) and an Agilent 5975C detector. The inlet was set to 250° C. splitless injection of 1 μL, He carrier gas (1 ml/min), and the detector was activated following a 3 min solvent delay. The following method was used for analysis of each sample presented in the text: temperature ramp start 40° C., hold 1 min, 40° C./min to 200° C., hold 2 min, 20° C./min to 280° C., 40° C./min to 320° C.; hold 5 min. Figures for chromatograms and mass spectra were generated with Pyplot.

7. LC-MS Analysis

All LC-MS analyses were performed on 80% methanol: 20% H₂O N. benthamiana extracts with a Waters Xevo G2-XS quadrupole ToF UPLC with a Waters ACQUITY C18 (2.1×100 mm) column and an injection of 10 μL. The following method was used for analysis of each sample presented in the text: Initial 99% Solvent A (10 mM ammonium formate [pH2.8]): 1% Solvent B (acetonitrile), continuous gradient to 2% A: 98% B over 12 min, hold for 1.5 min, continuous gradient to 99% A: 1% B over 0.1 min, hold 1.5 min. Figures for chromatograms and mass spectra were generated with Pyplot.

TABLE 2
¹H and ¹³C chemical shifts for ent-atiserene. CDCl₃ peaks were
referenced to 7.26 and 77.00 ppm for ¹H and ¹³C spectra,
respectively.

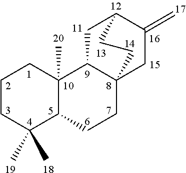

| ¹³C NMR | δ | ¹H NMR | δ |
|---|---|---|---|
| C1 | 39.4 | 2H | 0.79; 1.53 |
| C2 | 18.2 | 2H | 1.38; 1.59 |
| C3 | 42.2 | 2H | 1.15; 1.38 |
| C4 | 33.1 | — | — |
| C5 | 56.3 | 1H | 0.82 |
| C6 | 18.8 | 2H | 1.34; 1.48 |
| C7 | 39.5 | 2H | 1.14; 1.18 |
| C8 | 33.5 | — | — |
| C9 | 52.8 | 1H | 1.16 |
| C10 | 37.7 | — | — |
| C11 | 28.6 | 2H | 1.42; 1.59 |
| C12 | 36.6 | 1H | 2.24 |
| C13 | 28.7 | 2H | 0.99; 1.94 |
| C14 | 27.4 | 2H | 1.59; 1.62 |
| C15 | 48.3 | 2H | 1.91; 2.05 |
| C16 | 153.2 | — | — |
| C17 | 104.4 | 2H | 4.58; 4.74 |
| C18 | 33.5 | 3H | 0.87 |
| C19 | 21.7 | 3H | 0.84 |
| C20 | 13.9 | 3H | 0.98 |

### Results

1. Initial Biosynthetic Pathway

The majority of diterpenoid alkaloids in the Ranunculaceae family can be divided into two major groups based on the number of carbons in their backbone structure (20 or 19) and ring structure (6/6/6/6 or 6/7/5/6, respectively) 13,14. Despite these differences, the inventors proposed that both major groups are derived from the same diterpene starting scaffold. Two examples—the complex structure aconitine and a simple C20 hetidine-type diterpenoid alkaloid—are shown in Scheme 1 described above (reproduced below), and three structural features of these metabolites suggest a common origin. First, the cyclization pattern matches that of a class II TPS mechanism, with identical stereochemistry at three chiral centers indicated in shaded circles in Scheme 1, suggesting the involvement of an ent-copalyl diphosphate (ent-CPP) synthase. Second, tracing from the same carbon in both examples shows two three-carbon bridges making up two sides of a six-membered ring, similar to the structure of ent-atiserene²⁹. Third, the nitrogen is covalently bonded to the same methyl groups of the ent-atiserene backbone, indicating oxidative functionalization of the same two methyl groups—likely carried out by a pair of cytochrome P450s.

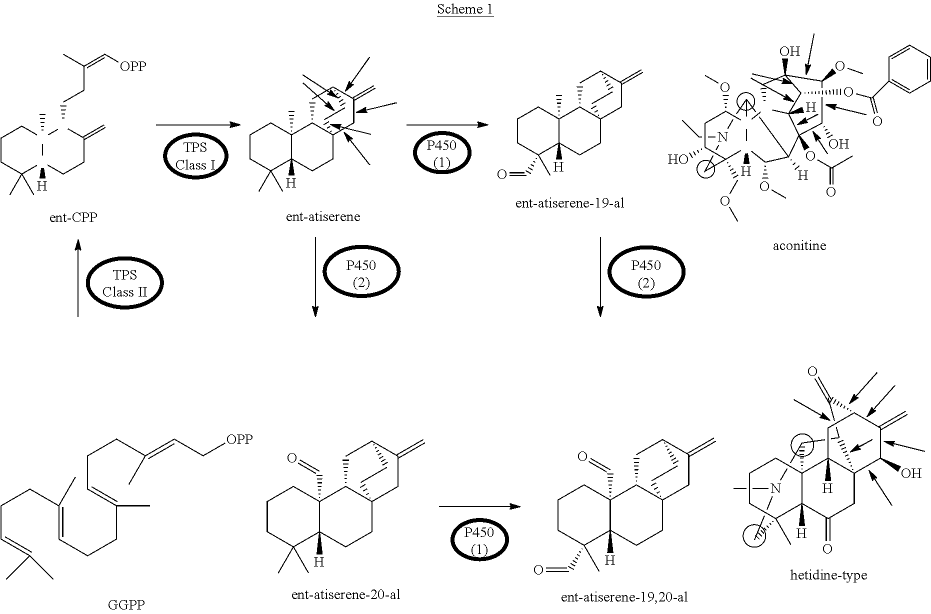

In Scheme 1, common structural features of diterpenoid alkaloids and proposed biosynthetic pathway are shown. Bonds shaded in gray have a common labdane structure likely derived from activity of a class II TPS (shown as a dotted line in aconitine due to a ring expansion proposed to happen further in the pathway). Carbons highlighted in shaded circles have common stereochemistry. Bonds with arrows show the same three-carbon bridges that make up either side of a six-membered ring. Carbons in open circles represent methyl groups on ent-atiserene which are likely converted to aldehydes to allow for nitrogen incorporation.

The proposed intermediate ent-atiserene-19-al closely resembles the central metabolite ent-kaurenoic acid—a key intermediate in the central metabolic pathway towards gibberellins³⁰—which is synthesized from GGPP through the activity of a class II/class I TPS pair and a cytochrome P450³⁰. Given these similarities, it is plausible that the genes responsible for making ent-atiserene-19-al are recent duplicates of these central metabolism enzymes, especially given the occurrence of polyploidization within the Delphinieae tribe (containing Aconitum and Delphinium) of the Ranunculaceae family³¹⁻³³.

2. RNA Sequencing and Transcriptome Assembly

Diterpenoid alkaloids primarily accumulate in root tissue throughout species in Aconitum and Delphinium³⁴⁻³⁷. RNA from D. grandiflorum was isolated and sequenced from the roots, leaves, and flowers to allow for comparative transcriptomics across tissue types. Furthermore, a wealth of public RNA sequencing data has been submitted to the NCBI Sequence Read Archive (SRA) for the Aconitum genus, and three datasets from A. carmichaelii (root, leaf, flower, bud; PRJNA415989)²⁴, A. japonicum (root, root tuber, leaf, flower, stem; PRJDB4889), and A. vilmorinianum (root timecourse; PRJNA667080) 22 were included as well. Transcriptomes for each species were assembled, allowing for multiple cross-tissue and cross-species comparisons to search for genes involved in diterpenoid alkaloid metabolism.

3. A Pair of TPSs Cyclizes GGPP to Ent-Atiserene

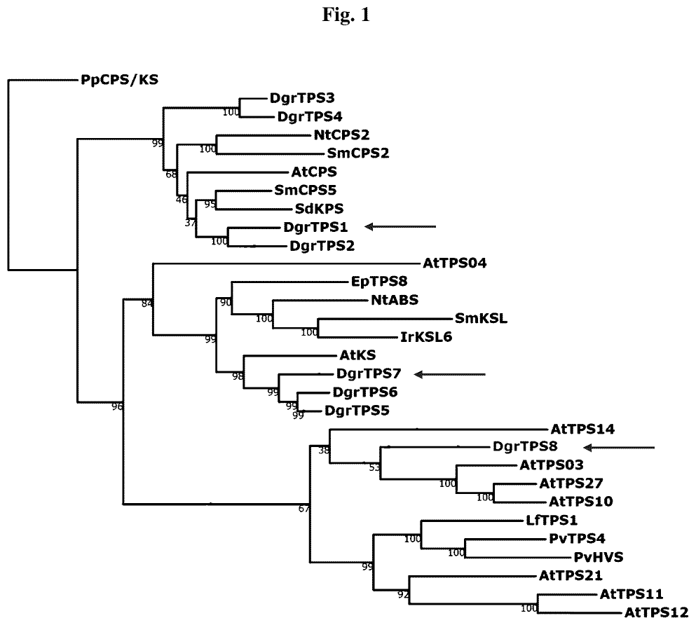

The first two steps in this pathway were proposed to be a pair of TPSs; first a class II TPS that converts GGPP to ent-CPP, and second a class I TPS which converts ent-CPP to ent-atiserene. At this stage, only the D. grandiflorum transcriptome had been assembled, and following analysis of this transcriptome, candidates were characterized without the need for data from the three other Aconitum species. A BLAST search of the D. grandiflorum transcriptome against a reference set of plant TPSs revealed fifteen putative TPS genes. Only three of these were exclusively expressed in root tissue, matching the tissue-specific accumulation of diterpenoid alkaloids. Phylogenetic analysis revealed that these belonged to the TPS-c, TPS-e, and TPS-b subfamilies (FIG. 1). DgrTPS1 (TPS-c) and DgrTPS7 (TPS-e) appeared to be the most likely candidates, as they belong to the pair of subfamilies typically implicated in labdane-related diterpene biosynthesis. Furthermore, their closest paralogs (DgrTPS2 and DgrTPS5/6, respectively) have low expression across all three tissues, as would be expected for the pair of TPSs involved in central metabolism for gibberellin biosynthesis.

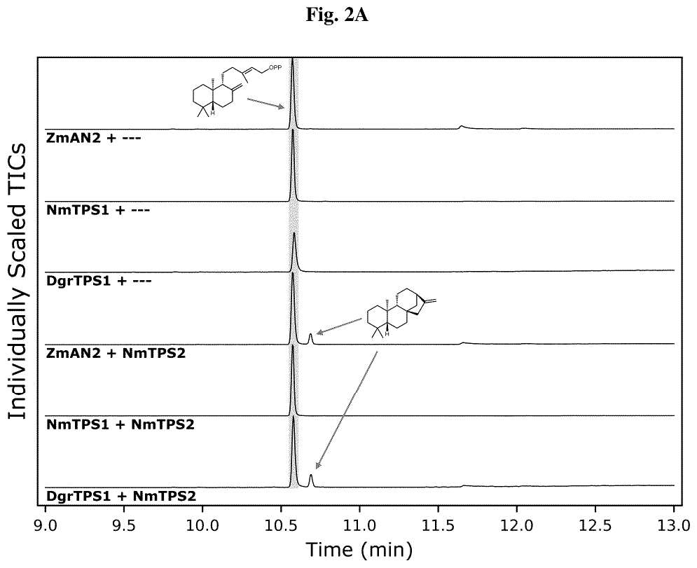

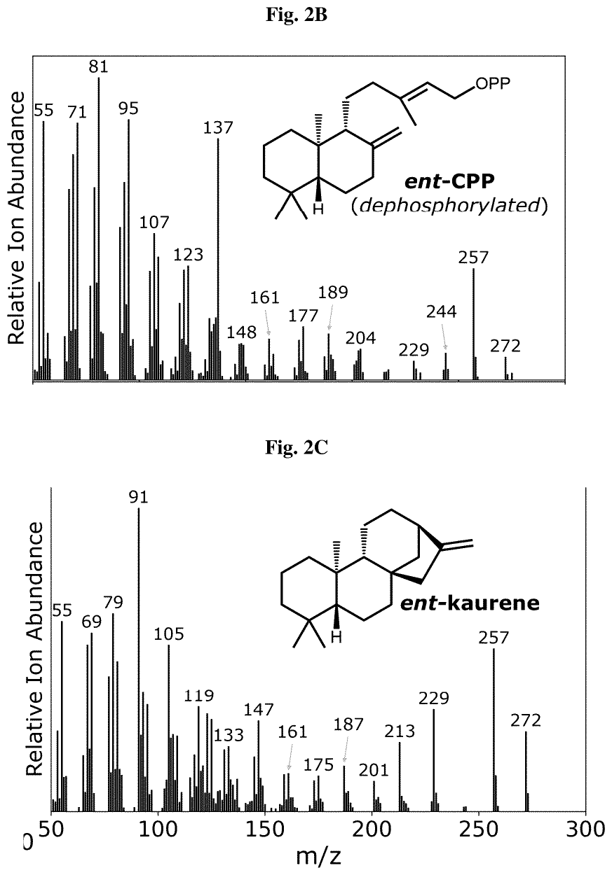

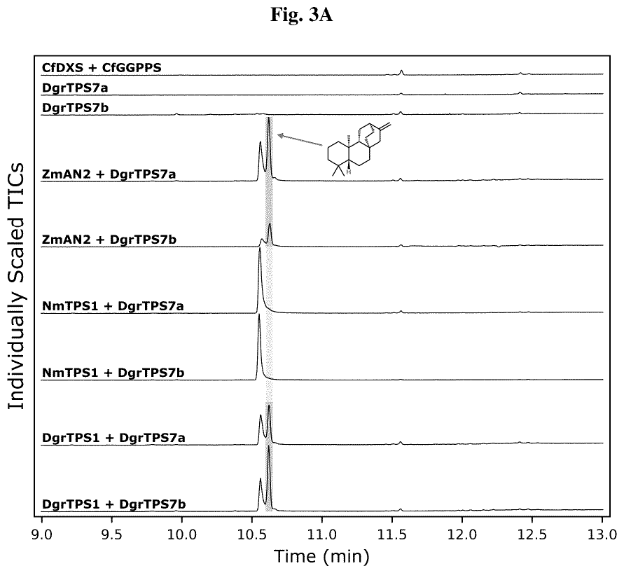

Full-length genes for DgrTPS1 and DgrTPS7 were cloned from D. grandiflorum root cDNA into pEAQ for transient expression in N. benthamiana. Two isoforms of DgrTPS7, not distinct in our transcriptome assembly, were cloned from cDNA, and both were tested (named DgrTPS7a/7b). All screening through transient expression in N. benthamiana throughout this chapter included coexpression with CfDXS and CfGGPPS (to increase precursor supply of GGPP 38). The CfDXS is a Plectranthus barbatus 1-deoxy-D-xylulose 5-phosphate synthase (genbank accession: KP889115) and the CfGGPPS is a geranylgeranyl diphosphate synthase (genbank accession: KP889114). GC-MS analysis on hexane extracts revealed that of DgrTPS1 acts as a copalyl diphosphate (CPP) synthase, the absolute stereochemistry of which was established as ent-CPP through coexpression with an enantioselective ent-kaurene synthase (NmTPS2) (FIGS. 2A-2C).

Following this result, DgrTPS7a/7b was tested and showed conversion of ent-CPP to a new product with a fragmentation pattern matching that of ent-atiserene 29 for both isoforms (FIGS. 3A-3B). To confirm the identity of this new product as ent-atiserene, transient expression in N. benthamiana was scaled up with DgrTPS1 and DgrTPS7a, and the product was purified through silica chromatography and confirmed through NMR (See Table 2 below). Since both isoforms of DgrTPS7 were shown to have the same function, DgrTPS7a was used for further testing and is simply referred to as DgrTPS7 throughout the remainder of this chapter.

4. Two Pairs of Cytochrome P450s with Overlapping Functions Oxidize Ent-Atiserene

Following the confirmation that a pair of terpene synthases make ent-atiserene, we continued with our proposed biosynthetic pathway to search for cytochrome P450s which can carry out sequential oxidations of methyl groups 19 and 20 to aldehydes. In contrast to the TPS family, the identification of P450s presents a challenge due to the number of genes that may be present in any given plant³⁹. In our transcriptome assemblies for D. grandiflorum and the three Aconitum species, a BLAST search against a reference set of P450 sequences yielded 2,061 predicted P450 transcripts. For D. grandiflorum alone, there were 297 after clustering shorter transcripts with greater than 95% sequence identity.

To narrow this down to a manageable number to test, a similar strategy to our previous work in identifying the P450 involved in the leubethanol pathway (Chapter 2) 28 was used by taking advantage of the assumed conservation of this pathway between neighboring genera and tissue-specific accumulation of metabolites. The total transcripts from each assembly were first assigned to individual clans based on homology to the closest reference sequence, and individual phylogenies were made for distinct clans. The transcripts were filtered to include only those in D. grandiflorum with high root expression and with a root-expressed ortholog in each Aconitum assembly. This narrowed down a list of 297 possible P450s to just 7 to test.

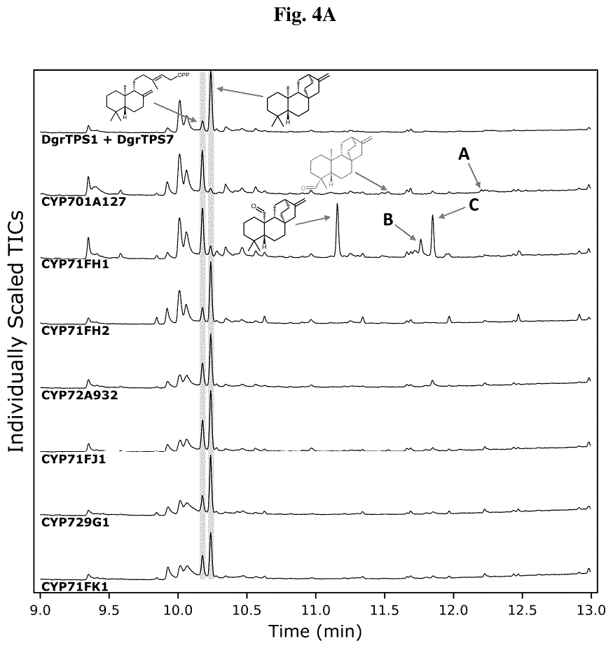

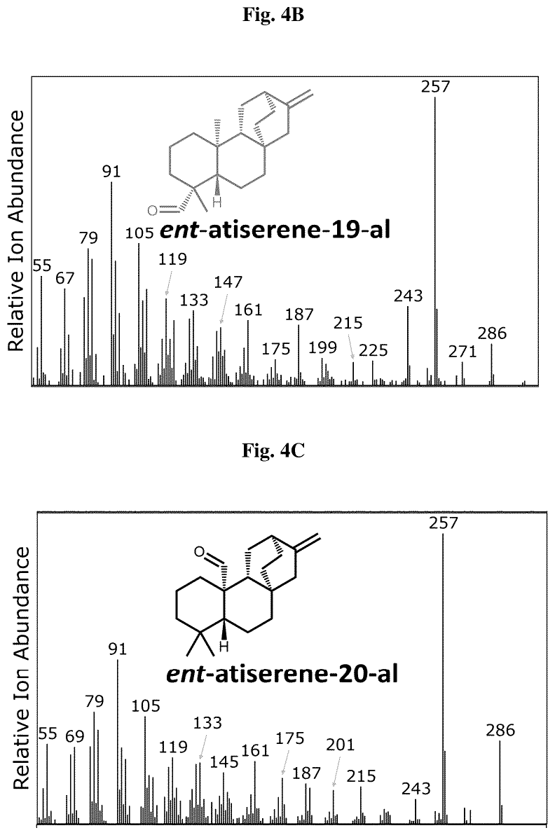

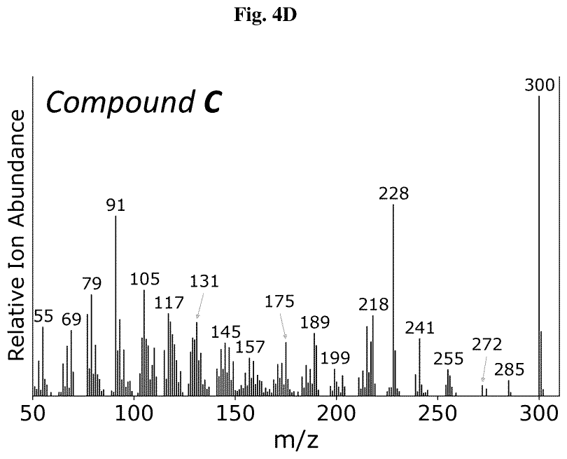

These seven P450s were cloned from D. grandiflorum root cDNA and tested through transient expression in N. benthamiana. Each candidate was coexpressed with DgrTPS1 and DgrTPS7, and products were analyzed via GC-MS following ethyl acetate extraction. CYP701A127 and CYP71FH1 both showed activity in oxidizing the ent-atiserene backbone (FIGS. 4A-4D). Coexpression with either of these P450s showed a depletion in ent-atiserene and the production of respective metabolites with an m/z value of 286 and retention of 257 m/z as the highest abundance fragment ion (FIGS. 4A-4D), consistent with sequential oxidations of a methyl group to an aldehyde. Both enzymes also made a product with an m/z value of 302 (compounds A and B; FIG. 4A), consistent with either a third oxidation of this carbon to an acid or addition of another hydroxyl group elsewhere. CYP71FH1 also produces a major product with an m/z value of 300 (compound C; FIG. 4D), which would suggest a net addition of two oxygen atoms and four oxidations from ent-atiserene.

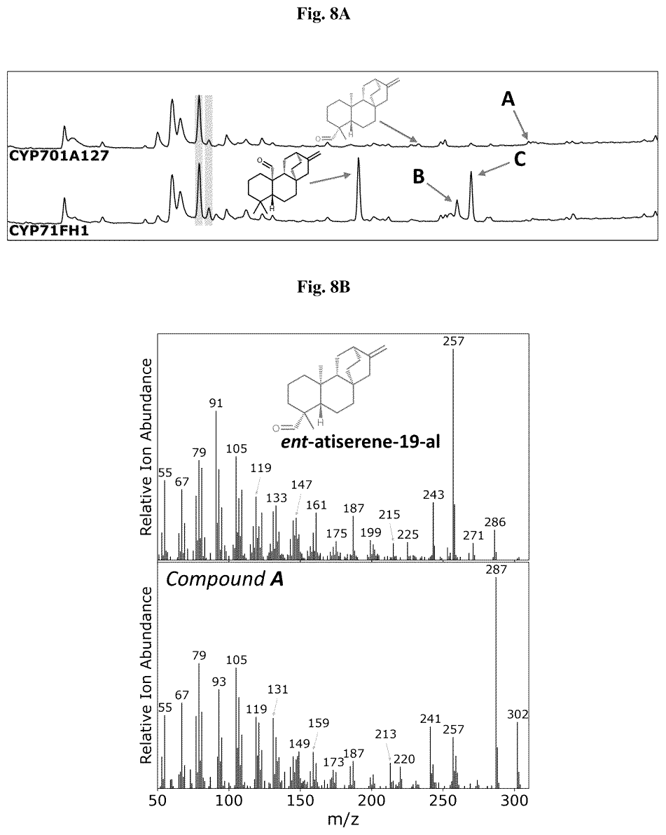

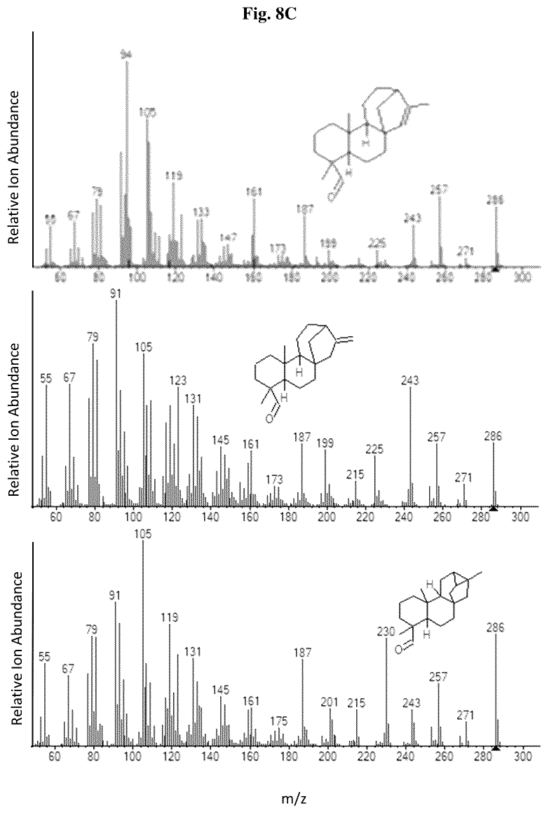

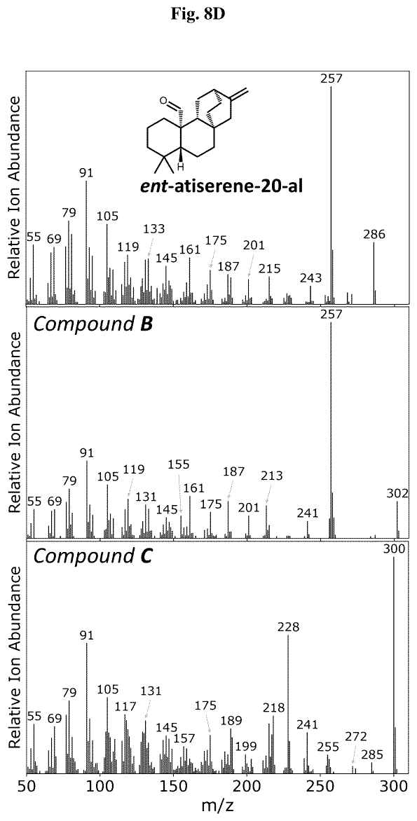

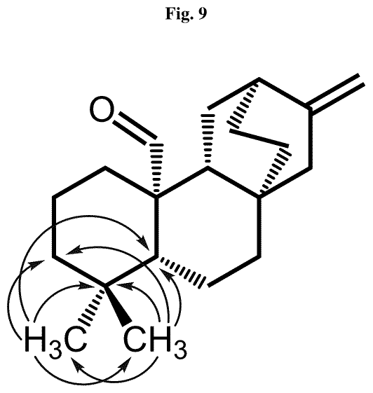

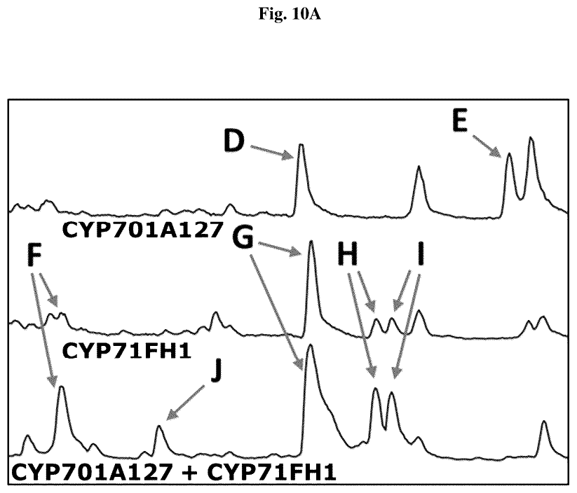

For the products of CYP71FH1, production was scaled up in N. benthamiana to purify compounds and attempt to solve structures by NMR. While sufficient quantities were simple to produce through expression and extraction from approximately 30 g of fresh weight, purification of the two major products from each other proved challenging. One fraction purified through a silica column was sufficiently enriched for the 286 m/z product that its identity was confirmed as ent-atiserene-20-al through NMR. For the products of CYP701A127, they may have been poorly detectable by GC or shuttled away to other products through conversion by endogenous N. benthamiana enzymes. CYP701A127's product was tentatively assigned as ent-atiserene-19-al based on the mass spectrum both in terms of its own fragmentation pattern and in comparison to similar structures in the NIST database (FIGS. 8A-8B), close retention time to ent-atiserene-20-al, and phylogenetic evidence that CYP701A127 is a recent duplication of its putative central metabolism paralog (likely an ent-kaurene oxidase that oxidizes this same carbon).

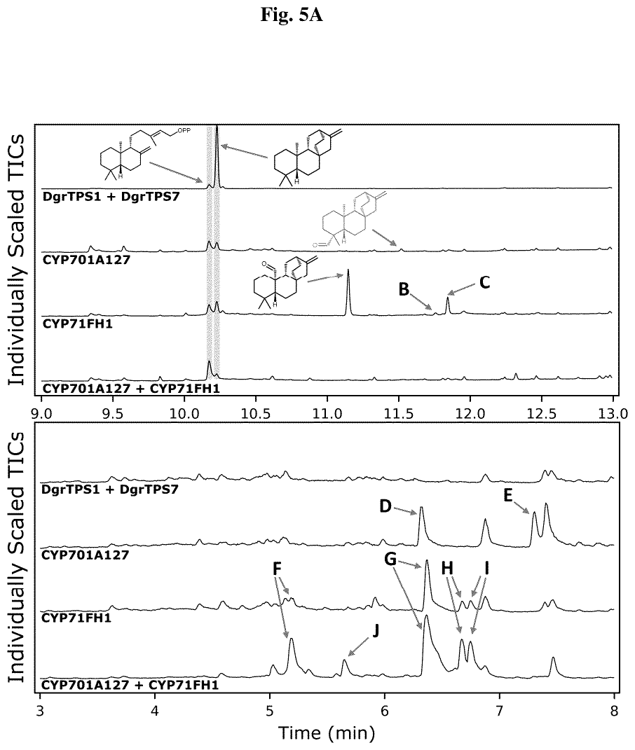

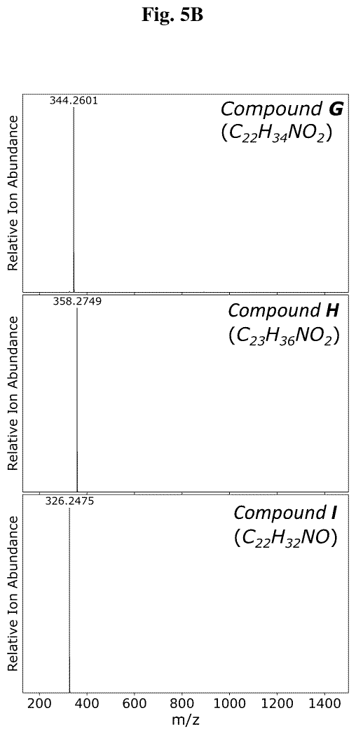

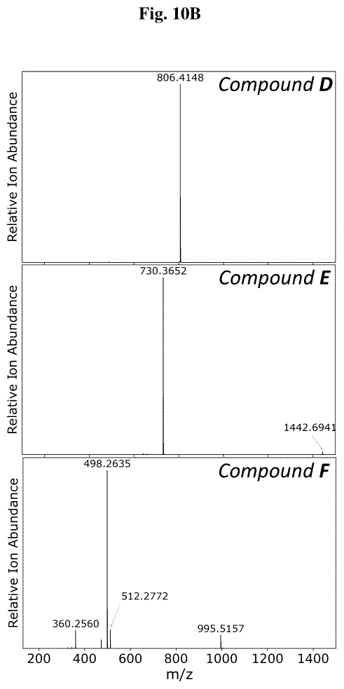

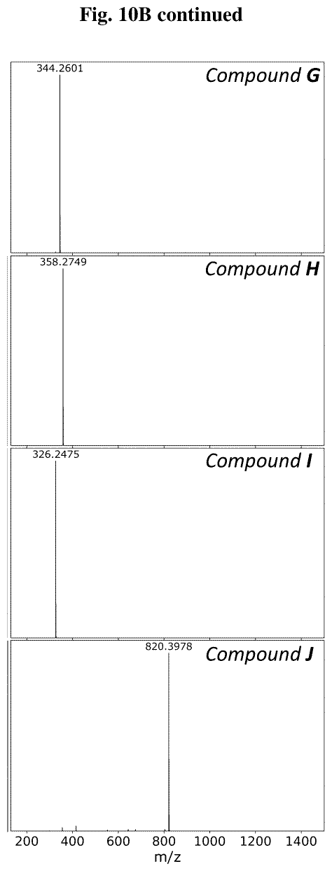

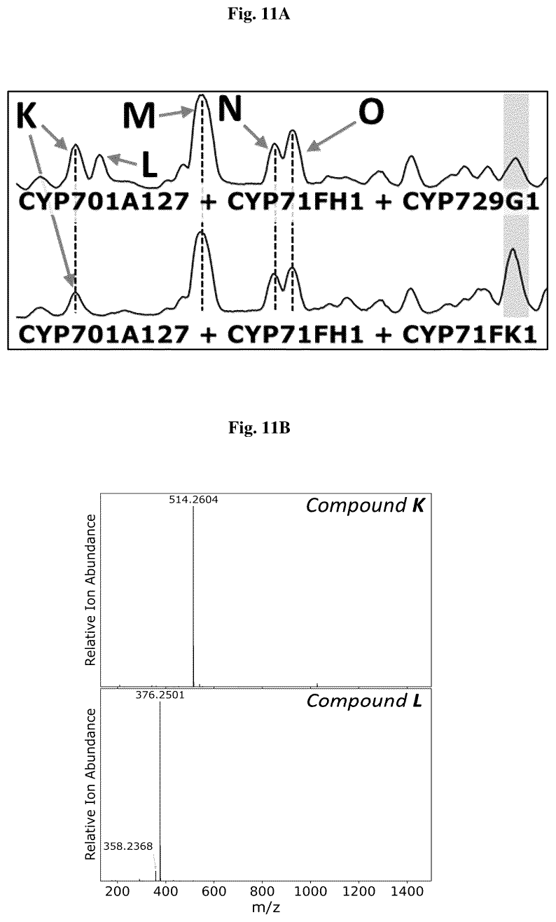

In our proposed biosynthetic pathway, a pair of P450s could work together to oxidize both methyl groups at carbons 19 and 20 to aldehydes, and so whether coexpression of both of these enzymes would further the pathway was tested. Ethyl acetate extraction and GC-MS analysis on both TPSs and P450s coexpressed revealed a depletion of both ent-atiserene and of both P450's respective products (FIGS. 5A-5B). These assays were also analyzed by LC-MS on 80% methanol extracts, which revealed two products from CYP701A127 (compounds D and E), four from CYP71FH1 (compounds F-I), and a total of five products with coexpression of both enzymes (FIGS. 5A-5B and FIGS. 10A-10B). Four of the products present with both P450s coexpressed are an accumulation of CYP71FH1's products (compounds F-I, including its major product G), suggesting that these are products different than those detected by GC-MS for CYP71FH1 alone, and that CYP701A127 may share a partial functional redundancy with CYP71FH1. One additional minor product is present (compound J) when both are coexpressed.

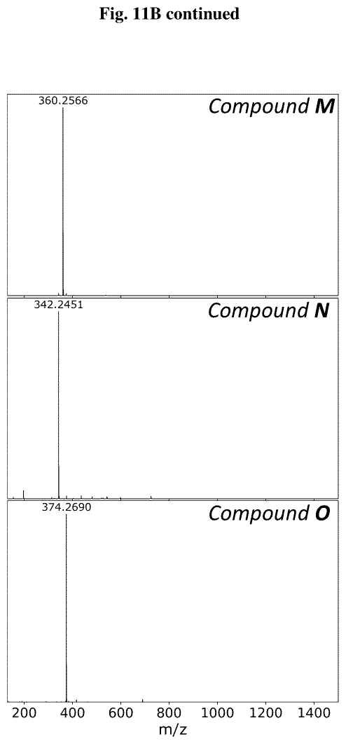

This pair of P450s was further characterized against the remaining five candidates. Coexpression of both TPSs, both P450s, and each remaining P450 candidate revealed that both CYP729G1 and CYP71FK1 can act on these products (FIG. 6A and FIGS. 11A-11B). The molecular ions for each product suggest that they are each a single hydroxylation difference (additional ˜16 m/z) from major products for CYP701A127 and CYP71FH1 alone. Interestingly, despite these enzymes being evolutionarily distant (belonging to entirely different clans), both give the same product profile, with the exception of one additional product present with coexpression of CYP729G1 (compound L) which is not present with CYP71FK1.

5. Continuation of the Previously Proposed Biosynthetic Pathway

Rather than stop to identify every possible intermediate, we chose to continue with the pathway through screening additional candidates. Accumulation of intermediates and side products is likely to occur when pathways are incompletely reconstructed or artificially altered^{3,40}, and the abundance of products from these four P450s may be due to an accumulation of intermediates which would not occur with the coexpression of subsequent steps in the pathway.

Considering that CYP701A127 and CYP71FH1 carry out the oxidations proposed in the initial biosynthetic pathway required for nitrogen incorporation, as described herein, this incorporation likely follows these two steps. In many alkaloid biosynthetic pathways, the formation of an alkaloid scaffold involves the accumulation of both an amine and aldehyde precursor⁹. The nitrogen present in the majority of diterpenoid alkaloids in Aconitum and Delphinium may be derived from ethylamine due to the attached —CH₂CH₃ group (FIG. 3.9), while some metabolites presumably incorporate methylamine (—CH₃) or ethanolamine (—CH₂CH₂OH)^{13,14}—the origin of which could come from decarboxylation of alanine, glycine, or serine, respectively. Serine decarboxylases are present in central metabolism, and a duplication of this enzyme in Camellia sinensis has been shown to decarboxylate alanine into ethylamine (AlaDC) in theanine biosynthesis⁴¹. Additionally, Spirea japonica—an evolutionarily distinct plant which makes similar compounds—has been shown to produce isotopically labeled diterpenoid alkaloids through addition of labeled serine⁴².

The mechanism of nitrogen incorporation is also an important consideration, as the iminium cation formed through condensation of an amine and aldehyde is inherently unstable. Quenching of this cation through either a substitution or reduction⁹ can avoid spontaneous hydrolysis separating them back into their constituent parts, and in the case of diterpenoid alkaloids, it likely follows both mechanisms based on the number of bonds present on both oxidized methyl groups (Scheme 2 below). Carbon 20 almost always contains an extra carbon-carbon bond relative to ent-atiserene and the intermediate ent-atiserene-20-al, while carbon 19 does not, similar to both ent-atiserene and the intermediate ent-atiserene-19-al. This suggests that incorporation at carbon 19 requires a reductase, and at carbon 20 may involve a spontaneous intra-molecular condensation.

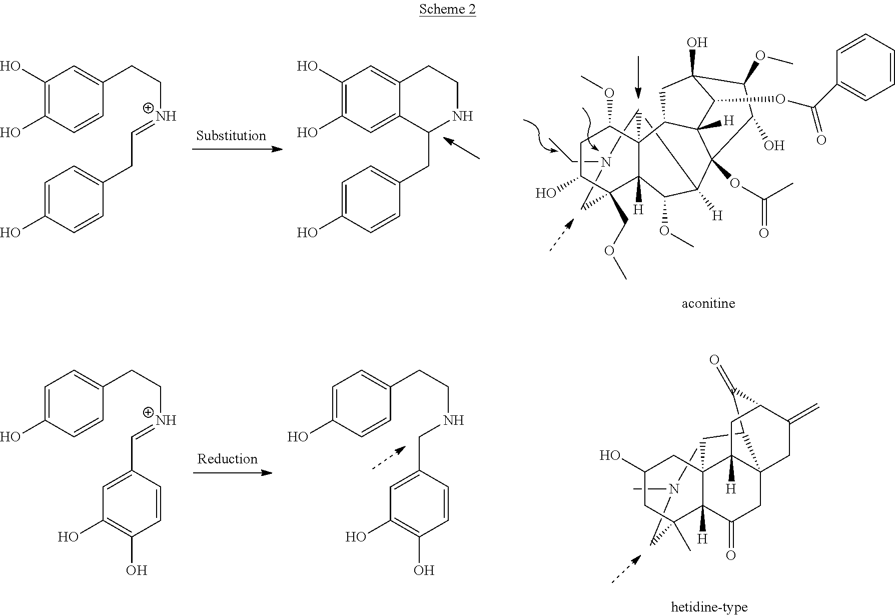 [SMILES: Oc1ccc(C/C=[NH+]/CCc2ccc(O)c(O)c2)cc1 ; Oc1ccc(CC2NCCc3cc(O)c(O)cc32)cc1 ; CCN1CC2[C@@H]3[C@H](OC)[C@@H]4[C@](COC)(C1)[C@H](O)C[C@H](OC)[C@]24[C@@H]1C[C@@]2(O)[C@H](OC(=O)c4ccccc4)[C@@H]1[C@]3(OC(C)=O)[C@@H](O)[C@@H]2OC ; Oc1ccc(CC/[NH+]=C/c2ccc(O)c(O)c2)cc1 ; Oc1ccc(CCNCc2ccc(O)c(O)c2)cc1 ; C=C1C[C@]23CC(=O)[C@@H]4[C@@]5(C)CC(O)C[C@]46C(C2C(=O)[C@H]1C[C@H]36)N(C)C5]

In Scheme 2 illustrated above, nitrogen incorporation into diterpenoid alkaloids likely involves iminium cation resolution through reduction and substitution. In the example on the left, highlighted by Lichman 2021⁹, showing how the iminium cation in norcoclaurine biosynthesis is resolved through substitution (top substitution reaction), while similar compounds from the Amaryllidaceae family involve a reduction (bottom reduction reaction). On the right, representative compounds from Delphinium and Aconitum with solid or dashed arrows pointing to carbons corresponding to the proposed reaction mechanism shown on examples on the left (substitution=solid arrow; reduction=dashed arrow). The two curved arrow point to the of aconitine proposed here to originate from ethylamine—present in the majority of diterpenoid alkaloids.

In contrast to the steps elucidated thus far, involving carbocation-mediated cyclizations (TPSs) and site-specific oxidations (P450s), the reaction of an amine and aldehyde to form an alkaloid scaffold could occur either spontaneously or through enzyme catalysis given the inherent reactivity between aldehydes and primary amines. The putative involvement of a reductase is also not straightforward in terms of how many different enzyme families this function could evolve from. To search for the next step(s), coexpression analysis was carried out to determine which genes were coexpressed with the first four enzymes already found in the pathway (DgrTPS1, DgrTPS7, CYP701A127, and CYP71FH1).

This analysis was carried out on public data. The data collected for A. vilmorinianum involved sequencing three replicates of root tissue at three different stages of development²², and so coexpression analysis was carried out on this dataset and BLAST searched the top hits back against our set of four transcriptomes. A coexpression network showing all A. vilmorinianum genes coexpressed with the respective orthologs of the first four steps characterized in the pathway were the anchor sequences. Nodes represented assembled transcripts and edges represent coexpression between genes determined by mutual rank (MR; cutoff: e\hat{ }(−(MR-1)/5)>0.01)43. Genes included in this network either meet this threshold with one of the anchor sequences or with another gene that does (i.e. two degrees of separation). Nodes further from the center represented genes that meet this coexpression threshold with a greater number of anchor sequences; nodes in the center do not meet the cutoff threshold directly with any anchor sequence. Four candidates were selected for characterization.

Three putative reductases were found which were highly coexpressed with the A. vilmorinianum orthologs of our four initial pathway genes, and one putative cupin (named here simply as VGCRed, OxoRed, SangRed, and Cupin, respectively).

6. Coexpression Analysis Reveals that a Predicted Reductase is Active in the Pathway

Each of these four genes were cloned from D. grandiflorum root cDNA and tested for activity through transient expression in N. benthamiana. The alanine decarboxylase (AlaDC) from C. sinensis 41 was also included to supply ethylamine to the pathway, both to see if new metabolites spontaneously form with our aldehyde intermediates and to ensure that our coexpression candidates, if required, have access to ethylamine. Testing of each candidate was carried out along with either the first four enzymes (DgrTPS1, DgrTPS7, CYP701A127, and CYP71FH1) or these four plus CYP729G1.

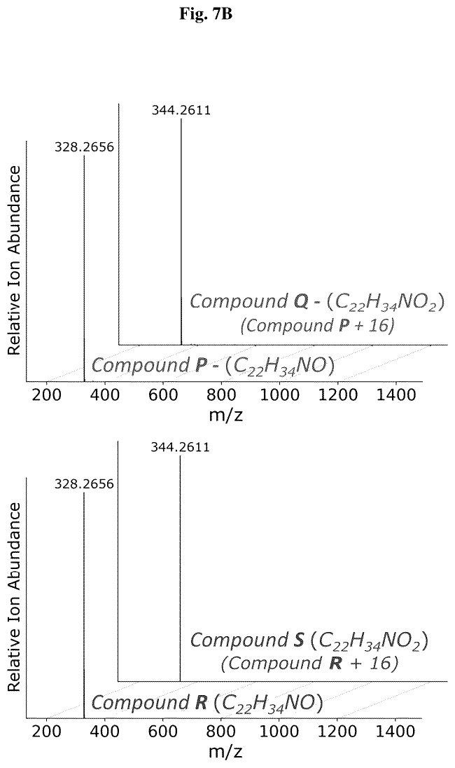

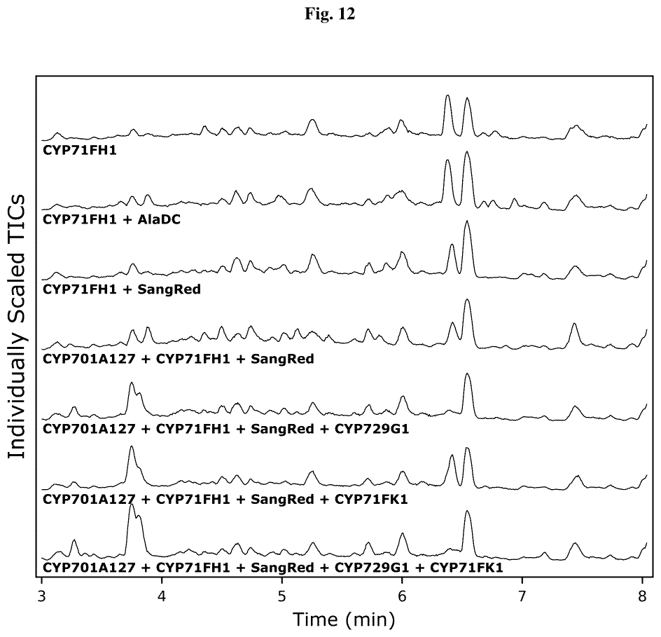

Two major results came from coexpression of these candidates with the first four enzymes (FIG. 7A-7B). First, coexpression of AlaDC resulted in a minor product with a proposed chemical formula of C₂₂H₃₃NO (exact mass 328.2647 in ESI+). Second, coexpression of SangRed led to nearly a complete depletion in precursors and the formation of a new peak with an exact mass identical to the minor product from AlaDC. Coexpression of SangRed along with the first four steps and CYP729G1 did not deplete all of CYP729G1's products. However, such coexpression did lead to the formation of a new peak with a proposed formula of C₂₂H₃₃NO₂ (exact mass 344.2611 in ESI+), suggesting that both of these enzymes compete for the products of the first four enzymes, while CYP729G1 can still hydroxylate the product of SangRed (or conversely that SangRed can convert the product of CYP729G1). Similar to the previous results with just the first four enzymes, coexpression with AlaDC led to the formation of a minor product with an identical exact mass (344.2611). Coexpression of both AlaDC and SangRed together along with the first four enzymes (or also CYP729G1) did not lead to an obvious increase in SangRed products, suggesting that ethylamine is not a substrate. Further testing revealed that SangRed produces its major product without the need for CYP701A127 and that CYP71FK1 retains its functional redundancy with CYP729G1, even in combination with SangRed (FIG. 12).

### C. Discussion

Through a combination of transcriptomics comparing tissue types and genera and coexpression analysis, seven enzymes active in the biosynthetic pathway towards diterpenoid alkaloids have been identified in the Ranunculaceae family. There are hundreds of diterpenoid alkaloids in this family, and the identification of these enzymes will serve as the basis for further pathway discovery towards specific metabolites. This work highlights the usefulness of utilizing public data as an orthogonal filter for selection of candidate enzymes beyond the analysis of a single species given the inherent complexity of these pathways.

One possible explanation for these assembly artifacts is that the genetics of members of the Delphinium and Aconitum genera are inherently complicated. Delphinium montanum, for example, is an autotetraploid with a predicted genome size of roughly 40 Gb³³ (2n=32⁴⁴). The four species studied here have a range of predicted ploidy levels (D. grandiflorum: 2n=16; A. carmichaelii: 2n=32/64— depending on cultivar; A. japonicum: 2n=32; A. vilmorinianum: 2n=16)⁴⁴, and it has been suggested that, at least in the Aconitum genus, there may have been multiple recent events of polyploidization and diploidization³². This fits with the model of our initial biosynthetic pathway—and the phylogenetic relationships of these genes—in which we predicted that the first three steps may be recent duplications of central metabolism enzymes given the similarity of these predicted intermediates to those in gibberellin biosynthesis³⁰. While we didn't characterize the putative central metabolism copies of these genes, Mao et al.²⁷ demonstrated a pair of recently-duplicated ent-CPP synthases and ent-kaurene/atiserene synthases in their analysis. CYP701A127, which we assigned as an ent-atiserene oxidase (making ent-atiserene-19-al) also belongs to the same family as CYP701A3, the ent-kaurene oxidase involved in central metabolism in Arabidopsis⁴⁵.

It should be noted that DgrTPS1—being an ent-CPP synthase—is technically not an enzyme which makes a specialized metabolite. Given its relative expression (˜75× higher in roots) over its putative central metabolism paralog (DgrTPS2), however, it is clearly dedicated to specialized metabolism. A similar phenomenon is seen in both Oryza sativa⁴⁶ and Zea mays⁴⁷, where two copies of an ent-CPP synthase are present; one which is involved in gibberellin biosynthesis and another which is inducible by pathogens for the production of defensive ent-CPP-derived specialized metabolites. Given the presence of duplicate ent-CPP synthases in each of these independent lineages of plants, there is likely a strong evolutionary pressure for the ability to tightly regulate these competing pathways.

Throughout the process, we varied the approach to identify each class of enzyme based on what information was necessary. For the terpene synthases, for example, few enough transcripts were present in our assembly that we relied solely on data from D. grandiflorum, as the choice of candidates to test was obvious given just this single dataset. For the P450s, the Aconitum datasets were essential given the presence of nearly 300 unique transcripts in our D. grandiflorum assembly. Had we not chosen to work with a neighboring genus, we may not have been able to filter candidates down to just seven that we tested, as the only orthologous genes present across each species in our analysis have persisted throughout roughly 27 million years since the speciation of the two genera⁴⁸. Notably, three of the P450s shown to be active are founding members of new subfamilies (denoted by the ending of “1”). Finally, even with tissue and species-specific transcriptomic data, the following steps were not obvious, and so coexpression analysis allowed us to search for new candidates without prior knowledge of which enzyme families to search.

Throughout the process of characterizing various steps in the pathway, not every intermediate product was identified. Often it can be difficult to differentiate “actual” intermediates in terms of whether the observed products are relevant to the pathway or simply a result of an incomplete reconstruction or a heterologous host's interference of the native pathway. In the process of discovering the forskolin pathway, for example, coexpression of an incomplete set of genes in N. benthamiana led to an accumulation of many side products that did not occur once the entire pathway was reconstructed (five P450s acting on a single diterpene scaffold and at least sixteen total products)⁴⁰. A similar example can be seen with accumulation of precursors and side products for the scopolamine pathway in A. belladonna following virus-induced gene silencing of various pathway steps³. We identified the activity of the two TPSs and confirmed our predicted activity of two P450s, but following this confirmation, we decided to test enzymes in different combinations to identify new steps in case the side products seen were similar artifacts.

The presence of a minor product forming upon coexpression with AlaDC was expected based on the presence of aldehydes in our intermediates, however the amount of product that would form was uncertain. We proposed that ethylamine was the source of nitrogen in this pathway, however if that is the case, it is likely enzyme-catalyzed based on the poor conversion resulting from spontaneous condensation. It is more likely, however, that it follows a different mechanism than is proposed, as the product of SangRed converts nearly all of the products of CYP701A127 and CYP71FH1 to a single product which is likely an isomer of this spontaneous condensation based on an identical exact mass but differing retention time. The substrates and mechanism of SangRed is still unknown, and difficult to predict given its low degree of homology to other characterized enzymes.

### REFERENCES

(1) Galanie, S.; Thodey, K.; Trenchard, I. J.; Filsinger Interrante, M.; Smolke, C. D. Complete Biosynthesis of Opioids in Yeast. Science 2015, 349 (6252), 1095-1100. see website doi.org/10.1126/science.aac9373.
(2) Nett, R. S.; Lau, W.; Sattely, E. S. Discovery and Engineering of Colchicine Alkaloid Biosynthesis. Nature 2020, 584 (7819), 148-153. see website doi.org/10.1038/s41586-020-2546-8.
(3) Bedewitz, M. A.; Jones, A. D.; D'Auria, J. C.; Barry, C. S. Tropinone Synthesis via an Atypical Polyketide Synthase and P450-Mediated Cyclization. Nat Commun 2018, 9, 5281. see website doi.org/10.1038/s41467-018-07671-3.
(4) Wrenbeck, E. E.; Bedewitz, M. A.; Klesmith, J. R.; Noshin, S.; Barry, C. S.; Whitehead, T. A. An Automated Data-Driven Pipeline for Improving Heterologous Enzyme Expression. ACS Synth. Biol. 2019, 8 (3), 474-481. see website doi.org/10.1021/acssynbio.8b00486.
(5) Biosynthesis of medicinal tropane alkaloids in yeast|Nature. see website www.nature.com/articles/s41586-020-2650-9 (accessed 2021-04-15).

(6) Pan, Q.; Mustafa, N. R.; Tang, K.; Choi, Y. H.; Verpoorte, R. Monoterpenoid Indole Alkaloids Biosynthesis and Its Regulation in Catharanthus Roseus: A Literature Review from Genes to Metabolites. Phytochem Rev 2016, 15 (2), 221-250. see website doi.org/10.1007/s11101-015-9406-4.
(7) Caputi, L.; Franke, J.; Farrow, S. C.; Chung, K.; Payne, R. M. E.; Nguyen, T.-D.; Dang, T.-T. T.; Soares Teto Carqueijeiro, I.; Koudounas, K.; Duge de Bernonville, T.; Ameyaw, B.; Jones, D. M.; Vieira, I. J. C.; Courdavault, V.; O'Connor, S. E. Missing Enzymes in the Biosynthesis of the Anticancer Drug Vinblastine in Madagascar Periwinkle. Science 2018, 360 (6394), 1235-1239. see website doi.org/10.1126/science.aat4100.
(8) Qu, Y.; Safonova, O.; De Luca, V. Completion of the Canonical Pathway for Assembly of Anticancer Drugs Vincristine/Vinblastine in Catharanthus Roseus. The Plant Journal 2019, 97 (2), 257-266. see website doi.org/10.1111/tpj.14111.
(9) Lichman, B. R. The Scaffold-Forming Steps of Plant Alkaloid Biosynthesis. Nat. Prod. Rep. 2021, 38 (1), 103-129. see website doi.org/10.1039/DONP00031K.
(10) Oneto, J. F. The Alkaloids of Species of Garrya. I. Isolation of Alkaloids**University of California, College of Pharmacy, San Francisco. Journal of the American Pharmaceutical Association (Scientific ed.) 1946, 35 (7), 204-207. see website doi.org/10.1002/jps.3030350703.
(11) Ma, Y.; Mao, X.-Y.; Huang, L.-J.; Fan, Y.-M.; Gu, W.; Yan, C.; Huang, T.; Zhang, J.-X.; Yuan, C.-M.; Hao, X.-J. Diterpene Alkaloids and Diterpenes from Spiraea Japonica and Their Anti-Tobacco Mosaic Virus Activity. Fitoterapia 2016, 109, 8-13. see website doi.org/10.1016/j.fitote.2015.11.019.
(12) Hart, N.; Johns, S.; Lamberton, J.; Suares, H.; Willing, R. New Alkaloids of the Ent-Kaurene Type From Anopterus Species (Escalloniaceae). I. The Structure and Reactions of Anopterine. Aust. J. Chem. 1976, 29 (6), 1295-1318. see website doi.org/10.1071/ch9761295.
(13) Yin, T.; Cal, L.; Ding, Z. An Overview of the Chemical Constituents from the Genus Delphinium Reported in the Last Four Decades. RSC Advances 2020, 10 (23), 13669-13686. see website doi.org/10.1039/DORA00813C.
(14) Nyirimigabo, E.; Xu, Y.; Li, Y.; Wang, Y.; Agyemang, K.; Zhang, Y. A Review on Phytochemistry, Pharmacology and Toxicology Studies of Aconitum. J Pharm Pharmacol 2015, 67 (1), 1-19. see website doi.org/10.1111/jphp.12310.
(15) Csupor, D.; Wenzig, E. M.; Zupko, I.; Wolkart, K.; Hohmann, J.; Bauer, R. Qualitative and Quantitative Analysis of Aconitine-Type and Lipo-Alkaloids of Aconitum Carmichaelii Roots. Journal of Chromatography A 2009, 1216 (11), 2079-2086. see website doi.org/10.1016/j.chroma.2008.10.082.
(16) Zhou, G.; Tang, L.; Zhou, X.; Wang, T.; Kou, Z.; Wang, Z. A Review on Phytochemistry and Pharmacological Activities of the Processed Lateral Root of Aconitum Carmichaelii Debeaux. J Ethnopharmacol 2015, 160, 173-193. see website doi.org/10.1016/j.jep.2014.11.043.
(17) Liu, X.-Y.; Wang, F.-P.; Qin, Y. Synthesis of Three-Dimensionally Fascinating Diterpenoid Alkaloids and Related Diterpenes. Acc. Chem. Res. 2021, 54 (1), 22-34. see website doi.org/10.1021/acs.accounts.0c00720.
(18) Gong, J.; Chen, H.; Liu, X.-Y.; Wang, Z.-X.; Nie, W.; Qin, Y. Total Synthesis of Atropurpuran. Nat Commun 2016, 7 (1), 12183. see website doi.org/10.1038/ncomms12183.
(19) Owens, K. R.; McCowen, S. V.; Blackford, K. A.; Ueno, S.; Hirooka, Y.; Weber, M.; Sarpong, R. Total Synthesis of the Diterpenoid Alkaloid Arcutinidine Using a Strategy Inspired by Chemical Network Analysis. J. Am. Chem. Soc. 2019, 141 (35), 13713-13717. see website doi.org/10.1021/jacs.9b05815.
(20) Pang, L.; Liu, C.-Y.; Gong, G.-H.; Quan, Z.-S. Synthesis, in Vitro and in Vivo Biological Evaluation of Novel Lappaconitine Derivatives as Potential Anti-Inflammatory Agents. Acta Pharm Sin B 2020, 10 (4), 628-645. see website doi.org/10.1016/j.apsb.2019.09.002.
(21) Cherney, E. C.; Baran, P. S. Terpenoid-Alkaloids: Their Biosynthetic Twist of Fate and Total Synthesis. Isr J Chem 2011, 51 (3-4), 391-405. see website doi.org/10.1002/ijch.201100005.
(22) Li, Y.-G.; Mou, F.-J.; Li, K.-Z. De Novo RNA Sequencing and Analysis Reveal the Putative Genes Involved in Diterpenoid Biosynthesis in Aconitum Vilmorinianum Roots. 3 Biotech 2021, 11 (2), 96. see website doi.org/10.1007/s13205-021-02646-6.
(23) Pal, T.; Malhotra, N.; Chanumolu, S. K.; Chauhan, R. S. Next-Generation Sequencing (NGS) Transcriptomes Reveal Association of Multiple Genes and Pathways Contributing to Secondary Metabolites Accumulation in Tuberous Roots of Aconitum Heterophyllum Wall. Planta 2015, 242 (1), 239-258. see website doi.org/10.1007/s00425-015-2304-6.
(24) Rai, M.; Rai, A.; Kawano, N.; Yoshimatsu, K.; Takahashi, H.; Suzuki, H.; Kawahara, N.; Saito, K.; Yamazaki, M. De Novo RNA Sequencing and Expression Analysis of Aconitum Carmichaelii to Analyze Key Genes Involved in the Biosynthesis of Diterpene Alkaloids. Molecules 2017, 22 (12). see website doi.org/10.3390/molecu1es22122155.
(25) Yang, Y.; Hu, P.; Zhou, X.; Wu, P.; Si, X.; Lu, B.; Zhu, Y.; Xia, Y. Transcriptome Analysis of Aconitum Carmichaelii and Exploration of the Salsolinol Biosynthetic Pathway. Fitoterapia 2020, 140, 104412. see website doi.org/10.1016/j.fitote.2019.104412.
(26) Zhao, D.; Shen, Y.; Shi, Y.; Shi, X.; Qiao, Q.; Zi, S.; Zhao, E.; Yu, D.; Kennelly, E. J. Probing the Transcriptome of Aconitum Carmichaelii Reveals the Candidate Genes Associated with the Biosynthesis of the Toxic Aconitine-Type C19-Diterpenoid Alkaloids. Phytochemistry 2018, 152, 113-124. see website doi.org/10.1016/j.phytochem.2018.04.022.
(27) Mao, L.; Jin, B.; Chen, L.; Tian, M.; Ma, R.; Yin, B.; Zhang, H.; Guo, J.; Tang, J.; Chen, T.; Lai, C.; Cui, G.; Huang, L. Functional Identification of the Terpene Synthase Family Involved in Diterpenoid Alkaloids Biosynthesis in Aconitum Carmichaelii. Acta Pharmaceutica Sinica B 2021. see website doi.org/10.1016/j.apsb.2021.04.008.
(28) Miller, G. P.; Bhat, W. W.; Lanier, E. R.; Johnson, S. R.; Mathieu, D. T.; Hamberger, B. The Biosynthesis of the Anti-Microbial Diterpenoid Leubethanol in Leucophyllum Frutescens Proceeds via an All-Cis Prenyl Intermediate. The Plant Journal 2020, 104 (3), 693-705. see website doi.org/10.1111/tpj.14957.
(29) Jin, B.; Cui, G.; Guo, J.; Tang, J.; Duan, L.; Lin, H.; Shen, Y.; Chen, T.; Zhang, H.; Huang, L. Functional Diversification of Kaurene Synthase-Like Genes in Isodon Rubescens. Plant Physiology 2017, 174 (2), 943-955. see website doi.org/10.1104/pp. 17.00202.
(30) Grennan, A. K. Gibberellin Metabolism Enzymes in Rice. Plant Physiology 2006, 141 (2), 524-526. see website doi.org/10.1104/pp. 104.900192.
(31) Kong, H.; Zhang, Y.; Hong, Y.; Barker, M. S. Multilocus Phylogenetic Reconstruction Informing Polyploid Relationships of Aconitum Subgenus Lycoctonum (Ranunculaceae) in China. Plant Syst Evol 2017, 303 (6), 727-744. see website doi.org/10.1007/s00606-017-1406-y.
(32) Park, S.; An, B.; Park, S. Recurrent Gene Duplication in the Angiosperm Tribe Delphinieae (Ranunculaceae) Inferred from Intracellular Gene Transfer Events and Heteroplasmic Mutations in the Plastid MatK Gene. Sci Rep 2020, 10 (1), 2720. see website doi.org/10.1038/s41598-020-59547-6.
(33) Salvado, P.; Aymerich Boixader, P.; Parera, J.; Vila Bonfill, A.; Martin, M.; Quelennec, C.; Lewin, J.-M.; Delorme-Hinoux, V.; Bertrand, J. A. M. Little Hope for the Polyploid Endemic Pyrenean Larkspur (Delphinium Montanum): Evidences from Population Genomics and Ecological Niche Modeling. Ecology and Evolution 2022, 12 (3) e8711. see website doi.org/10.1002/ece3.8711.
(34) Xu, J.-B.; Li, Y.-Z.; Huang, S.; Chen, L.; Luo, Y.-Y.; Gao, F.; Zhou, X.-L. Diterpenoid Alkaloids from the Whole Herb of Delphinium Grandiflorum L. Phytochemistry 2021, 190, 112866. see website doi.org/10.1016/j.phytochem.2021.112866.
(35) Li, Y.; Gao, F.; Zhang, J.-F.; Zhou, X.-L. Four New Diterpenoid Alkaloids from the Roots of Aconitum Carmichaelii. Chem. Biodivers. 2018, 15 (7), e1800147. see website doi.org/10.1002/cbdv.201800147.
(36) Yamashita, H.; Takeda, K.; Haraguchi, M.; Abe, Y.; Kuwahara, N.; Suzuki, S.; Terui, A.; Masaka, T.; Munakata, N.; Uchida, M.; Nunokawa, M.; Kaneda, K.; Goto, M.; Lee, K.-H.; Wada, K. Four New Diterpenoid Alkaloids from Aconitum Japonicum Subsp. Subcuneatum. J Nat Med 2018, 72 (1), 230-237. see website doi.org/10.1007/s11418-017-1139-9.
(37) Yin, T.-P.; Cal, L.; Fang, H.-X.; Fang, Y.-S.; Li, Z.-J.; Ding, Z.-T. Diterpenoid Alkaloids from Aconitum Vilmorinianum. Phytochemistry 2015, 116, 314-319. see website doi.org/10.1016/j.phytochem.2015.05.002.
(38) Andersen-Ranberg, J.; Kongstad, K. T.; Nielsen, M. T.; Jensen, N. B.; Pateraki, I.; Bach, S. S.; Hamberger, B.; Zerbe, P.; Staerk, D.; Bohlmann, J.; Møller, B. L.; Hamberger, B. Expanding the Landscape of Diterpene Structural Diversity through Stereochemically Controlled Combinatorial Biosynthesis. Angewandte Chemie International Edition 2016, 55 (6), 2142-2146. see website doi.org/10.1002/anie.201510650.
(39) Nelson, D.; Werck-Reichhart, D. A P450-Centric View of Plant Evolution. The Plant Journal 2011, 66 (1), 194-211. see website doi.org/10.1111/j.1365-313X.2011.04529.x.
(40) Pateraki, I.; Andersen-Ranberg, J.; Jensen, N. B.; Wubshet, S. G.; Heskes, A. M.; Forman, V.; Hallstrom, B.; Hamberger, B.; Motawia, M. S.; Olsen, C. E.; Staerk, D.; Hansen, J.; Møller, B. L.; Hamberger, B. Total Biosynthesis of the Cyclic AMP Booster Forskolin from Coleus Forskohlii. eLife 2017, 6, e23001. see website doi.org/10.7554/eLife.23001.
(41) Bal, P.; Wang, L.; Wei, K.; Ruan, L.; Wu, L.; He, M.; Ni, D.; Cheng, H. Biochemical Characterization of Specific Alanine Decarboxylase (AlaDC) and Its Ancestral Enzyme Serine Decarboxylase (SDC) in Tea Plants (Camellia Sinensis). BMC Biotechnology 2021, 21 (1), 17. see website doi.org/10.1186/s12896-021-00674-x.
(42) Zhao, P.-J.; Gao, S.; Fan, L.-M.; Nie, J.-L.; He, H.-P.; Zeng, Y.; Shen, Y.-M.; Hao, X.-J. Approach to the Biosynthesis of Atisine-Type Diterpenoid Alkaloids. J. Nat. Prod. 2009, 72 (4), 645-649. see website doi.org/10.1021/np800657j.
(43) Wisecaver, J. H.; Borowsky, A. T.; Tzin, V.; Jander, G.; Kliebenstein, D. J.; Rokas, A. A Global Coexpression Network Approach for Connecting Genes to Specialized Metabolic Pathways in Plants. Plant Cell 2017, 29 (5), 944-959. see website doi.org/10.1105/tpc.17.00009.
(44) Bosch i Daniel, M.; Simon Pallisé, J.; López i Pujol, J.; Blanché i Vergés, C. DCDB: An Updated on-Line Database of Chromosome Numbers of Tribe Delphinieae (Ranunculaceae). 2016.
(45) Morrone, D.; Chen, X.; Coates, R. M.; Peters, R. J. Characterization of the Kaurene Oxidase CYP701A3, a Multifunctional Cytochrome P450 from Gibberellin Biosynthesis. Biochemical Journal 2010, 431 (3), 337-347. see website doi.org/10.1042/BJ20100597.
(46) Prisic, S.; Xu, M.; Wilderman, P. R.; Peters, R. J. Rice Contains Two Disparate Ent-Copalyl Diphosphate Synthases with Distinct Metabolic Functions. Plant Physiol 2004, 136 (4), 4228-4236. see website doi.org/10.1104/pp. 104.050567.
(47) Harris, L. J.; Saparno, A.; Johnston, A.; Prisic, S.; Xu, M.; Allard, S.; Kathiresan, A.; Ouellet, T.; Peters, R. J. The Maize An2 Gene Is Induced by Fusarium Attack and Encodesan Ent-Copalyl Diphosphate Synthase. Plant Mol Biol 2005, 59 (6), 881-894. see website doi.org/10.1007/s11103-005-1674-8.
(48) Kumar, S.; Stecher, G.; Suleski, M.; Hedges, S. B. TimeTree: A Resource for Timelines, Timetrees, and Divergence Times. Mol Biol Evol 2017, 34 (7), 1812-1819. see website doi.org/10.1093/molbev/msx116.
(49) Minami, H.; Dubouzet, E.; Iwasa, K.; Sato, F. Functional Analysis of Norcoclaurine Synthase in Coptis Japonica. J Biol Chem 2007, 282 (9), 6274-6282. see website doi.org/10.1074/jbc.M608933200.
(50) Li, W.; Godzik, A. Cd-Hit: A Fast Program for Clustering and Comparing Large Sets of Protein or Nucleotide Sequences. Bioinformatics 2006, 22 (13), 1658-1659. see website doi.org/10.1093/bioinformatics/bt1158.
(51) Fu, L.; Niu, B.; Zhu, Z.; Wu, S.; Li, W. CD-HIT: Accelerated for Clustering the next-Generation Sequencing Data. Bioinformatics 2012, 28 (23), 3150-3152. see website doi.org/10.1093/bioinformatics/bts565.
(52) Shannon, P.; Markiel, A.; Ozier, O.; Baliga, N. S.; Wang, J. T.; Ramage, D.; Amin, N.; Schwikowski, B.; Ideker, T. Cytoscape: A Software Environment for Integrated Models of Biomolecular Interaction Networks. Genome Res. 2003, 13 (11), 2498-2504. doi.org/10.1101/gr.1239303.
(53) Johnson, S. R.; Bhat, W. W.; Bibik, J.; Turmo, A.; Hamberger, B.; Hamberger, B. A Database-Driven Approach Identifies Additional Diterpene Synthase Activities in the Mint Family (Lamiaceae). J Biol Chem 2019, 294 (4), 1349-1362. see website doi.org/10.1074/jbc.RA118.006025.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various features of the invention according to the foregoing description provided in the specification and figures.

### Statements:

1. An expression system comprising at least one expression cassette having a heterologous promoter operably linked to a nucleic acid segment encoding an enzyme with at least 90% sequence identity to amino acid SEQ ID NO: 1, 3, 5, 7, 9, 11, or 13.
2. The expression system of statement 1, wherein at least one expression cassette is within at least one expression vector.
3. The expression system of statement 1 or 2, wherein the expression system comprises two, or three, or four, or five expression cassettes or expression vectors, each expression cassette encoding a separate enzyme.
4. The expression system of statement 1, 2 or 3, wherein the expression system further comprises one or more expression cassettes having a promoter operably linked to a nucleic acid segment encoding an enzyme that can synthesize isopentenyl diphosphate (IPP), dimethylallyl diphosphate (DMAPP), or geranylgeranyl diphosphate (GGPP), or a combination thereof.
5. The expression system of statement 1-3 or 4, wherein the expression system has at least one expression cassette having a constitutive promoter.
6. The expression system of statement 1-3 or 4, wherein the expression system has at least one expression cassette having an inducible promoter.
7. The expression system of statement 1-5 or 6, wherein the expression system has at least one expression cassette having a CaMV 35S promoter, CaMV 19S promoter, nos promoter, Adh1 promoter, sucrose synthase promoter, α-tubulin promoter, ubiquitin promoter, actin promoter, cab promoter, PEPCase promoter, R gene complex promoter, CYP71D16 trichome-specific promoter, CBTS (cembratrienol synthase) promotor, Z10 promoter from a 10 kD zein protein gene, Z27 promoter from a 27 kD zein protein gene, plastid rRNA-operon (rrn) promoter, light inducible pea rbcS gene, RUBISCO-SSU light-inducible promoter (SSU) from tobacco, or rice actin promoter.
8. A host cell comprising the expression system of statement 1-6 or 7, which is heterologous to the host cell.
9. The host cell of statement 8, which is a plant cell, an algae cell, a fungal cell, a bacterial cell, or an insect cell.
10. The host cell of statement 8 or 9, which is a Nicotiana benthamiana, Nicotiana tabacum, Nicotiana rustica, Nicotiana excelsior, Nicotiana excelsiana, Escherichia coli, Clostridium ljungdahlii, Clostridium autoethanogenum, Clostridium kluyveri, Corynebacterium glutamicum, Cupriavidus necator, Cupriavidus metallidurans; Pseudomonas fluorescens, Pseudomonas putida, Pseudomonas oleavorans; Delftia acidovorans, Bacillus subtilis, Lactobacillus delbrueckii, Lactococcus lactis, Aspergillus niger, Saccharomyces cerevisiae, Candida tropicalis, Candida albicans, Candida cloacae, Candida guillermondii, Candida intermedia, Candida maltosa, Candida parapsilosis, Candida zeylenoides, Pichia pastoris, Yarrowia lipolytica, Issathenkia orientalis, Debaryomyces hansenii, Arxula adenoinivorans, Kluyveromyces lactis, or Exophiala, Mucor, Trichoderma, Cladosporium, Phanerochaete, Cladophialophora, Paecilomyces, Scedosporium, or Ophiostoma cell.
11. The host cell of statement 8, 9 or 10, which is a Nicotiana benthamiana.
12. A method of synthesizing a diterpenoid alkaloid comprising incubating a host cell that has the expression system of any of statements 1-7.
13. A method for synthesizing a diterpenoid alkaloid comprising incubating a host cell comprising a heterologous expression system that includes at least one expression cassette having a heterologous promoter operably linked to a nucleic acid segment encoding an enzyme with at least 90% sequence identity to SEQ ID NO:1, 3, 5, 7, 9, 11, or 13.
14. A method for synthesizing a diterpenoid alkaloid comprising incubating a terpene precursor with an enzyme with at least 90% sequence identity to SEQ ID NO: 1, 3, 5, 7, 9, 11, or 13.
15. The method of statement 13 or 14, wherein the diterpenoid alkaloid comprises a19 or 20 carbon ring structure containing a nitrogen.
16. The method of statement 13, 14 or 15, wherein the diterpenoid alkaloid has a tetracyclic ring structure.
17. The method of statement 16, wherein each of the rings in the tetracyclic ring structure has ring atoms.
18. The method of statement 16 or 17, wherein each of the rings in the tetracyclic ring structure has 6 ring atoms.
19. The method of statement 16, 17 or 18, wherein one ring in the tetracyclic ring structure has 6 ring atoms, a second ring in the tetracyclic ring structure has 7 ring atoms, a third ring in the tetracyclic ring structure has 5 ring atoms, and a fourth ring in the tetracyclic ring structure has 6 ring atoms.
20. The method of any one of statements 16-19, wherein the diterpenoid alkaloid is aconitine or a C20 hetidine-type diterpenoid alkaloid
21. The method of any one of statements 16-20, wherein the diterpenoid alkaloid comprises any one of the following compounds:

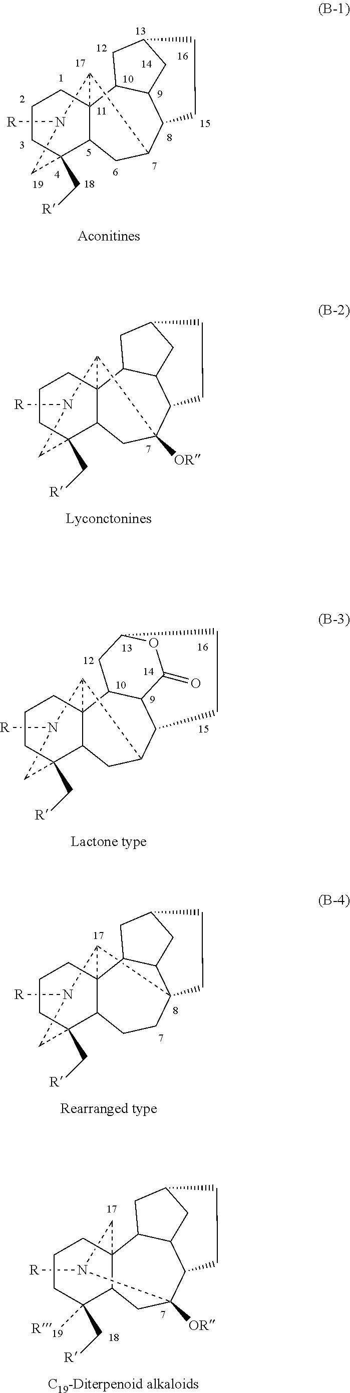

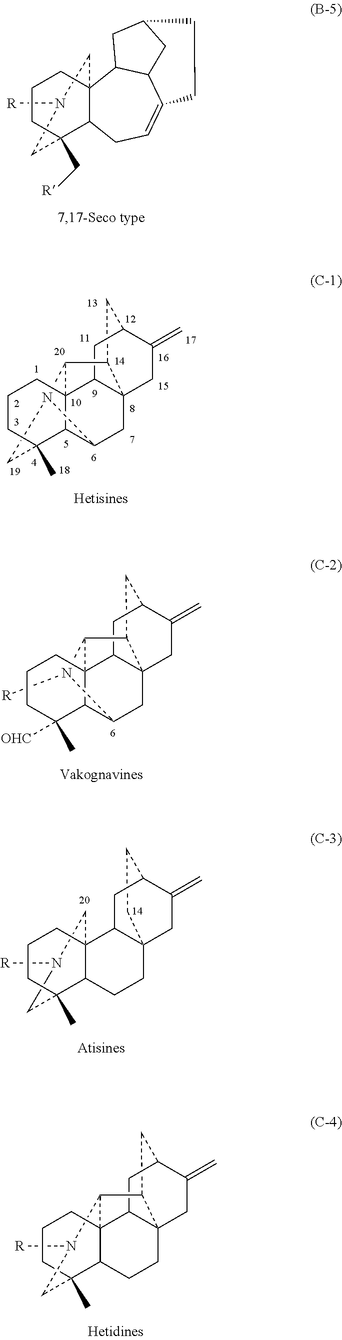

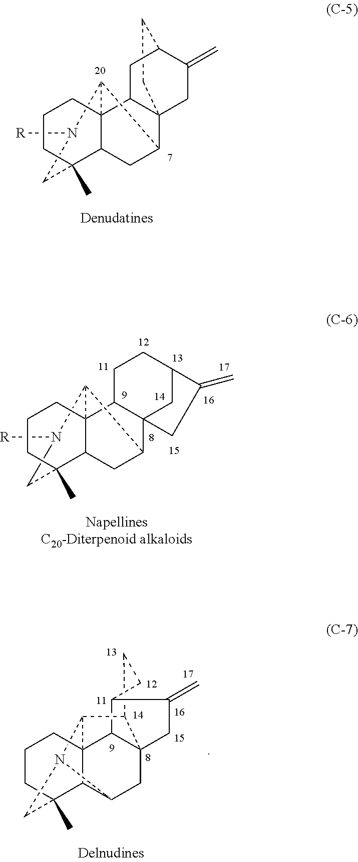

22. The method of any one of statements 16-21, wherein the terpene precursor is geranylgeranyl diphosphate (GGPP).

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. An expression system comprising at least one expression cassette having a heterologous promoter operably linked to a nucleic acid segment encoding an enzyme with at least 95% sequence identity to an amino acid sequence of SEQ ID NO: 1, 3, 5, 7, 9, 11, or 13.

2. The expression system of claim 1, wherein the expression system comprises at least two, or three, or four, or five expression cassettes or expression vectors, each expression cassette encoding a separate enzyme.

3. The expression system of claim 1, wherein the expression system further comprises one or more expression cassettes having a promoter operably linked to a nucleic acid segment encoding an enzyme that can synthesize isopentenyl diphosphate (IPP), dimethylallyl diphosphate (DMAPP), geranylgeranyl diphosphate (GGPP), or a combination thereof.

4. The expression system of claim 1, wherein the expression system has at least one expression cassette having a constitutive promoter.

5. The expression system of claim 1, wherein the expression system has at least one expression cassette having an inducible promoter.

6. The expression system of claim 1, wherein the expression system has at least one expression cassette having a CaMV 35S promoter, CaMV 19S promoter, nos promoter, Adh1 promoter, sucrose synthase promoter, α-tubulin promoter, ubiquitin promoter, actin promoter, cab promoter, PEPCase promoter, R gene complex promoter, CYP71D16 trichome-specific promoter, CBTS (cembratrienol synthase) promotor, Z10 promoter from a 10 kD zein protein gene, Z27 promoter from a 27 kD zein protein gene, plastid rRNA-operon (rrn) promoter, light inducible pea rbcS gene, RUBISCO-SSU light-inducible promoter (SSU) from tobacco, or rice actin promoter.

7. A host cell comprising the expression system of claim 1, which is heterologous to the host cell.

8. A host cell comprising the expression system of claim 1, wherein the expression system further comprises one or more expression cassettes having a promoter operably linked to a nucleic acid segment encoding an enzyme that can synthesize isopentenyl diphosphate (IPP), dimethylallyl diphosphate (DMAPP), geranylgeranyl diphosphate (GGPP), or a combination thereof.

9. The host cell of claim 7, which is a plant cell, an algae cell, a fungal cell, a bacterial cell, or an insect cell.

10. The host cell of claim 7, which is a Nicotiana benthamiana, Nicotiana tabacum, Nicotiana rustica, Nicotiana excelsior, Nicotiana excelsiana, Escherichia coli, Clostridium ljungdahlii, Clostridium autoethanogenum, Clostridium kluyveri, Corynebacterium glutamicum, Cupriavidus necator, Cupriavidus metallidurans; Pseudomonas fluorescens, Pseudomonas putida, Pseudomonas oleavorans; Delftia acidovorans, Bacillus subtilis, Lactobacillus delbrueckii, Lactococcus lactis, Aspergillus niger, Saccharomyces cerevisiae, Candida tropicalis, Candida albicans, Candida cloacae, Candida guillermondii, Candida intermedia, Candida maltosa, Candida parapsilosis, Candida zeylenoides, Pichia pastoris, Yarrowia lipolytica, Issathenkia orientalis, Debaryomyces hansenii, Arxula adenoinivorans, Kluyveromyces lactis, or Exophiala, Mucor, Trichoderma, Cladosporium, Phanerochaete, Cladophialophora, Paecilomyces, Scedosporium, or Ophiostoma cell.

11. The host cell of claim 7, which is a Nicotiana benthamiana.

12. A method for synthesizing a diterpenoid alkaloid comprising incubating a host cell comprising a heterologous expression system that includes at least one expression cassette having a heterologous promoter operably linked to a nucleic acid segment encoding an enzyme with at least 90% sequence identity to SEQ ID NO:1, 3, 5, 7, 9, 11, or 13.

13. The method of claim 12, wherein the diterpenoid alkaloid comprises a 19 or 20 carbon ring structure containing a nitrogen.

14. The method of claim 12, wherein the diterpenoid alkaloid has a tetracyclic ring structure.

15. A method for synthesizing a diterpenoid alkaloid comprising incubating a terpene precursor with an enzyme with at least 90% sequence identity to SEQ ID NO: 1, 3, 5, 7, 9, 11, or 13.

16. The method of claim 15, wherein the diterpenoid alkaloid comprises a 19 or 20 carbon ring structure containing a nitrogen.

17. The method of claim 15, wherein the diterpenoid alkaloid has a tetracyclic ring structure.

18. The method of claim 15, wherein each of the rings in the tetracyclic ring structure has ring atoms.

19. The method of claim 15, wherein each of the rings in the tetracyclic ring structure has 6 ring atoms.

20. The method of claim 15, wherein one ring in the tetracyclic ring structure has 6 atoms, a second ring in the tetracyclic ring structure has 7 atoms, a third ring in the tetracyclic ring structure has 5 atoms, and a fourth ring in the tetracyclic ring structure has 6 atoms.

21. The method of claim 15, wherein the diterpenoid alkaloid is aconitine or a C20 hetidine-type diterpenoid alkaloid.

22. The method of claim 15, wherein the diterpenoid alkaloid comprises any one of the following compounds:

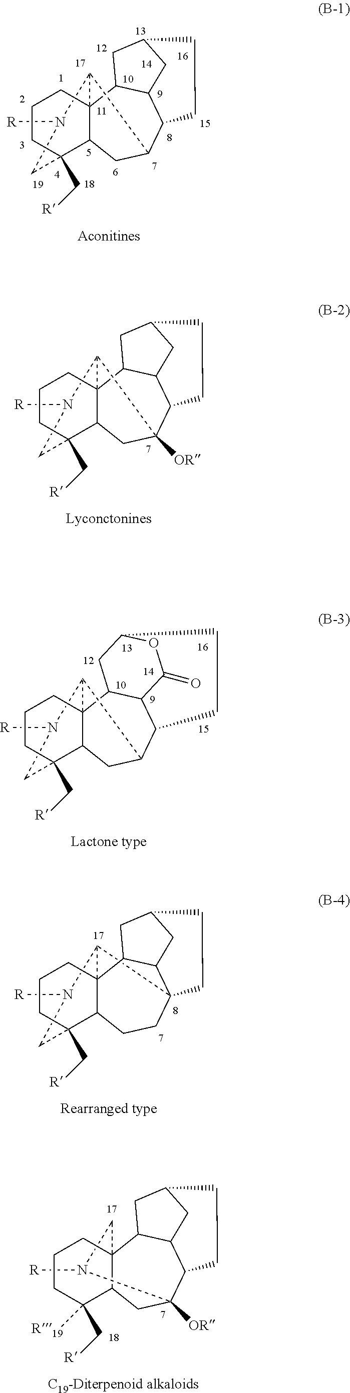

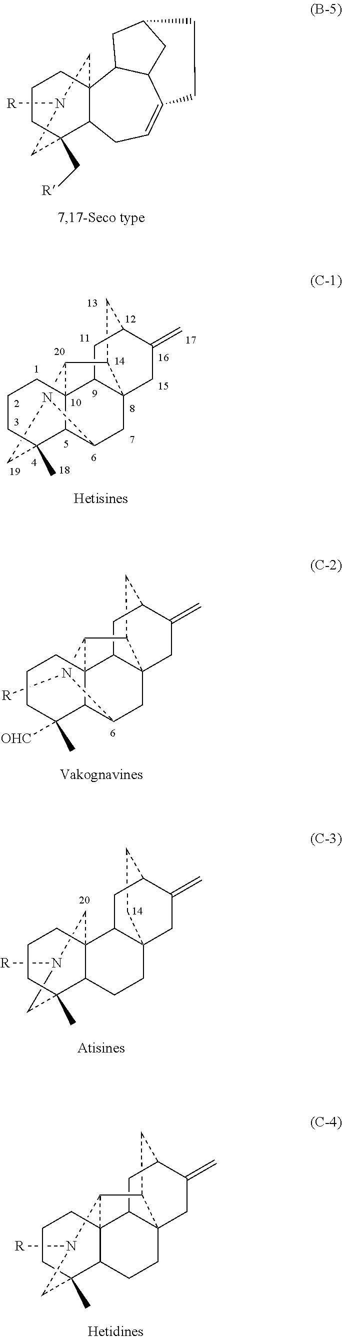

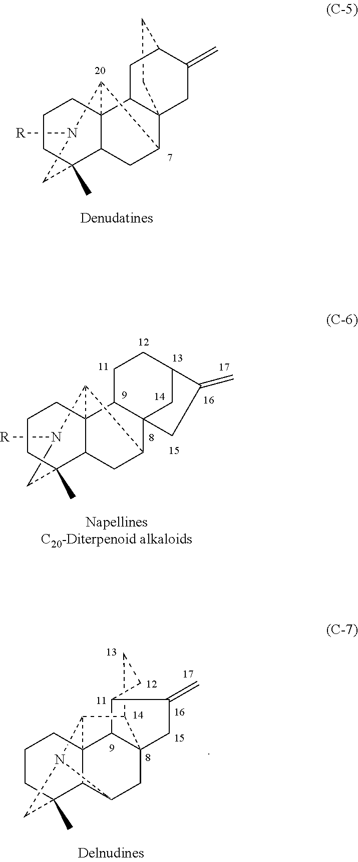

23. The method of claim 15, wherein the terpene precursor is geranylgeranyl diphosphate (GGPP).


SEQUENCE LISTING (14 sequences)

SEQ ID NO: 1 - protein, 794 aa, Delphinium grandiflorum
MASLSLHSASSHLSASPAEVSPPLFSSGFAHSLPVKNKRDDGHNSRCSATSKHDGQVYKEVTKQDTIRKWQEITNQDSKNGAVKVDDINKLAEWIGDIKNMLRSMDDGEISVSAYDTAWVALVENIHGFYGPQFPSSVEWIVNNQLGDGSWGDEPIFSAHDRILNTLGCVVALKTWSIHPEKCEKGLSYIRQNISRLDDESTEHMPIGFEIAFPSLIEMARKLNLDIPYDSAAVLAIYAQKDIKLMKIPMEKAHKWPTTLLHSLEGMDGLDWDKLMKLQSSNGSFLFSPASTAFALMNTKDEKCLEYLKKPVEKFNGGVPNVYPVDLFEHIWVVDRLERLGVSRYFEAEIKDCIDYVAKYWTKSGIAWARNSTVCDIDDTAMGFRLLRLHGYNVSPDVFKNFQNGDEFVCFAGQSNQAVTGMYNLYRAAQVAFPGETILEDCKKFSYKFLRNKQATNQLLDKWIITKDLPGEVGYALDFPWYANLPRIETRLYLEQYGGDEDVWIGKTLYRMSYVNNGTYLNAAKLDFNNCQAVHHVEWDNIQKWYLECNLAEFGVTDARLLQTYFVATASIFEPERSSERLAWTKIALLLESILSHFKDETKEHRKAFIVDFIENKVVSRKLNYSTGKASNLVHTLVGTLQDIAITNGSGIQNALLDTFEKWLFTWEIRFSSKEVAGLLANMINICSGNEVSDEVSSNPEYRSLVDLTNKICFQLGQASKVGINGTRVNGLEIPSVELDMEELVKIVVRKDNGIDSKVKQTFLEVVKSFFYVSQCPKEVMERHIEEVLFNRVA

SEQ ID NO: 2 - DNA, 2385 bp, Delphinium grandiflorum
  source 1..2385: mol_type = other DNA
atggcctctctctccctccactctgcttcttcccacctctcagcatcacctgcagaggtatcacctccactgttttcatcaggatttgctcattcacttcctgttaagaataaacgcgatgatggtcacaactcaagatgctctgcaacatcgaaacatgatggtcaagtatataaagaggttacgaagcaggatacgataagaaaatggcaagaaattacaaaccaagatagcaagaacggcgcggttaaggttgatgatatcaacaagctagcagagtggattggagacataaaaaatatgctgcgttctatggacgatggggagataagcgtctcggcctatgacacggcttgggttgctctggtcgaaaacattcatggcttttatggccctcagtttccgtcgagtgttgaatggatcgttaataatcagctaggtgatggttcctggggcgatgagcctattttctctgcacatgatcggatactaaatacattgggctgtgtggttgcgttaaaaacatggagcattcatcccgagaaatgcgagaagggattgtcgtatatccgtcagaacatcagcaggctggatgatgaaagtactgaacacatgcctatagggtttgagatcgcctttccttctcttatcgaaatggcacggaagttaaacttggatatcccctatgactcggctgcagtgctcgcaatatacgcccaaaaggatataaagctcatgaagataccgatggagaaggcacataaatggcccactacgctacttcacagtttggaaggcatggatggattggattgggataaacttatgaagttgcaaagctcaaatggctccttcttgttctctccagcatcgacggccttcgcccttatgaacactaaagatgaaaagtgtcttgaatatctcaagaaaccggttgaaaaattcaatggtggagtcccgaatgtctatcctgtagacttgtttgaacatatttgggtggttgatcgtttggaacgtcttggagtttcacgctacttcgaggcagaaatcaaagattgcatcgactatgtagctaaatattggactaaatctgggatagcttgggcgagaaactcgactgtttgtgacatagatgacacggccatggggttcaggcttctacgcctacatggatacaacgtctcccctgatgtgtttaagaattttcaaaacggcgatgagtttgtttgttttgctggacaatcaaaccaggccgttacagggatgtacaatctttatagggctgctcaggtggccttccctggggagactatcctggaagattgcaagaaattttcctacaaatttcttcgcaataaacaagctaccaaccaacttttagataaatggatcataacaaaggatttgccaggggaggttgggtacgccctagattttccatggtatgcaaacctaccccgaatcgaaacacgcctttacttggaacaatatggtggtgatgaagacgtctggatagggaaaacgctttacaggatgtcgtatgttaacaatggcacatatcttaacgcggccaaactagacttcaataattgtcaagcagtccatcatgttgaatgggataatatccaaaagtggtaccttgagtgcaatctagctgagttcggagtgaccgatgcaagacttctacaaacttattttgtagctactgcaagcatatttgagcctgaaagatcgtctgagaggcttgcatggaccaagattgctttgctcctcgagtcaattttgtcacacttcaaagatgaaaccaaggaacaccgaaaggcgtttatcgtcgactttattgagaataaggttgtatcaaggaaattgaactactccactggcaaggcaagcaatcttgtgcatactcttgttgggaccttacaagatatcgcaataaccaatggaagcggcattcagaacgcactacttgatacttttgagaagtggttgtttacttgggaaatccggttttcttcaaaagaagtagcgggacttttggccaacatgataaacatatgcagtggaaatgaagtttctgatgaggtttcatccaatcctgaatatcgaagtcttgtcgacttgaccaataaaatctgcttccaacttggtcaggctagtaaggttgggataaacggcacacgagtgaatggcttggagataccatcggttgaactcgatatggaggagctagtgaagattgttgttaggaaggacaatggaatcgacagtaaggtcaagcagacgttcctcgaagttgtgaaaagcttcttctatgtctctcagtgtccaaaagaagtgatggagcgtcacatcgaagaagtcctcttcaaccgagtagcctaa

SEQ ID NO: 3 - protein, 801 aa, Delphinium grandiflorum
MYLSHPTKSPLVFPNPTTSSPRGSSSTSISAVSVDHGVKRLEKSENSLKISEATKEKISKIFTKVELSKSSYDTAWVAMVPSLDSSASPYFPECLNWILENQHTDGSWGLTQQHPLLLKDTLSSTLASILALKRWNVGEDHVNKGLHFISSNFASATDEKQRCPIGFDIIFPGMIERAQEIGVNFHLDPTSLNSILSKRDTELHRVSTSNSEGSKLYRAYFAEGLRKSQNWEEVMKYQRKNGSLFNSPSTTAVAAAHVQDPNCFKYLHSILEEFGNAVPTSYPLDIYTQLCMIDALEKLGISRHFKNEVGNVLDKTYSSWLTKDEEIFLDVSTSAMAFRILRVHGYDVSPDVLAQFGQEGFSNTLGGYLNDSGAVLEIYRASQIVLPNEVFLEEQKSWSSAYLKNELSKGSMHADRMHEWISKEVETALTYPYKPNLPRLEHRRTVEHYNVDNLRVLKSAYRPLGIDNKDLLHLAMEDFNICQSIYQNEFKELERWVKDNRIDKLKFARQKQVYTLFSSASTLFPPELSDARLSWAKFSILTTIIDDCYDLGGSRDELINLNQVFDKWDGVTAGDFISEPVEILYYAYKNTIDDLARKAFKYQHRDITKHLVENCVEMVKSMWIEAEWMEHNVVPSLEEYNENGYVSFALGPIVLTTLYFVGPQLSEEVVRSSEYHDLFRLMSTICRNLNDLRTVQKELSEGTINGVSILMIHDPEVKTEEDSVKKIREAIEICEKELIKLVLRRKDCVVPRACKELFWNMIRINNLFYASIDGYTSETQMMNEVKAVMRIPLTRPDLIEG

SEQ ID NO: 4 - DNA, 2406 bp, Delphinium grandiflorum
  source 1..2406: mol_type = other DNA
atgtatctctcccatccaaccaagtcgcctctcgtctttccgaacccaacaacatcatcgccgaggggatcctcctccacatccatctcagctgtttctgtggatcatggtgttaagaggttggaaaaatctgaaaattctcttaagatttccgaggcgaccaaggagaaaataagcaaaatcttcaccaaggttgagctttcgaaatcttcatacgacaccgcttgggttgcaatggtcccttctcttgactcctctgcatcgccctactttcccgaatgtctcaactggatcttggagaatcaacacacggacggctcatggggccttactcagcaacaccctttattgttaaaggacacgctgtcgtcgacattagcctctatacttgcactcaaaagatggaatgtcggcgaagaccatgttaacaagggtctccatttcattagttctaattttgcttccgccacagacgagaagcaacgttgtccaattgggtttgacatcatattccccggtatgatcgagcgtgctcaggagataggagtaaacttccatttagacccaacgagtttaaattctattcttagtaagagagacacggaattacatagggtatctacaagcaactcagagggaagcaaactctaccgagcctactttgcggagggactgaggaaatcgcaaaattgggaggaagtaatgaaatatcagagaaagaatggatcgttgtttaactctccttccaccactgcggtcgcggcggctcacgttcaagacccgaattgcttcaagtacttgcactcgatcttggaggaattcggcaatgcagtcccgactagttatccactagacatatacacccagctctgtatgattgacgctctagagaaactgggaatctcccgacacttcaagaatgaggtaggaaatgttttggataaaacctacagttcctggctgaccaaggatgaggaaatctttttagacgtttcaacatcggccatggcatttaggatattacgtgtacatggatacgacgtctccccagacgtactagctcaattcggccaagaaggtttctcaaatacacttggaggatacctaaacgactcaggggctgtccttgagatatatcgggcgtcccaaattgtgctccccaatgaggtatttctggaggaacaaaaatcttggtcaagtgcttatcttaagaatgaactatccaagggttcgatgcacgccgatagaatgcatgaatggattagcaaagaggtcgaaacggcgcttacctatccctacaaacccaatttgccgcgcttagagcacaggagaaccgtggaacattacaatgtcgataacttgagagttctgaaatcagcatataggcctcttggtattgacaacaaggatttactgcatttggcgatggaagattttaatatttgtcaatcgatatatcaaaatgaattcaaggagctcgagaggtgggtgaaagacaacaggatagataagctaaagttcgcaaggcaaaagcaggtgtacacgctcttctcttccgcatcaactctatttcctccagaattaagtgacgcgcgtctctcgtgggcaaagttcagtatcctcacaactataattgacgattgctacgatttaggcggctctagagacgaactaattaacctaaaccaagtgtttgacaagtgggatggagttacagccggtgacttcatttccgagccagttgaaatactatattatgcatacaaaaatacgattgatgatcttgcaagaaaggctttcaaatatcagcatcgggatatcacaaagcatttagtggagaactgtgttgaaatggttaagtctatgtggatcgaggcagagtggatggagcacaatgtagtaccatcactggaagaatacaatgaaaatggatacgtatcgtttgctctggggcctatagttcttacaactttatattttgttgggccccaactttccgaggaagtcgtaaggagttctgagtaccatgacctatttcgactcatgagcacaatatgtcgtaacctcaatgatcttcgaacagttcagaaggaactaagcgaagggacgataaacggtgtgtccattctgatgatacacgaccctgaagtcaagacggaggaagactcggtgaaaaagattagagaagcgattgagatttgcgagaaggaactgataaaactagtgttgcggaggaaggactgcgtggtacctagagcttgcaaagagttgttttggaatatgatcagaataaacaacctgttttacgcgagcattgatggctacacgtctgaaacccaaatgatgaatgaggtgaaggctgtcatgcgcattcccctcactagaccagacttaattgaaggttag

SEQ ID NO: 5 - protein, 801 aa, Delphinium grandiflorum
MYLSHPTKSPLVFPNPTTSSPRRSSSTSISAVSVDHGVKRLEKSENSLKISEESKEKISKIFTKVELSKSSYDTAWVAMVPSLDSSVSPYFPECLNWILENQHADGSWGLTQQHPLLLKDTLSSTLASILALKRWNVGEDHVNKGLHFISSNFASATDEKQRSPIGFDIIFPGMIEHAQEIGVNFHLDPTSLNSIISKRDMELHRVSTSNSEGSKLYRAYFAEGLRKSQNWEEVMKYQRKNGSLFNSPSTTAVAAAHVQDPNCLKYLHSILEEFGNAVPTSYPLDIYTQLCMIDALEKLGISRHFKNEIINVLDKTYGSWLTKDEEIFLDVSTSAMAFRILRVHGYDVSPDVLAQFDQQGFSNTLGGYLNDSGAVLEIYRASQIVLPDEVFLEEQKTWSSAYLKNELSKGSMHADRMHEWISKEVETALTYPYKPNLPRLEHRRTVEHYNVDNLRVLKSAYRPLGIDNKDLLHLAMEDFNLCQSIYQNEFKELERWVKDNRIDKLKFARQKQVYTLFSSASTLFPPELSDARLSWAKFSILTTIIDDCYDLGGSRDELINLNQVFDKWDGVTAGDFISEPVEILYYAYKNTIDDLARKAFKYQHRDITKHLVENCVEMVKSMWIEAEWMEHNVVPSLEEYNENGYVSFALGPIVLTTLYFVGPQLSEEVVRSSEYHDLFRLMSTICRNLNDLRTVQKELSEGTINGVSILMIHDPEVKTEEDSVKKIREAIEICEKELIKLVLPRKDCVVPRACKELFWNMIRINNLFYASIDGYTSETQMMNEVKAVMRIPLTRPDLIEG

SEQ ID NO: 6 - DNA, 2406 bp, Delphinium grandiflorum
  source 1..2406: mol_type = other DNA
atgtatctctcccatccaaccaagtcgcctctcgtctttccgaacccaacaacatcatcgccgaggagatcctcctccacatccatctcagctgtttctgtggatcatggtgttaagaggttggaaaaatctgaaaattctcttaagatttccgaggagagcaaggagaaaataagcaaaatcttcaccaaggttgaactttcgaaatcttcatacgacaccgcttgggttgcaatggtcccttctcttgactcctctgtatcaccctactttcccgaatgtctcaactggatcttggagaatcaacacgcggacggctcatggggccttactcagcaacaccctttattgttaaaggacacgctgtcgtcgacattggcctctatactcgcactcaaaagatggaatgtcggcgaagaccatgtgaacaagggtctccatttcattagttctaattttgcttccgccacggacgagaagcaacgtagtccaattgggtttgacatcatattccccggtatgatcgagcatgcccaggagataggagtaaacttccatttagacccaacgagtttaaattctattattagtaagagagacatggaattacatagggtatctacaagcaactcagaggggagcaaactctaccgagcctactttgcggagggactgaggaagtcgcaaaattgggaggaagtaatgaaatatcagagaaagaatggatcgttgtttaattctccttccaccactgcggttgcggccgctcacgtccaagacccgaattgcttgaagtacttgcactcgatcttggaggaattcggcaatgcagtcccgactagttatccactagacatatacacccagctctgtatgattgacgctctagagaaactgggaatctcccgacacttcaagaatgagataataaatgttttggataaaacctacggttcctggttgaccaaggacgaggaaatctttttagacgtttcgacatctgccatggcatttaggatattacgtgtacatggatatgacgtctccccagacgtactagctcaattcgaccaacaaggtttctcaaatacacttggaggatatctaaacgactcaggggctgtccttgagatatatcgggcgtcccaaattgtgctccccgatgaggtatttctggaggaacaaaaaacttggtcaagtgcttatcttaagaatgaactatccaagggttcgatgcacgccgatagaatgcatgaatggattagcaaagaggtcgaaacggcgctaacctatccctacaaacccaatttgccgcgcttagagcacaggagaaccgtggaacattacaatgtcgataacttgagagttctgaaatcagcatataggcctcttggtattgacaacaaggatttactgcatttggcgatggaagactttaatctttgtcaatcgatatatcaaaatgaattcaaggagctcgagaggtgggtgaaagacaacaggatagataagctaaagttcgcaaggcaaaagcaggtgtacacgctcttctcttccgcatcaactctatttcctccagaattaagtgacgcgcgtctctcgtgggcaaagttcagtatcctcacaactataattgacgattgctacgatttaggcggctctagagacgaactaattaacctaaaccaagtgtttgacaagtgggatggagttacagccggtgacttcatttccgagccagttgaaatactatattatgcatacaaaaatacgattgatgatcttgcaagaaaggctttcaaatatcagcatcgggatatcacaaagcatttagtggagaactgtgttgaaatggttaagtctatgtggatcgaggcagagtggatggagcacaatgtagtaccatcactggaagaatacaatgaaaatggatacgtatcgtttgctctggggcctatagttcttacaactttatattttgttgggccccaactttccgaggaagtcgtaaggagttctgagtaccatgacctatttcgactcatgagcacaatatgtcgtaacctcaatgatcttcgaacagttcagaaggaactaagcgaagggacgataaacggtgtgtccattctgatgatacacgaccctgaagtcaagacggaggaagactcggtgaaaaagattagagaagcgattgagatttgcgagaaggaactgataaaactagtgttgccgaggaaggactgcgtggtacctagagcttgcaaagagttgttttggaatatgatcagaataaacaacctgttttacgcgagcattgatggctacacgtctgaaacccaaatgatgaatgaggtgaaggctgtcatgcgcattcccctcactagaccagacttaattgaaggttag

SEQ ID NO: 7 - protein, 510 aa, Delphinium grandiflorum
MAITKEILQQLTPQTITITVVLGLFVLILLRIKKSPTNSALPSLPVVPGLPLIGNLHQLSDKKPHQTFTKWAEKYGPIYSIKTGSSTLVVLNSNDVAKEAMVTRFSSISTRKLSNALTILTLDKKIVAISDYGDFHKITKKYLISGMLGANAQKRYRGHRETMMSNMLSKLCAHIKEKPLESVNLRSIFQYELFGLALKQAYGRDLDAPFYIEGLGTKLSRYEIFEALVVDPMMGAIAVDWRDFFPYLRWIPNKGLEARIERMAFRRKAVCKALIDAQKRRRATGEILDSYVDYLLAPDLKQFSEDELIMLMWEVVIETSDTTLVTTEWAMYEIAKNRRVQELLYRELKEVCGSEKVTEDHLPRLPYLNAVFHETLRRHSPAPMIPLRYVHEDTELGGYHIPAGTQISINIFGCNMDKKQWDEPEAWKPERFLDPKFDPTDMFKSMAFGGGKRICAGAQQAMTIACMAIATYVQEFDWKLDEGQKEDVNTLGLTSYRLYPLQVHIKPRTA

SEQ ID NO: 8 - DNA, 1533 bp, Delphinium grandiflorum
  source 1..1533: mol_type = other DNA
atggccattaccaaagagatccttcaacagttaacccctcaaactattaccatcactgtagttttgggcctctttgtactcatcttgctcagaatcaagaaatctcctacaaactcagctctaccttctctacctgttgttcctgggctccctttgattgggaatttgcaccaactgagtgataagaagccacaccagactttcacaaagtgggcagagaaatatggacctatttattccattaagactggttcttctactcttgttgtcctcaactcaaatgatgtggctaaagaggctatggtgactagattctcatctatctccacaaggaagctctccaatgctttgacgatactcacactcgataaaaagattgttgccataagtgactacggggatttccacaagatcactaagaagtatctgatttcgggcatgctaggtgccaacgcgcagaagcgatatcgaggtcatagagaaaccatgatgagtaatatgttgagtaagttatgtgctcacatcaaggaaaagcctcttgaatctgtaaacttaagaagtatatttcagtatgaactctttggattagctctgaaacaagcttatggtagagatttagacgccccgttttatattgaaggtcttggtacaaaattgtcaagatatgagatatttgaggcgttagtcgtcgatccaatgatgggagcaattgctgtggactggagagactttttcccatatttgagatggattccaaacaaagggctggaagcaaggattgagcgaatggctttccggagaaaagctgtgtgtaaagcgctcatagatgcacaaaagagacgaagagctactggagagatattagacagttatgtggattacttgttagccccggacctaaagcagttctcagaggatgaactgatcatgttaatgtgggaagtggttattgagacctcagacaccactttggtcactacagaatgggctatgtatgaaatcgcaaagaacaggagagttcaggaactcctctaccgggagcttaaagaggtttgtggatctgagaaggttactgaggatcatttgccaaggctaccatacttgaacgccgtcttccatgaaactttgagaagacattctccagctccaatgatcccactaagatacgtacatgaagataccgaattgggaggctaccacatcccagctggaactcagatctccataaacatctttggatgcaacatggacaagaagcaatgggacgaaccggaagcttggaagcccgagaggttcctagaccccaaatttgatccaactgatatgttcaagtcaatggctttcgggggaggcaagagaatatgtgcaggagcgcaacaggccatgacgattgcttgcatggcgattgctacgtacgtgcaggagtttgattggaagttggatgaaggacagaaagaggatgttaatactcttggactgaccagttacagactctatcctctccaggtgcacataaaaccaagaacagcttaa

SEQ ID NO: 9 - protein, 520 aa, Delphinium grandiflorum
MAQLQPLLQWLETQQETLFRHPAALILVSIFTTLLLVRLMSGFWSKKSNMYLLPSPPTLPIIGNFHQLTTLPHRGLFKLSNKYGHLMLLHLGRAPAVIVSSAEMAREIKKTHDVAFANRPYSIASEILFYGRSNMAFAPYGEYWRQVRKICNLELLSLKRVQTFKYVREEEVAILIKTVKEASKTKLPMNLTENLLGLTNNIVSRCALGKKSRGEGSNMKLGVLSRQFIQMLEAFSFKDHFPILGFLDHVTGLYRKMKYVSGELDAFLEETIDEHEAQKTQDYHEDREDFVDLLLRVKRDNTLDMDFTRKHIKALVLDMYLGGTDTSSTTIEWTMTELLRHPFAMKKAQEEIRRVVGNKPQVEEDDVNHMDYLKCALKETLRLHAPVPLIYLESSVNTDIKGVKVPAKTKVIVNIWAIQRDGKSWDNPEEFIPERFMNNPVDFRGQDYEYIPFGSGRRGCPGMTFGLSMVEYILANILYCFDWNLPAGMTIADIDMDESFGSTVSKKDPLMLIPTLKPTN

SEQ ID NO: 10 - DNA, 1563 bp, Delphinium grandiflorum
  source 1..1563: mol_type = other DNA
atggctcagttgcaaccattgctgcaatggctagaaacccagcaagaaaccctgtttcgccatcccgcggctctcattcttgtctccatcttcaccactctccttctagtgaggcttatgagtggcttttggtctaaaaagtccaatatgtacctccttccatcacctccaactctcccgatcatcggaaatttccaccaactcaccacacttcctcaccgtggtctgtttaaactctccaacaagtacggtcacctgatgcttcttcatttggggcgtgcgcccgccgtgatagtctcctcggccgagatggccagagagatcaagaaaacccacgacgtggcgtttgccaacaggccttactccatagccagtgagattctcttctacgggcgcagcaacatggcgtttgccccgtacggggaatactggaggcaggtcagaaagatatgtaacttggaactcttgagtttgaagagagttcagacttttaagtacgtaagggaggaagaggtggcgattctgatcaagactgtaaaagaggcttcgaagacaaaactcccgatgaacctaaccgagaatctactcggactcaccaacaacatagtgtcgaggtgcgctcttgggaagaaaagccggggagaaggcagtaacatgaaattaggggtgttgtcaagacagttcatccagatgttggaagctttcagcttcaaagaccattttccaatcttggggtttttggatcacgtgaccgggttgtaccgaaagatgaaatatgtttctggagagctggacgcttttctcgaggaaactatcgacgaacacgaagcgcagaagacgcaagattatcacgaggatagagaagactttgttgatctcctactgagggtgaaaagagacaacaccctagacatggatttcactaggaaacacatcaaagctctagttctggacatgtatcttgggggaacagacacttcatcaaccaccatagaatggactatgacggagctgctgaggcatccgtttgcgatgaaaaaagcccaagaagagatcagaagagtggttgggaacaagccccaggtggaagaggacgacgtcaatcatatggactacctaaaatgcgccctcaaagaaacccttcgcctacatgcacccgtgcccttgatctacctcgagtcctcggtcaataccgatataaagggagttaaagtcccagccaaaacaaaagtgatagtgaacatatgggcaattcaaagggacggaaaatcgtgggacaatccggaagaattcatcccagaaaggtttatgaacaatccggttgatttcagagggcaggattatgagtacatcccgttcgggtcgggacgaagaggctgcccgggtatgacattcggtctgtctatggtagagtatattttggcaaatatactctactgtttcgactggaatctgcctgctgggatgaccatagccgatatcgacatggatgaaagtttcggtagcactgtcagtaaaaaagatcctctcatgctcattccaaccctcaaacctaccaattag

SEQ ID NO: 11 - protein, 476 aa, Delphinium grandiflorum
MELTQAQAWWSALVFTILPFLVWLVFSWNELRYVKTQSSDGGKLPPGHLGLPVIGQLLSFIWYFRIRRNPDDFVHSMRKRYGDADGIYRSYLFGSPAIIGCSPDFNKFVLQSSNLFQATRRQKDIFGHNSVAVVNGKAHYRLRGYINNTISTPDALKKITICIQPNIVSSLQSWAEKGKIKGVYDIKKVFFETICIIITSFKPGPAIDMLDQHFHAILDGLGEKGTKFHLAVQSKKTLTEVFKKEIDKRTQHGIPSEDQNDLMERLMRMRDEDGEPLSDDEVIDNIVTCIMGGYESPFQLAIWALYFLAKNNDVLQKLREENLAIDKKGELLTSEDLAHLKYTKKVVEETLRMANIGTFFVRTAEKDVTYRGNKIPKNWLILLWTRYLHNNTENFEDPMKFNPDRWDETPKPGTFQPFGLGPRICPANMLSKTQLVIFIHHVVVGYKWELTNPNVKISYVPQPMPSDGLEINFSKL

SEQ ID NO: 12 - DNA, 1431 bp, Delphinium grandiflorum
  source 1..1431: mol_type = other DNA
atggagctcacacaagcacaggcatggtggtctgctcttgtctttactatcttaccttttcttgtgtggctcgtcttctcatggaatgagctcagatatgtgaaaactcagtccagtgatggaggcaagcttccaccagggcatcttggtttgccagttatcggccaactcctcagcttcatttggtatttcagaattcgccggaaccccgatgatttcgtccattcaatgagaaaaagatacggagatgctgatggaatatatcgaagctacctctttggatctccggcaatcatcggctgctccccagatttcaacaagtttgtcctacaatcaagcaatttgtttcaagctacccgacgtcaaaaggatatttttggccataattctgttgcagtagttaatggtaaagcacattacagacttaggggttacatcaacaatacaatcagtactcctgatgctctaaagaagatcacaatttgtatacaacccaatatagtctcctccctccagtcatgggcagagaaaggtaaaatcaaaggggtatatgacatcaagaaggtattctttgaaaccatctgtattataatcactagcttcaaacctggccccgcaatagatatgcttgatcaacactttcatgccattcttgacggacttggagaaaaagggacaaagtttcacctagcagttcagagtaaaaagacattgactgaagttttcaagaaagaaattgataaaagaacgcaacatggtattccatcagaggaccaaaatgatctgatggaaagattgatgagaatgagagatgaggatggagaaccattaagtgatgatgaggtgattgataatattgtgacttgtatcatgggtggctatgaatcacctttccaacttgcgatatgggctctttactttctagccaagaacaatgatgtgcttcaaaaactccgggaagaaaatctagccatagataagaaaggagaattgttaacaagtgaagatcttgcacacttgaagtacacgaagaaggtggtggaagaaactctaagaatggcaaacattggaactttctttgttaggacagcagaaaaggatgttacttatcgaggtaataaaataccaaagaattggcttatacttctatggacgcgctatcttcataataatacagaaaattttgaagaccccatgaagttcaatcctgatagatgggatgaaactccaaagcccggcacatttcaaccatttggtttgggtccaaggatttgtccagcaaacatgctttctaaaactcaacttgttatttttattcatcatgtggtggtcggatacaagtgggaactgacaaatccaaatgtgaaaataagctatgttccacaaccaatgccatcagatggattggagattaatttcagtaaattatag

SEQ ID NO: 13 - protein, 517 aa, Delphinium grandiflorum
MENVVQQVATSNNPFFLLFLSLVFLLLVLKFKFTTNTTNPKFPPSPRKLPFIGNAHQLVGGALHHVLHSLSQKHGPLMFLHLVSRPTLVVSDANTAREVMKTYDHIFSSRPQLGIPNRLLYGKDVAFAPYGEYWRQVKKICVTQLLSAKKVQSFRVVREEEVALAMDQMDQIEAASSGINLSELFAGILGSVVCRVALGRKYDTQGGGGRKFKKIVTEMTNLLGVTNIADLVPSLGWLNHFNGLNARVEKNFRDIDSFLDGVIEEHLAKKRGGEVEEEDIVDIMLRNEEDSTLGIPITREATKGVVLDMFAAGIETSSIVLQWAMSELMKHPEIMLEVQKEVRDVAKGKHILTENDINEMHQLKSVIKETMRLHPPFPLLILRESVKDVNIEGYHVPAKTTVIINAVAIGKDQMWWEEPERFLPKRFMNGRSTMVDFKGQDFQLIPFGAGRRICPGMLFATSITELTFANLLNRFDWIMPNGVASDELDMKEGSGITIHRKFDLVLIAKPYHEICVE

SEQ ID NO: 14 - DNA, 1554 bp, Delphinium grandiflorum
  source 1..1554: mol_type = other DNA
atggagaatgtagtacagcaagtagctacttcaaataatcccttcttcctcctcttcctctctcttgtctttcttcttctagtgctcaagtttaagtttactacaaacacaactaaccccaaattccctccttccccacggaagcttcccttcataggaaacgcacaccaactcgtcgggggtgctcttcaccatgttctccactcgctatcccaaaagcatggccccttgatgttcttgcaccttgtttccagaccaaccctagttgtatcggatgctaataccgcccgagaagttatgaagacttacgatcatatcttttcaagtaggcctcaacttgggattcctaaccgactgctatacggtaaggatgttgcctttgcaccctacggggagtactggaggcaagtgaagaagatatgcgtcacacagcttttaagtgctaagaaggtccagtcgtttcgggttgttagagaagaagaagtagctcttgccatggatcaaatggatcaaatagaggctgcctcttcggggattaatttgagcgaattatttgctggtattttgggtagtgtagtttgtagggttgccttggggagaaagtatgatacacaaggaggaggtggtaggaagtttaagaagattgtaactgaaatgacaaatttgttgggagttacaaatatagccgacctagtaccctcacttggttggttaaatcattttaatgggttgaatgcgcgggttgagaagaatttccgcgacattgattctttcttagatggagtaattgaagaacatttggccaagaagagaggtggtgaagtagaagaagaagatatagtagacattatgctcaggaatgaagaagactctactcttggaattcccataacaagagaagccactaaaggagtcgtactggatatgtttgcagctgggatcgaaacttcgtcaatagttttacagtgggcaatgtccgagctgatgaaacatcctgaaatcatgttagaagtacaaaaggaggtcagagatgttgctaaaggaaagcacatattaactgaaaatgatataaacgaaatgcaccaattgaaatcagttattaaagagactatgagattgcatcctccatttcctttgttgattcttcgtgaatcggtaaaagatgtaaacattgagggctatcacgttcctgcaaaaacaactgtcataatcaatgcagttgcaatcggtaaagatcaaatgtggtgggaagagcctgagagatttttgccaaagagatttatgaacggtaggagtacaatggttgattttaaaggacaagattttcaactaattccatttggagcgggtaggagaatatgccctggaatgctttttgcaacatccataactgaacttacttttgcgaatcttcttaacagatttgattggatcatgccaaatggagtggccagtgatgaattagatatgaaagaaggttctgggattacaattcataggaaatttgatctcgttcttattgcaaagccatatcatgaaatatgtgttgaataa